SEQUENCE LISTING (81 sequences)

SEQ ID NO: 1 - protein, 122 aa, synthetic construct
  REGION 1..122: note = nanobody
QVQLQESGGGLVQAGGSLRLSCAASGTIFATYYMGWYRQAPGKERELVAAIAYGGTTYYADSVKGRFTISRDNAKNTVYLQMNSLKPEDTAVYYCAAEQYEQYRTLPPYDYWGQGTQVTVSS

SEQ ID NO: 2 - protein, 118 aa, synthetic construct
  REGION 1..118: note = nanobody
QVQLQESGGGLVQAGGSLRLSCAASGYIFGIVYMGWYRQAPGKERELVATIDTGTNTYYADSVKGRFTISRDNAKNTVYLQMNSLKPEDTAVYYCAAEGRDYRDYDYWGQGTQVTVSS

SEQ ID NO: 3 - protein, 118 aa, synthetic construct
  REGION 1..118: note = nanobody
QVQLQESGGGLVQAGGSLRLSCAASGTISDTRYMGWYRQAPGKERELVAAIDYGSTTYYADSVKGRFTISRDNAKNTVYLQMNSLKPEDTAVYYCAAEYVLSKDHEYWGQGTQVTVSS

SEQ ID NO: 4 - protein, 118 aa, synthetic construct
  REGION 1..118: note = nanobody
QVQLQESGGGLVQAGGSLRLSCAASGSIFERAYMGWYRQAPGKEREFVAAIGYGTNTNYADSVKGRFTISRDNAKNTVYLQMNSLKPEDTAVYYCAALARDVYSYNYWGQGTQVTVSS

SEQ ID NO: 5 - protein, 122 aa, synthetic construct
  REGION 1..122: note = nanobody
QVQLQESGGGLVQAGGSLRLSCAASGTIFSFSYMGWYRQAPGKERELVAAIARGTTTYYADSVKGRFTISRDNAKNTVYLQMNSLKPEDTAVYYCAAEHNWGEPYRSYYDYWGQGTQVTVSS

SEQ ID NO: 6 - protein, 122 aa, synthetic construct
  REGION 1..122: note = nanobody
QVQLQESGGGLVQAGGSLRLSCAASGYISDYLRMGWYRQAPGKERELVATIARGGITNYADSVKGRFTISRDNAKNTVYLQMNSLKPEDTAVYYCAARLPYYKYNGFVLVYWGQGTQVTVSS

SEQ ID NO: 7 - protein, 9 aa, synthetic construct
  REGION 1..9: note = nanobody
GTIFATYYM

SEQ ID NO: 8 - protein, 14 aa, synthetic construct
  REGION 1..14: note = nanobody
ELVAAIAYGGTTYY

SEQ ID NO: 9 - protein, 16 aa, synthetic construct
  REGION 1..16: note = nanobody
AAEQYEQYRTLPPYDY

SEQ ID NO: 10 - protein, 9 aa, synthetic construct
  REGION 1..9: note = nanobody
GYIFGIVYM

SEQ ID NO: 11 - protein, 14 aa, synthetic construct
  REGION 1..14: note = nanobody
ELVATIDTGTNTYY

SEQ ID NO: 12 - protein, 12 aa, synthetic construct
  REGION 1..12: note = nanobody
AAEGRDYRDYDY

SEQ ID NO: 13 - protein, 9 aa, synthetic construct
  REGION 1..9: note = nanobody
GTISDTRYM

SEQ ID NO: 14 - protein, 14 aa, synthetic construct
  REGION 1..14: note = nanobody
ELVAAIDYGSTTYY

SEQ ID NO: 15 - protein, 12 aa, synthetic construct
  REGION 1..12: note = nanobody
AAEYVLSKDHEY

SEQ ID NO: 16 - protein, 9 aa, synthetic construct
  REGION 1..9: note = nanobody
GSIFERAYM

SEQ ID NO: 17 - protein, 14 aa, synthetic construct
  REGION 1..14: note = nanobody
EFVAAIGYGTNTNY

SEQ ID NO: 18 - protein, 12 aa, synthetic construct
  REGION 1..12: note = nanobody
AALARDVYSYNY

SEQ ID NO: 19 - protein, 9 aa, synthetic construct
  REGION 1..9: note = nanobody
GTIFSFSYM

SEQ ID NO: 20 - protein, 14 aa, synthetic construct
  REGION 1..14: note = nanobody
ELVAAIARGTTTYY

SEQ ID NO: 21 - protein, 16 aa, synthetic construct
  REGION 1..16: note = nanobody
AAEHNWGEPYRSYYDY

SEQ ID NO: 22 - protein, 9 aa, synthetic construct
  REGION 1..9: note = nanobody
GYISDYLRM

SEQ ID NO: 23 - protein, 14 aa, synthetic construct
  REGION 1..14: note = nanobody
ELVATIARGGITNY

SEQ ID NO: 24 - protein, 16 aa, synthetic construct
  REGION 1..16: note = nanobody
AARLPYYKYNGFVLVY

SEQ ID NO: 25 - protein, 118 aa, synthetic construct
  REGION 1..118: note = nanobody
QVQLQESGGGLVQAGGSLRLSCAASGTISGSGSMGWYRQAPGKEREFVAAINVGSNTYYADSVKGRFTISRDNAKNTVYLQMNSLKPEDTAVYYCAVRFGYYYRHTYWGQGTQVTVSS

SEQ ID NO: 26 - protein, 118 aa, synthetic construct
  REGION 1..118: note = nanobody
QVQLQESGGGLVQAGGSLRLSCAASGSIFSRFYMGWYRQAPGKEREFVAGISAGGTTYYADSVKGRFTISRDNAKNTVYLQMNSLKPEDTAVYYCAVVAGRLLRYRYWGQGTQVTVSS

SEQ ID NO: 27 - protein, 122 aa, synthetic construct
  REGION 1..122: note = nanobody
QVQLQESGGGLVQAGGSLRLSCAASGTISYHGTMGWYRQAPGKEREFVAAIARGGNTNYADSVKGRFTISRDNAKNTVYLQMNSLKPEDTAVYYCAALLRRSGYITSSFLYWGQGTQVTVSS

SEQ ID NO: 28 - protein, 117 aa, synthetic construct
  REGION 1..117: note = nanobody
QVQLQESGGGLVQAGGSLRLSCAASGTISRYTTMGWYRQAPGKERELVAGITPGGSTYYADSVKGRFTISRDNAKNTVYLQMNSLKPEDTAVYYCAARDYWAKLSYWGQGTQVTVSS

SEQ ID NO: 29 - protein, 122 aa, synthetic construct
  REGION 1..122: note = nanobody
QVQLQESGGGLVQAGGSLRLSCAASGSIFSRTSMGWYRQAPGKERELVAGITWGGNTYYADSVKGRFTISRDNAKNTVYLQMNSLKPEDTAVYYCAVLVPIGRDVKGYHRYWGQGTQVTVSS

SEQ ID NO: 30 - protein, 122 aa, synthetic construct
  REGION 1..122: note = nanobody
QVQLQESGGGLVQAGGSLRLSCAASGTIFRYAVMGWYRQAPGKEREFVAAINSGTNTNYADSVKGRFTISRDNAKNTVYLQMNSLKPEDTAVYYCAALYRNPAFPIYAHTYWGQGTQVTVSS

SEQ ID NO: 31 - protein, 122 aa, synthetic construct
  REGION 1..122: note = nanobody
QVQLQESGGGLVQAGGSLRLSCAASGTIFSYGYMGWYRQAPGKEREFVAGISRGATTNYADSVKGRFTISRDNAKNTVYLQMNSLKPEDTAVYYCAVVGLRVQYQAYLYRYWGQGTQVTVSS

SEQ ID NO: 32 - protein, 118 aa, synthetic construct
  REGION 1..118: note = nanobody
QVQLQESGGGLVQAGGSLRLSCAASGSISRFGVMGWYRQAPGKEREFVAAIASGTTTYYADSVKGRFTISRDNAKNTVYLQMNSLKPEDTAVYYCAAREYGYGGHLYWGQGTQVTVSS

SEQ ID NO: 33 - protein, 121 aa, synthetic construct
  REGION 1..121: note = nanobody
QVQLQESGGGLVQAGGSLRLSCAASGSIFYYSRMGWYRQAPGKERELVAGIGRGTTYYADSVKGRFTISRDNAKNTVYLQMNSLKPEDTAVYYCAVYPNYQWAYAVLHGYWGQGTQVTVSS

SEQ ID NO: 34 - protein, 122 aa, synthetic construct
  REGION 1..122: note = nanobody
QVQLQESGGGLVQAGGSLRLSCAASGSISYYLYMGWYRQAPGKEREFVAAINRGATTYYADSVKGRFTISRDNAKNTVYLQMNSLKPEDTAVYYCAVRAIQTSSERRYFTYWGQGTQVTVSS

SEQ ID NO: 35 - protein, 122 aa, synthetic construct
  REGION 1..122: note = nanobody
QVQLQESGGGLVQAGGSLRLSCAASGTISLARYMGWYRQAPGKEREFVAGITYGTTTYYADSVKGRFTISRDNAKNTVYLQMNSLKPEDTAVYYCAAYLRSTTSGYLYHRYWGQGTQVTVSS

SEQ ID NO: 36 - protein, 118 aa, synthetic construct
  REGION 1..118: note = nanobody
QVQLQESGGGLVQAGGSLRLSCAASGTVSYAMGWYRQAPGKEREFVAAITLGSNTNYADSVKGRFTISRDNAKNTVYLQMNSLKPEDTAVYYCAAYRRYGKTLYLLYWGQGTQVTVSS

SEQ ID NO: 37 - protein, 122 aa, synthetic construct
  REGION 1..122: note = nanobody
QVQLQESGGGLVQAGGSLRLSCAASGTISSDAWMGWYRQAPGKERELVASISTGATTYYADSVKGRFTISRDNAKNTVYLQMNSLKPEDTAVYYCAAVPRRRGYYTYYFRYWGQGTQVTVSS

SEQ ID NO: 38 - protein, 122 aa, synthetic construct
  REGION 1..122: note = nanobody
QVQLQESGGGLVQAGGSLRLSCAASGYIFQYASMGWYRQAPGKERELVAGISAGATTYYADSVKGRFTISRDNAKNTVYLQMNSLKPEDTAVYYCAARWYDLSQYPRRHHYWGQGTQVTVSS

SEQ ID NO: 39 - protein, 9 aa, synthetic construct
  REGION 1..9: note = nanobody
GTISGSGSM

SEQ ID NO: 40 - protein, 14 aa, synthetic construct
  REGION 1..14: note = nanobody
EFVAAINVGSNTYY

SEQ ID NO: 41 - protein, 12 aa, synthetic construct
  REGION 1..12: note = nanobody
AVRFGYYYRHTY

SEQ ID NO: 42 - protein, 9 aa, synthetic construct
  REGION 1..9: note = nanobody
GSIFSRFYM

SEQ ID NO: 43 - protein, 14 aa, synthetic construct
  REGION 1..14: note = nanobody
EFVAGISAGGTTYY

SEQ ID NO: 44 - protein, 12 aa, synthetic construct
  REGION 1..12: note = nanobody
AVVAGRLLRYRY

SEQ ID NO: 45 - protein, 9 aa, synthetic construct
  REGION 1..9: note = nanobody
GTISYHGTM

SEQ ID NO: 46 - protein, 14 aa, synthetic construct
  REGION 1..14: note = nanobody
EFVAAIARGGNTNY

SEQ ID NO: 47 - protein, 16 aa, synthetic construct
  REGION 1..16: note = nanobody
AALLRRSGYITSSFLY

SEQ ID NO: 48 - protein, 9 aa, synthetic construct
  REGION 1..9: note = nanobody
GTISRYTTM

SEQ ID NO: 49 - protein, 14 aa, synthetic construct
  REGION 1..14: note = nanobody
ELVAGITPGGSTYY

SEQ ID NO: 50 - protein, 11 aa, synthetic construct
  REGION 1..11: note = nanobody
AARDYWAKLSY

SEQ ID NO: 51 - protein, 9 aa, synthetic construct
  REGION 1..9: note = nanobody
GSIFSRTSM

SEQ ID NO: 52 - protein, 14 aa, synthetic construct
  REGION 1..14: note = nanobody
ELVAGITWGGNTYY

SEQ ID NO: 53 - protein, 16 aa, synthetic construct
  REGION 1..16: note = nanobody
AVLVPIGRDVKGYHRY

SEQ ID NO: 54 - protein, 9 aa, synthetic construct
  REGION 1..9: note = nanobody
GTIFRYAVM

SEQ ID NO: 55 - protein, 14 aa, synthetic construct
  REGION 1..14: note = nanobody
EFVAAINSGTNTNY

SEQ ID NO: 56 - protein, 16 aa, synthetic construct
  REGION 1..16: note = nanobody
AALYRNPAFPIYAHTY

SEQ ID NO: 57 - protein, 10 aa, synthetic construct
  REGION 1..10: note = nanobody
GTIFSYGYMG

SEQ ID NO: 58 - protein, 14 aa, synthetic construct
  REGION 1..14: note = nanobody
EFVAGISRGATTNY

SEQ ID NO: 59 - protein, 16 aa, synthetic construct
  REGION 1..16: note = nanobody
AVVGLRVQYQAYLYRY

SEQ ID NO: 60 - protein, 9 aa, synthetic construct
  REGION 1..9: note = nanobody
GSISRFGVM

SEQ ID NO: 61 - protein, 14 aa, synthetic construct
  REGION 1..14: note = nanobody
EFVAAIASGTTTYY

SEQ ID NO: 62 - protein, 12 aa, synthetic construct
  REGION 1..12: note = nanobody
AAREYGYGGHLY

SEQ ID NO: 63 - protein, 9 aa, synthetic construct
  REGION 1..9: note = nanobody
GSIFYYSRM

SEQ ID NO: 64 - protein, 13 aa, synthetic construct
  REGION 1..13: note = nanobody
ELVAGIGRGTTYY

SEQ ID NO: 65 - protein, 16 aa, synthetic construct
  REGION 1..16: note = nanobody
AVYPNYQWAYAVLHGY

SEQ ID NO: 66 - protein, 9 aa, synthetic construct
  REGION 1..9: note = nanobody
GSISYYLYM

SEQ ID NO: 67 - protein, 14 aa, synthetic construct
  REGION 1..14: note = nanobody
EFVAAINRGATTYY

SEQ ID NO: 68 - protein, 16 aa, synthetic construct
  REGION 1..16: note = nanobody
AVRAIQTSSERRYFTY

SEQ ID NO: 69 - protein, 9 aa, synthetic construct
  REGION 1..9: note = nanobody
GTISLARYM

SEQ ID NO: 70 - protein, 14 aa, synthetic construct
  REGION 1..14: note = nanobody
EFVAGITYGTTTYY

SEQ ID NO: 71 - protein, 16 aa, synthetic construct
  REGION 1..16: note = nanobody
AAYLRSTTSGYLYHRY

SEQ ID NO: 72 - protein, 7 aa, synthetic construct
  REGION 1..7: note = nanobody
GTVSYAM

SEQ ID NO: 73 - protein, 14 aa, synthetic construct
  REGION 1..14: note = nanobody
EFVAAITLGSNTNY

SEQ ID NO: 74 - protein, 14 aa, synthetic construct
  REGION 1..14: note = nanobody
AAYRRYGKTLYLLY

SEQ ID NO: 75 - protein, 9 aa, synthetic construct
  REGION 1..9: note = nanobody
GTISSDAWM

SEQ ID NO: 76 - protein, 14 aa, synthetic construct
  REGION 1..14: note = nanobody
ELVASISTGATTYY

SEQ ID NO: 77 - protein, 16 aa, synthetic construct
  REGION 1..16: note = nanobody
AAVPRRRGYYTYYFRY

SEQ ID NO: 78 - protein, 9 aa, synthetic construct
  REGION 1..9: note = nanobody
GYIFQYASM

SEQ ID NO: 79 - protein, 14 aa, synthetic construct
  REGION 1..14: note = nanobody
ELVAGISAGATTYY

SEQ ID NO: 80 - protein, 16 aa, synthetic construct
  REGION 1..16: note = nanobody
AARWYDLSQYPRRHHY

SEQ ID NO: 81 - DNA, 39 bp, synthetic construct
  misc_feature 1..39: note = bungarotoxin-binding site
  source 1..39: mol_type = other DNA
tggcggtactacgagagcagcctggagccctaccccgac

(19) United States
(12) Patent Application Publication
(10) Pub. No.: US 2023/0235084 A1
(43) Pub. Date: Jul. 27, 2023

(54) COMPOSITIONS AND METHODS FOR TARGETED PROTEIN STABILIZATION BY REDIRECTING ENDOGENOUS DEUBIQUITINASES

(71) Applicant: THE TRUSTEES OF COLUMBIA UNIVERSITY IN THE CITY OF NEW YORK, New York, NY (US)

(72) Inventors: Scott KANNER, New York, NY (US); Henry M. COLECRAFT, New York, NY (US)

(21) Appl. No.: 17/864,389

(22) Filed: Jul. 13, 2022

Related U.S. Application Data
(63) Continuation of application No. PCT/US2021/013390, filed on Jan. 14, 2021.
(60) Provisional application No. 62/961,082, filed on Jan. 14, 2020.

(51) Int. Cl. C07K 16/40; C07K 16/28; A61P 11/00

(52) U.S. Cl. CPC C07K 16/40; A61P 11/00; C07K 16/28; C07K 2317/35; C07K 2317/569; C07K 2317/622; C07K 2319/33

(57) ABSTRACT

The present disclosure provides, inter alia, bivalent nanobody molecules and methods for treating or ameliorating the effects of a disease, such as long QT syndrome, or cystic fibrosis, in a subject, using the bivalent nanobody molecules disclosed herein. Also provided are methods of identifying and preparing nanobody binders that target proteins of interest.

### CROSS REFERENCE TO RELATED APPLICATIONS

The present application is a continuation application of PCT International Application No. PCT/US2021/013390, filed Jan. 14, 2021, which claims benefit of U.S. Provisional Pat. Application Serial No. 62/961,082, filed on Jan. 14, 2020, which applications are incorporated by reference herein in their entireties.

### GOVERNMENT FUNDING

This invention was made with government support under grant no. HL122421, awarded by the National Institutes of Health. The government has certain rights in the invention.

### FIELD OF THE DISCLOSURE

The present disclosure provides, inter alia, bivalent nanobody molecules and methods for treating or ameliorating the effects of a disease, such as long QT syndrome, or cystic fibrosis, in a subject, using such bivalent molecules.

### INCORPORATION BY REFERENCE OF SEQUENCE LISTING

This application contains references to amino acids and/or nucleic acid sequences that have been filed concurrently herewith as sequence listing XML file “CU19201-000768-seq.xml”, file size of 103,469 bytes, created on Feb. 1, 2023. The aforementioned sequence listing is hereby incorporated by reference in its entirety pursuant to 37 C.F.R. § 1.52(e)(5).

### BACKGROUND OF THE DISCLOSURE

Protein stability is critical for the proper function of all proteins in the cell. Many disease processes stem from deficits in the stability or expression of one or more proteins, ranging from inherited mutations that destabilize ion channels (i.e. cystic fibrosis, CFTR), to viral-mediated elimination of host defenses (i.e. MHCI receptors) and degradation of cell cycle inhibitors in tumor cell proliferation (i.e. p27, p21). Ubiquitin is a key post-translational modification that is a master regulator of protein turnover and degradation. Nevertheless, the widespread biological role and promiscuity of ubiquitin signaling has provided a significant barrier in developing therapeutics that target this pathway to selectively stabilize a given protein-of-interest.

Ubiquitination is mediated by a step-wise cascade of three enzymes (E1, E2, E3), resulting in the covalent attachment of the 76-residue ubiquitin to exposed lysines of a target protein. Ubiquitin itself contains seven lysines (K6, K11, K27, K29, K33, K48, K63) that, together with its N-terminus (Met1), can serve as secondary attachment points, resulting in a diversity of polymeric chains, differentially interpreted as sorting, trafficking, or degradative signals. Ubiquitination has been associated with inherited disorders (cystic fibrosis, cardiac arrhythmias, epilepsy, and neuropathic pain), metabolic regulation (cholesterol homeostasis), infectious disease (hijacking of host system by viral and bacterial pathogens), and cancer biology (degradation of tumor suppressors, evasion of immune surveillance).

Deubiquitinases (DUBs) are specialized isopeptidases that provide salience to ubiquitin signaling through the revision and removal of ubiquitin chains. There are over 100 human DUBs, comprising 6 distinct families: 1) the ubiquitin specific proteases (USP) family, 2) the ovarian tumor proteases (OTU) family, 3) the ubiquitin C-terminal hydrolases (UCH) family, 4) the Josephin domain family (Josephin), 5) the motif interacting with ubiquitin-containing novel DUB family (MINDY), and 6) the JAB1/MPN/Mov34 metalloenzyme domain family (JAMM). Each class of DUBs have their own distinct catalytic properties, with the USP family hydrolyzing all ubiquitin chain types, in stark contrast to the JAMM and OTU families, which contains a diverse set of enzymes with distinct ubiquitin linkage preferences. Recently, DUBs have garnered interest as drug targets, with multiple companies pursuing DUB inhibitors. However, targeting DUBs for therapy has challenges, owing to promiscuity in DUB regulation pathways wherein individual DUBs typically target multiple protein substrates, and particular substrates can be regulated by multiple DUB types.

Ion channelopathies characterized by abnormal trafficking, stability, and dysfunction of ion channels/receptors constitute a significant unmet clinical need in human disease. Inherited ion channelopathies are rare diseases that encompass a broad range of disorders in the nervous system (epilepsy, migraine, neuropathic pain), cardiovascular system (long QT syndrome, Brugada syndrome), respiratory (cystic fibrosis), endocrine (diabetes, hyperinsulinemic hypoglycemia), and urinary (Bartter syndrome, diabetes insipidus) system. Although next generation genomic sequencing has revealed a rapidly expanding list of thousands of channel mutations (with diverse underlying mechanisms of pathology), these rare diseases are almost exclusively treated symptomatically. For example, cystic fibrosis, the most common lethal genetic disease in Caucasians arises due to defects in the cystic fibrosis transmembrane conductance regulator (CFTR), a chloride ion channel. The most studied mutation (ΔF508), accounts for ~85% of all cases, and causes channel misfolding and ubiquitin-dependent trafficking defects. In another devastating disease, Long QT Syndrome, over 500 mutations in two channels (KCNQ1, hERG) encompasses nearly 90% of all inherited cases. Trafficking deficits in the two channels is the mechanistic basis for a majority of the disease-causing mutations. As such, understanding the underlying cause of loss-of-function is critical for employing a personalized strategy to treat the underlying functional deficit in each disease.

### SUMMARY OF THE DISCLOSURE

The present disclosure provides a bivalent molecule comprising: a) a deubiquitinase (DUB) binder; b) a target binder; and c) a variable linker between the DUB binder and the target binder, wherein the DUB binder is selected from intracellular antibody fragments, scFvs, nanobodies, antibody mimetics, monobodies, DARPins, lipocalins, and targeting sequences.

The present disclosure also provides a method of treating or ameliorating the effects of a disease in a subject, comprising administering to the subject an effective amount of a bivalent molecule disclosed herein.

The present disclosure further provides a method of identifying and preparing a nanobody binder targeting a protein of interest, comprising: a) constructing a naive yeast library that expresses synthetic nanobodies; b) incubating the naive yeast library with the protein of interest; c) selecting yeast cells expressing nanobodies that bind to the protein of interest by magnetic-activated cell sorting (MACS); d) amplifying the selected cells and constructing an enriched yeast library; e) incubating the enriched yeast library with the protein of interest; f) selecting yeast cells expressing nanobodies that bind to the protein of interest by fluorescence activated cell sorting (FACS); g) amplifying the selected cells and constructing a further enriched yeast library; h) repeating steps e) to g) twice; and i) sorting the selected yeast cells as single cells and cultivating as monoclonal colonies for binding validation and plasmid isolation.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1H show that enDUBs reverse NEDD4L-mediated ubiquitination of KCNQ1. FIG. 1A is schematic of targeted deubiquitination via enDUBs (nano, PDB: 3K1K). Inset, Modular domains of OTUD1 and enDUB-O1. In FIG. 1B, Left, KCNQ1 pulldowns probed with anti-KCNQ1 antibody from HEK293 cells expressing KCNQ1-YFP ± NEDD4L with nano alone or enDUB-O1. Right, Anti-ubiquitin labeling of KCNQ1 pulldowns after stripping previous blot. FIG. 1C shows relative KCNQ1 ubiquitination computed by ratio of anti-ubiquitin to anti-KCNQ1 signal intensity (n = 4; mean). **p<0.002, one-way ANOVA with Tukey’s multiple comparison test. FIG. 1D provides flow cytometry dot plots showing surface (BTX₆₄₇ fluorescence) and total (YFP fluorescence) KCNQ1 expression in cells expressing BBS-KCNQ1- YFP. Vertical and horizontal lines represent thresholds for YFP and BTX₆₄₇-positive cells, respectively, based on analyses of single color controls. FIGS. 1E and 1F show quantification of flow cytometry experiments for surface (FIG. 1E) and total KCNQ1 expression (FIG. 1F), analyzed from YFP- and CFP-positive cells (n ≥ 5000 cells per experiment; N = 4; mean ± s.e.m). Data are normalized to values from the control group, KCNQ1 without NEDD4L (dotted line). *p<0.01, unpaired two-tailed Student’s t test. FIG. 1G shows exemplar family of KCNQ1 currents from whole-cell patch clamp measurements in CHO cells. FIG. 1H provides population I-V curves for nano (black circle, n = 9), nano + NEDD4L (red square, n = 9), and enDUB-O1 + NEDD4L (blue triangle, n = 12). *p<0.01 versus nano + NEDD4L, two-way ANOVA with Tukey’s multiple comparison test.

FIGS. 2A-2I show that enDUBs rescue trafficking-deficient mutant LQT1 channels. In FIG. 2A, Left, Schematic of LQT1 patient mutations along C-terminus of KCNQ1. Right, Quantification of flow cytometry experiments for surface expression (BTX₆₄₇) of LQT1 mutant channels in presence of nano alone (red) or enDUB-O1 (blue), analyzed from YFP- and CFP-positive cells (n ≥ 5000 cells per experiment; N = 3; mean ± s.e.m). Data are normalized to values from the WT KCNQ1 control group (dotted line). *p<0.05, unpaired two-tailed Student’s t test. Right inset, Confocal image of live cells expressing BBS-tagged WT KCNQ1-YFP (top) or G589D-YFP + nano (middle) or enDUB-O1 (bottom), stained with BTX₆₄₇ (magenta). FIG. 2B shows exemplar families of WT and mutant KCNQ1 currents reconsitituted in CHO cells. FIG. 2C shows population I-V curves for WT + nano (black square, n = 10), R591H + nano (pink triangle, n = 8), and R591H + nano + ML277 (red triangle, n = 13). **p<0.001, two-way ANOVA with Tukey’s multiple comparison test. FIG. 2D shows population I-V curves for R591H + enDUB-O1 (light blue circle, n = 9), and R591H + enDUB-O1 + ML277 (blue circle, n = 9). Data for WT KCNQ1 and R591H + nano are reproduced from FIG. 2C (black and pink lines). *p<0.01, **p<0.001, two-way ANOVA with Tukey’s multiple comparison test. FIG. 2E shows confocal image of adult guinea pig cardiomyocytes expressing WT KCNQ1-YFP (top) or G589D-YFP + nano (middle) or enDUB-O1 (bottom). FIG. 2F shows average current response of slow voltage ramp to +100 mV from cardiomyocytes expressing WT KCNQ1-YFP (left; n = 17) or G589D-YFP (right) + nano alone (red; n = 16) or enDUB-O1 (blue; n = 14) (mean ± s.e.m). FIG. 2G shows quantification of Iₚₑₐₖ at +100 mV of individual cells from data shown in f (mean ± s.d.). *p<0.03, **p<0.002, one-way ANOVA with Tukey’s multiple comparison test. FIG. 2H shows representative action potential recordings from cardiomyocytes expressing WT KCNQ1-YFP (left) or G589D-YFP (right) + nano alone (red) or enDUB-O1 (blue). FIG. 2I shows quantification of action potential duration at 90% repolarization (APD90) (n = 11 - 13; mean ± s.d.). **p<0.0002, one-way ANOVA with Tukey’s multiple comparison test.

FIGS. 3A-3K show that enDUBs facilitate novel rescue of mutant CFTR channels in combination with Orkambi. FIG. 3A is a schematic of six CF patient mutations (Class II, VI) across the BBS-CFTR-YFP channel. Inset, Modular components of USP21 and enDUB-U21. FIG. 3B shows quantification of flow cytometry experiments for surface expression (BTX₆₄₇) of CFTR mutant channels in the presence of nano alone (black) or + lumacaftor (3 µM) (red), and enDUB-O1 alone (blue) or + lumacaftor (3 µM) (green), analyzed from YFP- and CFP-positive cells (n ≥ 5000 cells per experiment; N = 3; mean ± s.e.m). Data are normalized to values from the WT CFTR control group (dotted line). *p<0.02, **p<0.0001, two-way ANOVA followed by Dunnett’s test. FIG. 3C shows an exemplar family of basal, forskolin-activated (10 µM), and CFTRinh-172-treated (10 µM) WT CFTR currents from whole-cell patch clamp measurements in HEK293 cells. FIG. 3D shows population I-V curves for basal (black square, n = 16) and forskolin-activated (red square, n = 16) WT CFTR currents. FIGS. 3E-3G show an exemplar family of basal and forskolin-activated from untransfected (FIG. 3E); and 4326delTC (FIG. 3F); N1303K CFTR mutant (FIG. 3G) expressing cells. FIG. 3H shows an exemplar family of forskolin-activated, VX770-potentiated (5 µM) currents for 4236delTC mutant channels after 24 hr VX809 treatment (3 µM) and co-expression with nano (left) or enDUB-U21 (right). FIG. 3I shows population I-V curves for forskolin-activated, VX770-potentiated currents from 4326delTC mutants expressing nano (black circle, n = 17), versus VX809-treated 4326delTC cells expressing nano (red square, n =15) or enDUB-U21 (green triangle, n = 14). FIGS. 3J and 3K provide the same format as FIGS. 3H and 3J for N1303K mutant channels (n ≥ 8). **p<0.0001, two-way ANOVA with Tukey’s multiple comparison test.

FIGS. 4A-4G show that CF-targeted enDUB combination therapy functionally rescues common and rare trafficking-deficient CFTR mutations in FRT cells. FIG. 4A shows the structure of a full-length CFTR channel adapted from Liu, et al. 2017 (PDB: 5UAK). NBD1 highlighted in red. In FIG. 4B, Top, a schematic is shown for nanobody selection via yeast surface display library. Bottom, shows exemplary flow cytometry plots after MACS/FACS enrichment of yeast library with target binders (red). In FIG. 4C, Top, is shown a schematic for a FRET binding assay in HEK293 cells co-expressing Cerulean-nb.E3h (donor) and Venus-CFTR (acceptor). Bottom, shows flow cytometric FRET binding curves with FRET donor efficiency as function of free acceptor, with Cerulean-nb.E3h (blue) and Cerulean alone control (black) (n ≥ 10,000 cells per experiment; N = 2). FIG. 4D shows an exemplar family of forskolin-activated, VX770-potentiated currents in FRT cells stably expressing WT CFTR (left) or N1303K after 24 hr VX809 treatment and coexpressing either CFP alone (middle) or enDUB-U21_{CF.E3h} (right). FIG. 4E shows population I-V curves for forskolin-activated WT (black circle, n = 7) and N1303K (red square, n = 8) cells, compared to VX809-treated, forskolin-activated, and VX770-potentiated N1303K cells expressing CFP alone (green triangle, n = 12) or enDUB-U21_{CF.E3h} (blue triangle, n = 10). **p<0.0005, two-way ANOVA with Tukey’s multiple comparison test. FIG. 4F shows an exemplar family of forskolin-activated, VX770-potentiated currents in FRT cells stably expressing WT CFTR (left) or and F508del after 24 hr VX809 treatment and co-expressing either nb.T2a (middle) or enDUB-U21_{CF.E3h} (right). FIG. 4G shows population I-V curves for forskolin-activated WT (black circle, n = 7) and F508del (red square, n = 8) cells compared to VX809-treated, forskolin-activated, and VX770-potentiated N1303K cells expressing CFP alone (brown diamond, n = 8), nb.T2a (green triangle, n = 11), or enDUBU21_{CF.T2a} (blue triangle, n = 12). **p<0.005, two-way ANOVA with Tukey’s multiple comparison test.

FIGS. 5A-5C show that enDUB-O1 requires catalytic activity and target specificity for ubiquitin-dependent rescue of KCNQ1 channels. In FIG. 5A, (Left) a schematic is shown of an experimental strategy; BBS-Q1-YFP was co-transfected with nanobody alone (grey line), NEDD4L + nano (red line), or NEDD4L + enDUB-O1 (blue line). (Right) Cumulative distribution histograms of Alexa₆₄₇ fluorescence from flow cytometry analyses. Plot generated from population of YFP- and CFPpositive cells (n ≥ 5000 cells per experiment; N = 2). FIG. 5B shows the same experiment as in FIG. 5A, but using catalytically inactive enDUB-O1* with C320S. FIG. 5C shows the same experiment as in FIG. 5A, but with untagged BBS-Q1 co-expressed with enDUB-O1 as a control for target specificity.

FIGS. 6A-6B show that the ubiquitin status of the G589D LQT1 mutation is not enhanced compared to WT and V524G channels. FIG. 6A shows a Western blot of KCNQ1 pulldowns probed with anti-KCNQ1 antibody from HEK293 cells expressing WT, G589D, and V524G KCNQ1-YFP channels with nano alone (left) or enDUB-O1 (right) (representative of two independent experiments) . FIG. 6B shows anti-ubiquitin labeling of KCNQ1 pulldowns after stripping the Western blot from FIG. 6A.

FIGS. 7A-7E show that enDUB treatment rescues total KCNQ1 expression but not surface trafficking of N-terminal, ERAD-associated LQT1 mutations. FIG. 7A is schematic of two ERADassociated LQT1 patient mutations along the N-terminus of KCNQ1. FIG. 7B shows flow cytometry analyses of total Q1 expression (YFP fluorescence) in cells expressing WT BBS-KCNQ1-YFP + nanobody (left, control, black), and L114P mutant + nano (center, red) or enDUB-O1 (right, blue). FIG. 7C shows cumulative distribution histograms of YFP fluorescence for the experiment shown in FIG. 7B (left) and a similar experiment with Y111C KCNQ1 mutant (right). Plot generated from population of YFP- and CFP-positive cells (n ≥ 5000 cells per experiment; N = 2). FIGS. 7D and 7E show flow cytometry analyses and cumulative distribution histograms of surface Q1 expression (Alexa₆₄₇ fluorescence), using the same format as FIGS. 7B and 7C.

FIGS. 8A-8B show that enDUB-U21 has greater efficacy than enDUB-O1 in surface rescue of N1303K CFTR mutant channels. FIG. 8A shows cumulative distribution histograms of Alexa₆₄₇ fluorescence from flow cytometry analyses for cells expressing WT BBS-CFTR-YFP + nano (dotted line) and N1303K mutation co-expressing nano alone (red line), enDUB-O1 (cyan line), enDUB-U21 (blue line). Plot generated from population of YFP- and CFP-positive cells (n ≥ 5000 cells per experiment; N = 2). FIG. 8B shows the same experimental design as FIG. 8A but with 24 hour incubation of VX809 with nano (green line), enDUB-O1 (cyan line) and enDUB-U21 (blue line).

FIGS. 9A-9C show that enDUB-U21 requires catalytic activity and target specificity for ubiquitin-dependent rescue of CFTR mutants. In FIG. 9A, (Left) a schematic of an experimental strategy is shown; WT BBS-CFTR-YFP + nano (dashed line) or N1303K mutants co-transfected with nano (red line) or enDUB-U21 (blue line). Cumulative distribution histograms (middle) and quantification (right) of Alexa₆₄₇ fluorescence from flow cytometry analyses. Plots generated from population of YFP- and CFP-positive cells (n ≥ 5000 cells per experiment; N = 3; mean ± s.e.m). Data are normalized to values from the WT CFTR control group (dotted line). FIG. 9B shows the same experiment as in FIG. 9A, but using catalytically inactive enDUB-U21* with C221S. FIG. 9C shows the same experiment as in FIG. 9A, but with an mCherrytargeted nanobody, m-enDUB-U21, as a control for target specificity.

FIGS. 10A-10B show that enDUB-U21 increases functional rescue of 4326delTC CFTR mutant channels in combination with lumacaftor ± ivacaftor. FIG. 10A shows an exemplar family of basal (top, black), forskolin-activated (middle, red), and VX770-potentiated (bottom, green) currents for 4236delTC mutant channels after 24 hr VX809 treatment (3 µM) and co-expression with nano (left) or enDUB-U21 (right). FIG. 10B shows population I-V curves for basal (black square), forskolin-activated (red circle), and VX770-potentiated (green triangle) currents from 4326delTC mutants co-expressing nano alone (left; n = 15) or enDUB-U21 (right; n = 14).

FIGS. 11A-11B show that enDUB-U21 increases functional rescue of N1303K CFTR mutant channels in combination with lumacaftor ± ivacaftor. FIG. 11A shows an exemplar family of basal (top, black), forskolin-activated (middle, red), and VX770-potentiated (bottom, green) currents for N1303K mutant channels after 24 hr VX809 treatment (3 µM) and co-expression with nano (left) or enDUBU21 (right). FIG. 11B shows population I-V curves for basal (black square), forskolin-activated (red circle), and VX770-potentiated (green triangle) currents from N1303K mutants co-expressing nano alone (left; n = 9) or enDUB-U21 (right; n = 11).

FIGS. 12A-12B show the development of NBD1 binders from a yeast surface display nanobody library. FIG. 12A shows the on-yeast binding affinity measurements of 9 nanobody clones using serial dilutions of purified FLAG-NBD1. FIG. 12B shows the flow cytometric surface labeling assay and cumulative distribution histograms of WT CFTR surface density alone (dotted line) or when co-expressed with nanobody clones.

FIGS. 13A-13F show that enDUB-U21_{CF.E3h} functionally rescues distinct Class II and VI CF-causing mutations in HEK293 cells in combination with Orkambi. FIG. 13A is schematic of YFP sensor halide quenching assay. FIG. 13B shows exemplar traces showing YFP quenching in HEK293 cells expressing mCh (grey) or mCh-tagged 4326delTC mutants alone (red), and 4326delTC mutants treated with VX809 (green) or VX809 + enDUB-U21_{CF.E3h} (blue) after addition of forskolin and VX770. FIG. 13Cshows a summary of iodide influx rates (n = 9). **p<0.0001 versus 4326delTC, one-way ANOVA with Tukey’s multiple comparison test. FIGS. 13D and 13E show the same formats as FIGS. 13B and 13C for N1303K mutant channels (n = 8). **p<0.0001 versus N1303K, one-way ANOVA with Tukey’s multiple comparison test. FIG. 13F shows population I-V curves for basal (left), forskolin-activated (middle), and VX770-potentiated (right) currents from mCh-tagged WT CFTR channels (black circle, n = 41) or 4326delTC mutants treated with VX809 and co-expressing CFP alone (red square, n = 29), nb.E3h (green triangle, n = 9) or enDUB-U21_{CF.E3h} (blue triangle, n = 12). *p<00.02, **p<0.0001, two-way ANOVA with Tukey’s multiple comparison test.

FIGS. 14A-14C show that enDUB-U21_{CF.T2a} rescues trafficking and function of F508del mutant channels in HEK293 cells in combination with lumacaftor ± ivacaftor. FIG. 14A shows flow cytometric FRET binding curves with FRET donor efficiency as a function of free acceptor, with Cerulean-nb.T2a (green) and Cerulean alone control (black) (n ≥ 10,000 cells per experiment; N = 2). FIG. 14B shows quantification of flow cytometry experiments for surface expression (BTX₆₄₇) of F508del mutant channels in presence of CFP alone (red), enDUB-U21_{CF.E3h} (orange), nb.T2a (green), or enDUB-U21_{CF.T2a} (blue) with or without VX809 treatment (shaded or plain), analyzed from YFP- and CFP-positive cells (n ≥ 5000 cells per experiment; N = 4; mean ± s.e.m). Data are normalized to the WT CFTR control group and the dotted line represents F508del VX809 treatment alone. †p<0.05 versus CFP+VX809, **p<0.0002 versus all, one-way ANOVA with Tukey’s multiple comparison test. FIG. 14C shows population I-V curves for basal (left), forskolin-activated (middle), and VX770- potentiated (right) currents from mCh-tagged WT CFTR channels (black circle, n = 41) or F508del mutants treated with VX809 and co-expressing CFP alone (red square, n = 8), nb.T2a (green triangle, n = 10) or enDUB-U21_{CF.T2a} (blue triangle, n = 9). *p<0.05, **p<0.0001, two-way ANOVA with Tukey’s multiple comparison test.

FIG. 15A shows the underlying symptoms and current treatments for cystic fibrosis (CF). FIG. 15B is a schematic detailing the ubiquitin-dependent regulation of CFTR surface expression, stability, and function. Forward trafficking pathways highlighted in blue, and reverse trafficking pathways highlighted in red.

In FIG. 16A, Left, shows the structure of an exemplary protein target, CFTR. NBD1 highlighted in red. Right, Structure of stabilizing enzyme, DUB. In FIG. 16B, Top, a schematic is shown for nanobody selection via yeast surface display library. Bottom, flow cytometry plots after MACS/FACS enrichment with target binders (red). In FIG. 16C, Top, there is shown a nanobody-based proof-of-concept ReSTORx molecule, ReSTORAb, comprised of an ‘active’ component (DUB binder; blue) and ‘targeting’ component (NBD1 binder; orange). Bottom, shows FRET analysis and binding curves for each component. In FIG. 16D, Left, there is shown a schematic of CFTR surface labeling assay and co-expression of CFTR-targeted ReSTORAb. Right, shows flow cytometry plots from ReSTORAb rescue of mutant channels. FIG. 16E shows the same assay as in FIG. 16D with USP2 as the deubiquitinase. FIG. 16F further shows a similar assay as in FIG. 16D with presense of lumacaftor (VX-809).

FIG. 17 shows a schematic of an exemplary bivalent nanobodybased ReSTORAb.

FIG. 18 shows that the bivalent nanobody-based ReSTORAb is able to rescue long QT syndrome (LQTS) trafficking deficits.

### DETAILED DESCRIPTION OF THE DISCLOSURE

One embodiment of the present disclosure is a bivalent molecule comprising: a) a deubiquitinase (DUB) binder; b) a target binder; and c) a variable linker between the DUB binder and the target binder, wherein the DUB binder is selected from intracellular antibody fragments, scFvs, nanobodies, antibody mimetics, monobodies, DARPins, lipocalins, and targeting sequences.

In some embodiments, the DUB is endogenous. In some embodiments, the DUB is selected from the ubiquitin specific proteases (USP) family, the ovarian tumor proteases (OTU) family, the ubiquitin C-terminal hydrolases (UCH) family, the Josephin domain family (Josephin), the motif interacting with ubiquitin-containing novel DUB family (MINDY), and the JAB1/MPN/Mov34 metalloenzyme domain family (JAMM). In some embodiments, the DUB is USP21 or USP2.

In some embodiments, the DUB binder is selected from intracellular antibody fragments, scFvs, nanobodies, antibody mimetics, monobodies, DARPins, lipocalins, and targeting sequences. In some embodiments, the DUB binder is a nanobody. In some embodiments, the nanobody binds to a USP family member. In some embodiments, the nanobody binds to a USP2. In some embodiments, the nanobody binds to a USP21. In some embodiments, the nanobody comprises a sequence set forth as any one of SEQ ID NOs: 1 to 6. In some embodiments, the nanobody comprises: a) a complementarity determining region (CDR) 1 set forth as SEQ ID No: 7, a CDR2 set forth as SEQ ID No: 8, and a CDR3 set forth as SEQ ID No: 9; b) a CDR1 set forth as SEQ ID No: 10, a CDR2 set forth as SEQ ID No: 11, and a CDR3 set forth as SEQ ID No: 12; c) a CDR1 set forth as SEQ ID No: 13, a CDR2 set forth as SEQ ID No: 14, and a CDR3 set forth as SEQ ID No: 15; d) a CDR1 set forth as SEQ ID No: 16, a CDR2 set forth as SEQ ID No: 17, and a CDR3 set forth as SEQ ID No: 18; e) a CDR1 set forth as SEQ ID No: 19, a CDR2 set forth as SEQ ID No: 20, and a CDR3 set forth as SEQ ID No: 21; or f) a CDR1 set forth as SEQ ID No: 22, a CDR2 set forth as SEQ ID No: 23, and a CDR3 set forth as SEQ ID No: 24.

In some embodiments, aberrant ubiquitination of the target to which the target binder binds causes a disease. In some embodiments, the disease is an inherited ion channelopathy. As used herein, the term “inherited ion channelopathy” refers to rare diseases that encompass a broad range of disorders in the nervous system, cardiovascular system, respiratory system, endocrine system, and urinary system. In the present disclosure, an “inherited ion channelopathy” includes but is not limited to: epilepsy, migraine, neuropathic pain, cardiac arrhythmias, long QT syndrome, Brugada syndrome, cystic fibrosis, diabetes, hyperinsulinemic hypoglycemia, Bartter syndrome, and diabetes insipidus. In some embodiments, the disease is long QT syndrome. In some embodiments, the disease is cystic fibrosis.

In some embodiments, the target to which the target binder binds is cystic fibrosis transmembrane conductance regulator (CFTR).

In some embodiments, the target binder is selected from intracellular antibody fragments, scFvs, nanobodies, antibody mimetics, monobodies, DARPins, lipocalins, and targeting sequences. In some embodiments, the target binder is a nanobody. In some embodiments, the nanobody binds to NBD1 domain of cystic fibrosis transmembrane conductance regulator (CFTR). In some embodiments, the nanobody comprises a sequence set forth as any one of SEQ ID NOs: 25 to 38. In some embodiments, the nanobody comprises: a) a complementarity determining region (CDR) 1 set forth as SEQ ID No: 39, a CDR2 set forth as SEQ ID No: 40, and a CDR3 set forth as SEQ ID No: 41; b) a CDR1 set forth as SEQ ID No: 42, a CDR2 set forth as SEQ ID No: 43, and a CDR3 set forth as SEQ ID No: 44; c) a CDR1 set forth as SEQ ID No: 45, a CDR2 set forth as SEQ ID No: 46, and a CDR3 set forth as SEQ ID No: 47; d) a CDR1 set forth as SEQ ID No: 48, a CDR2 set forth as SEQ ID No: 49, and a CDR3 set forth as SEQ ID No: 50; e) a CDR1 set forth as SEQ ID No: 51, a CDR2 set forth as SEQ ID No: 52, and a CDR3 set forth as SEQ ID No: 53; f) a CDR1 set forth as SEQ ID No: 54, a CDR2 set forth as SEQ ID No: 55, and a CDR3 set forth as SEQ ID No: 56; g) a CDR1 set forth as SEQ ID No: 57, a CDR2 set forth as SEQ ID No: 58, and a CDR3 set forth as SEQ ID No: 59; h) a CDR1 set forth as SEQ ID No: 60, a CDR2 set forth as SEQ ID No: 61, and a CDR3 set forth as SEQ ID No: 62; i) a CDR1 set forth as SEQ ID No: 63, a CDR2 set forth as SEQ ID No: 64, and a CDR3 set forth as SEQ ID No: 65; j) a CDR1 set forth as SEQ ID No: 66, a CDR2 set forth as SEQ ID No: 67, and a CDR3 set forth as SEQ ID No: 68; k) a CDR1 set forth as SEQ ID No: 69, a CDR2 set forth as SEQ ID No: 70, and a CDR3 set forth as SEQ ID No: 71; l) a CDR1 set forth as SEQ ID No: 72, a CDR2 set forth as SEQ ID No: 73, and a CDR3 set forth as SEQ ID No: 74; m) a CDR1 set forth as SEQ ID No: 75, a CDR2 set forth as SEQ ID No: 76, and a CDR3 set forth as SEQ ID No: 77; or n) a CDR1 set forth as SEQ ID No: 78, a CDR2 set forth as SEQ ID No: 79, and a CDR3 set forth as SEQ ID No: 80.

In some embodiments, the linker is an alkyl, a polyethylene glycol (PEG) or other similar molecule, or a click linker. As used herein, the “alkyl” may be branched or linear, substituted or unsubstituted. The length of the alkyl is selected to maximize, or at least not substantially interfere with the efficient binding of the DUB binder and the target binder. For example, the “alkyl” may be C₁-C₂₅, such as C₁-C₂₀, including C₁-C₁₅, C₁-C₁₀ and C₁-C₅. Thus, the alkyl linker may include C1, C2, C3, C4, C5, C6, C7, C8, C9, C10, C11, C12, C13, C14, C15, C16, C17, C18, C19, C20, C21, C22, C23, C24, C25 or higher carbon chain. As used herein a “click linker” is a class of biocompatible small molecules that are used in bioconjugation, allowing the joining of substrates of choice with specific biomolecules. It is based on “click” chemistry which is fully desctribed in Kolb et al. (2001) “Click Chemistry: Diverse Chemical Function from a Few Good Reactions”. Angewandte Chemie International Edition. 40 (11): 2004-2021.

Another embodiment of the present disclosure is a method of treating or ameliorating the effects of a disease in a subject, comprising administering to the subject an effective amount of a bivalent molecule disclosed herein.

In some embodiments, the subject is human. In some embodiments, the disease is selected from the group consisting of an inherited ion channelopathy, a cancer, a cardiovascular condition, an infectious disease, and a metabolic disease. In some embodiments, the inherited ion channelopathy is selected from the group consisting of epilepsy, migraine, neuropathic pain, cardiac arrhythmias, long QT syndrome, Brugada syndrome, cystic fibrosis, diabetes, hyperinsulinemic hypoglycemia, Bartter syndrome, and diabetes insipidus. In some embodiments, the inherited ion channelopathy is cystic fibrosis.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population may fail to respond or respond inadequately to treatment.

As used herein, the terms “ameliorate”, “ameliorating” and grammatical variations thereof mean to decrease the severity of the symptoms of a disease in a subject, preferably a human.

As used herein, “administration,” “administering” and variants thereof means introducing a composition, such as a synthetic membrane-receiver complex, or agent into a subject and includes concurrent and sequential introduction of a composition or agent. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. A suitable route of administration allows the composition or the agent to perform its intended function. For example, if a suitable route is intravenous, the composition is administered by introducing the composition or agent into a vein of the subject. Administration can be carried out by any suitable route.

As used herein, a “subject” is a mammal, preferably, a human. In addition to humans, categories of mammals within the scope of the present disclosure include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc.

Another embodiment of the present disclosure is a method of identifying and preparing a nanobody binder targeting a protein of interest, comprising: a) constructing a naive yeast library that expresses synthetic nanobodies; b) incubating the naive yeast library with the protein of interest; c) selecting yeast cells expressing nanobodies that bind to the protein of interest by magnetic-activated cell sorting (MACS); d) amplifying the selected cells and constructing an enriched yeast library; e) incubating the enriched yeast library with the protein of interest; f) selecting yeast cells expressing nanobodies that bind to the protein of interest by fluorescence activated cell sorting (FACS); g) amplifying the selected cells and constructing a further enriched yeast library; h) repeating steps e) to g) twice; and i) sorting the selected yeast cells as single cells and cultivating as monoclonal colonies for binding validation and plasmid isolation.

In some embodiments, the protein of interest is cystic fibrosis transmembrane conductance regulator (CFTR). In some embodiments, the protein of interest is a deubiquitinase (DUB).

### Additional Definitions

The term “amino acid” means naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. An “amino acid analog” means compounds that have the same basic chemical structure as a naturally occurring amino acid, e.g., a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Imino acids such as, e.g., proline, are also within the scope of “amino acid” as used here. An “amino acid mimetic” means a chemical compound that has a structure that is different from the general chemical structure of an amino acid, but that functions similarly to a naturally occurring amino acid.

As used herein, the terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymers.

“Nucleic acid” or “oligonucleotide” or “polynucleotide” used herein means at least two nucleotides covalently linked together. Many variants of a nucleic acid may be used for the same purpose as a given nucleic acid. Thus, a nucleic acid also encompasses substantially identical nucleic acids and complements thereof.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods.

The nucleic acid may also be an RNA such as an mRNA, tRNA, short hairpin RNA (shRNA), short interfering RNA (siRNA), double-stranded RNA (dsRNA), transcriptional gene silencing RNA (ptgsRNA), Piwi-interacting RNA, pri-miRNA, pre-miRNA, micro-RNA (miRNA), or anti-miRNA.

As used herein, the term “antibody” encompasses an immunoglobulin whether natural or partly or wholly synthetically produced, and fragments thereof. The term also covers any protein having a binding domain which is homologous to an immunoglobulin binding domain. These proteins can be derived from natural sources, or partly or wholly synthetically produced. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies; murine antibodies; chimeric, mouse-human, mouse-primate, primate-human monoclonal antibodies, anti-idiotype antibodies, antibody fragments, such as, e.g., scFv, (scFv)2, Fab, Fab′, and F(ab′)2, F(ab1)2, Fv, dAb, and Fd fragments, diabodies, nanobodies and antibody-related polypeptides. Antibody includes bispecific antibodies and multispecific antibodies so long as they exhibit the desired biological activity or function.

The term “antigen binding fragment” used herein refers to fragments of an intact immunoglobulin, and any part of a polypeptide including antigen binding regions having the ability to specifically bind to the antigen. For example, the antigen binding fragment may be a F(ab′)2 fragment, a Fab′ fragment, a Fab fragment, a Fv fragment, or a scFv fragment, but is not limited thereto. A Fab fragment has one antigen binding site and contains the variable regions of a light chain and a heavy chain, the constant region of the light chain, and the first constant region CH1 of the heavy chain. A Fab′ fragment differs from a Fab fragment in that the Fab′ fragment additionally includes the hinge region of the heavy chain, including at least one cysteine residue at the C-terminal of the heavy chain CH1 region. The F(ab′)2 fragment is produced whereby cysteine residues of the Fab′ fragment are joined by a disulfide bond at the hinge region. A Fv fragment is the minimal antibody fragment having only heavy chain variable regions and light chain variable regions, and a recombinant technique for producing the Fv fragment is well known in the art. Two-chain Fv fragments may have a structure in which heavy chain variable regions are linked to light chain variable regions by a non-covalent bond. Single-chain Fv (scFv) fragments generally may have a dimer structure as in the two-chain Fv fragments in which heavy chain variable regions are covalently bound to light chain variable regions via a peptide linker or heavy and light chain variable regions are directly linked to each other at the C-terminal thereof. The antigen binding fragment may be obtained using a protease (for example, a whole antibody is digested with papain to obtain Fab fragments, and is digested with pepsin to obtain F(ab′)2 fragments), and may be prepared by a genetic recombinant technique. A dAb fragment consists of a VH domain. Single-chain antibody molecules may comprise a polymer with a number of individual molecules, for example, dimmer, trimer or other polymers.

“Vector” used herein refers to an assembly which is capable of directing the expression of desired protein. The vector must include transcriptional promoter elements which are operably linked to the gene(s) of interest. The vector may be composed of either deoxyribonucleic acids (“DNA”), ribonucleic acids (“RNA”), or a combination of the two (e.g., a DNA-RNA chimeric). Optionally, the vector may include a polyadenylation sequence, one or more restriction sites, as well as one or more selectable markers such as neomycin phosphotransferase or hygromycin phosphotransferase. Additionally, depending on the host cell chosen and the vector employed, other genetic elements such as an origin of replication, additional nucleic acid restriction sites, enhancers, sequences conferring inducibility of transcription, and selectable markers, may also be incorporated into the vectors described herein.

As used herein, the terms “cell”, “host cell” or “recombinant host cell” refers to host cells that have been engineered to express a desired recombinant protein. Methods of creating recombinant host cells are well known in the art. For example, see Sambrook et al. (MOLECULAR CLONING: A LABORATORY MANUAL (Sambrook et al, eds., Cold Spring Harbor Laboratory Press, Cold Spring Harbor, 1989), Ausubel et al. (CURRENT PROTOCOLS IN MOLECULAR BIOLOGY Ausubel et al., eds., John Wiley & Sons, New York, 1987). In the present disclosure, the host cells are transformed with the vectors described herein.

Recombinant host cells as used herein may be any of the host cells used for recombinant protein production, including, but not limited to, bacteria, yeast, insect and mammalian cell lines.

As used herein, the term “increase,” “enhance,” “stimulate,” and/or “induce” (and like terms) generally refers to the act of improving or increasing, either directly or indirectly, a concentration, level, function, activity, or behavior relative to the natural, expected, or average, or relative to a control condition.

As used herein, the term “inhibit,” “suppress,” “decrease,” “interfere,” and/or “reduce” (and like terms) generally refers to the act of reducing, either directly or indirectly, a concentration, level, function, activity, or behavior relative to the natural, expected, or average, or relative to a control condition.

The terminology used herein is for the purpose of describing particular embodiments only and is not intended to be limiting. As used in the specification and the appended claims, the singular forms “a,” “an,” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The following examples are provided to further illustrate certain aspects of the present disclosure. These examples are illustrative only and are not intended to limit the scope of the disclosure in any way.

### EXAMPLES

### Example 1

### Targeted Deubiquitination Rescues Trafficking-Deficient Ion Channelopathies

Inherited or de novo mutations in ion channels underlie diverse diseases (termed ion channelopathies) including cardiac arrhythmias, epilepsy, and cystic fibrosis (Kullmann, 2010; Bohnen et al. 2016; Cutting, 2014). Impaired channel trafficking to the cell surface underlies many distinct ion channelopathies (Curran and Mohler, 2015), a shared mechanism that represents an as-yet-unexploited opportunity to develop a common strategy to treat dissimilar rare diseases. Ubiquitination is a prevalent post-translational modification that in ion channels limits their surface density by inhibiting forward trafficking, enhancing endocytosis, and promoting degradation (Foot et al. 2017; MacGurn et al. 2012). Here, we show that targeted deubiquitination can rescue distinct disease-causing trafficking-deficient mutant ion channels. We developed engineered deubiquitinases (enDUBs), featuring nanobodies fused to minimal deubiquitinase catalytic components, that enable selective removal of ubiquitin chains from target channels (Table 1). This targeted deubiquitination approach successfully rescued surface trafficking and functional currents of different mutant ion channels - KCNQ1 and cystic fibrosis transmembrane regulator (CFTR) - that cause long QT syndrome (LQT1) and cystic fibrosis (CF), respectively. In a guinea pig ventricular cardiomyocyte model of LQT1, enDUB treatment rescued slow delayed rectifier K⁺ currents and normalized action potential duration. Further, CFTR-targeted enDUBs displayed remarkable synergy in the functional rescue of CF mutations when combined with the FDA-approved therapy, Orkambi. Thus, we introduce targeted deubiquitination as a powerful general approach to classify and rescue diverse diseases for which impaired ion channel trafficking to the cell surface is the primary mechanism.

Ion channelopathies resulting from inherited or de novo mutations in ion channels underlie various diseases spanning the nervous (epilepsy, migraine, neuropathic pain) (Kullmann, 2010), cardiovascular (long QT syndrome, Brugada syndrome) (Bohnen et al. 2016), respiratory (cystic fibrosis) (Cutting, 2014), endocrine (diabetes, hyperinsulinemic hypoglycemia) (Ashcroft and Rorsman, 2013), and urinary (Bartter syndrome, diabetes insipidus) systems (Imbrici et al. 2016). Hundreds of disease-causing mutations are typically found in individual ion channels, and present an extraordinary challenge for treatment. Mechanism-based approaches to correct underlying abnormalities that can be generally applied across different ion channels would be advantageous, but are lacking (Imbrici et al. 2016; Wulff et al. 2019).

Long QT syndrome type 1 (LQT1) and cystic fibrosis (CF) arise from loss-of-function mutations in KCNQ1 (Kv7.1) (Bohnen et al. 2016; Tester et al. 2005) and cystic fibrosis transmembrane regulator (CFTR) (Cutting, 2014) channels, respectively. LQT1 increases the risk of exertion triggered cardiac arrhythmias and sudden cardiac death, while CF patients display impaired mucus clearance from the airways leading to recurrent bacterial infection, uncontrolled inflammation, lung damage, and low life expectancy. For both KCNQ1 and CFTR, a prominent mechanism underlying loss-of-function of many mutations is impaired channel trafficking to the surface (Wilson et al. 2005; Haardt et al. 1999; Cheng et al. 1990). The present disclosure exploited this shared mechanism to develop an approach that is amenable to therapeutic development and can be applied to diverse ion channels.

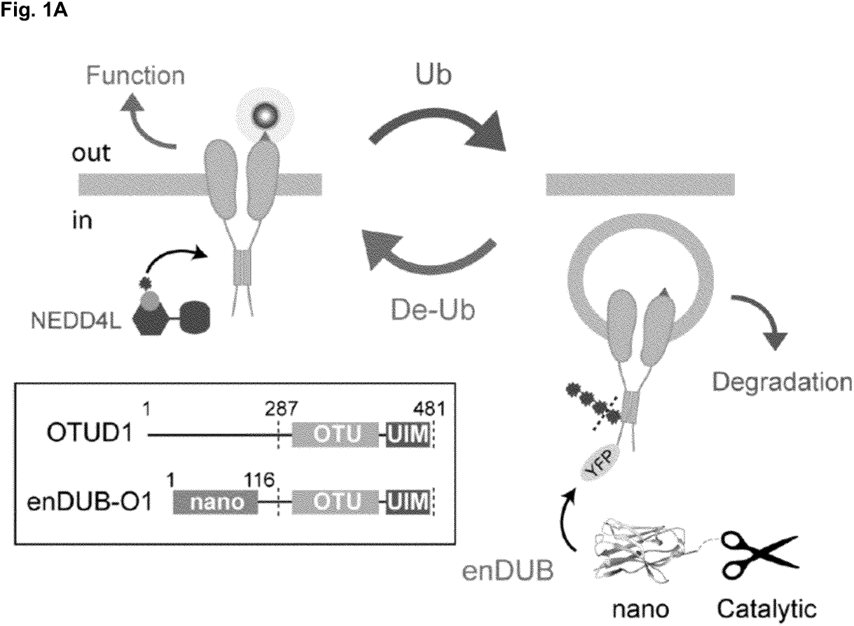

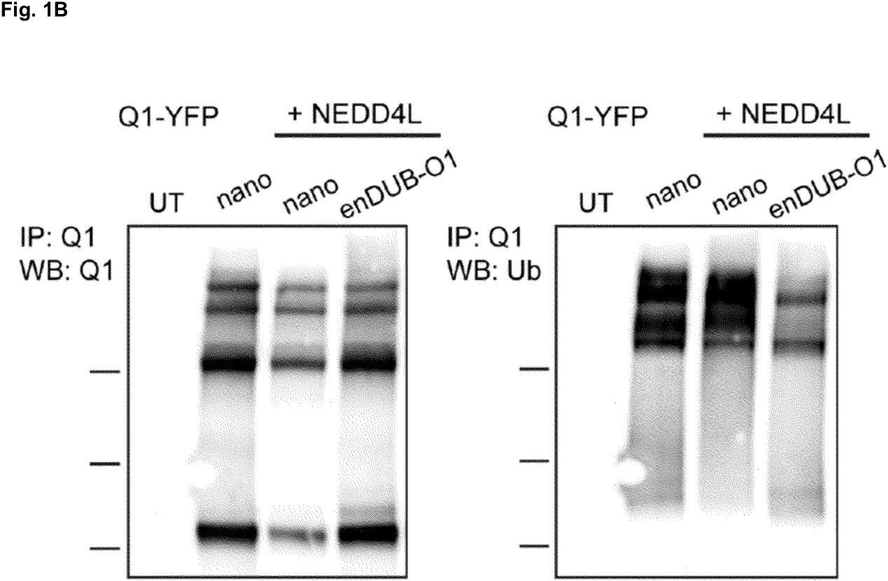

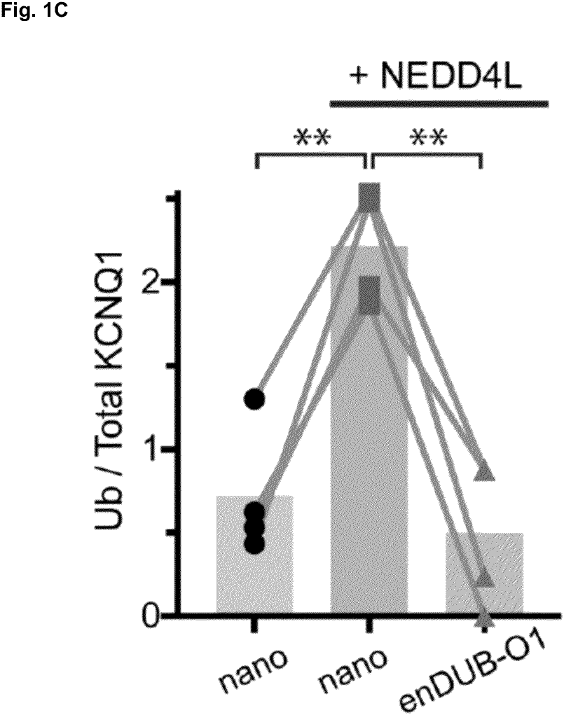

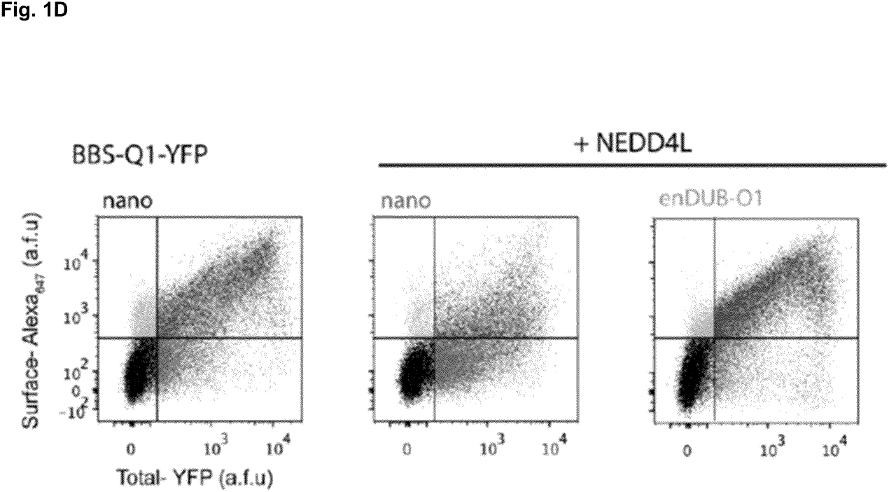

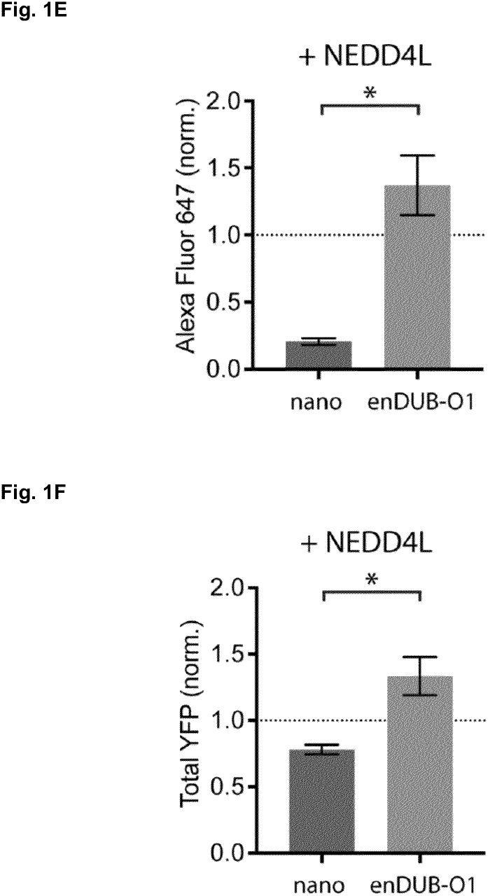

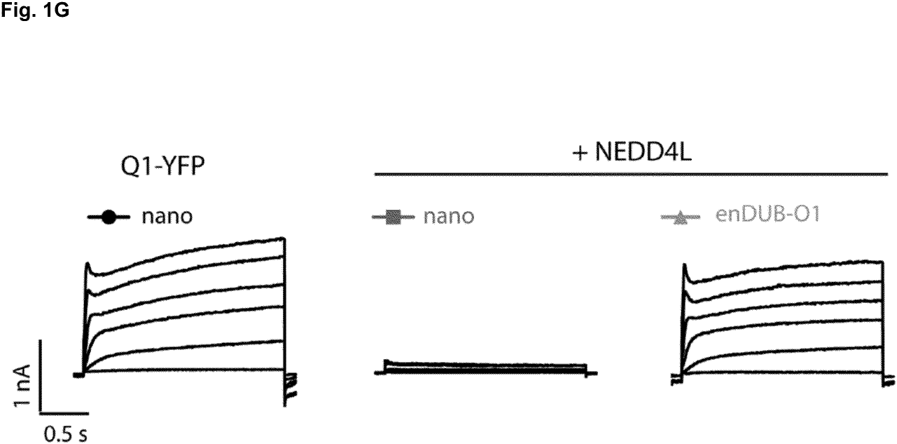

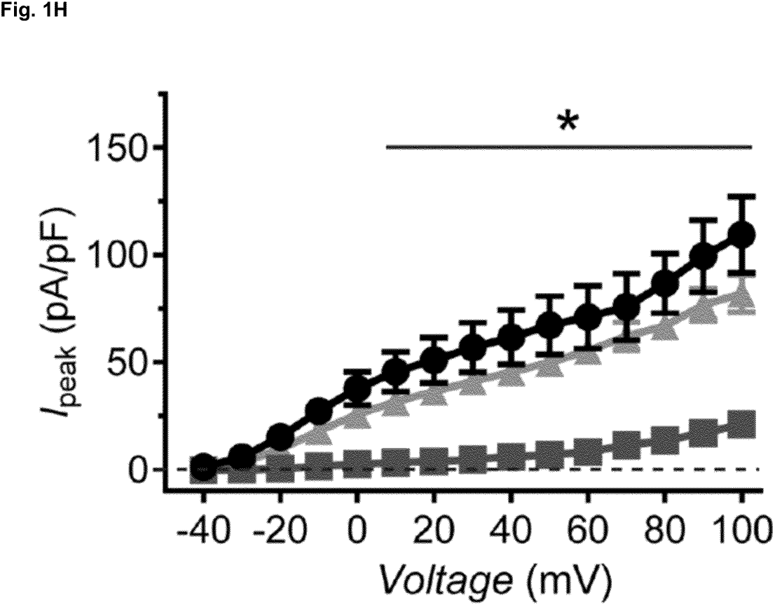

Because ubiquitination/deubiquitination is a primary determinant of the surface density of ion channels (FIG. 1A), it is hypothesized that removing ubiquitin from mutant channels would rescue trafficking-deficient ion channels. Since ubiquitination is a widespread physiological phenomenon, the goal is to develop a targeted deubiquitination approach that would circumvent problematic off-target effects generally associated with targeting the ubiquitin/proteasomal system (Nalepa et al. 2006; Huang and Dixit, 2016). Initially, we utilized YFP-tagged KCNQ1, a K⁺ ion channel known to be down-regulated at the protein and functional levels by NEDD4L, an E3 ubiquitin ligase (Jespersen et al. 2007). We developed a YFP-targeted engineered deubiquitinase (enDUB-O1) by fusing the minimal catalytic unit of ovarian tumor deubiquitinase 1 (OTUD1), a deubiquitinase with intrinsic preference for hydrolysis of K63 polyubiquitin chains (Mevissen et al. 2013), to a nanobody specific for GFP/YFP but not CFP18 (FIG. 1A, inset). We tested the efficacy and selectivity of enDUB-O1 using biochemical and functional assays in transiently transfected HEK293 cells (FIGS. 1B-1H).

Immunoprecipitation experiments in control cells expressing KCNQ1-YFP and anti-GFP nanobody (nano) showed robust expression of ubiquitinated KCNQ1 channels, reflecting endogenous E3 ligase activity (FIGS. 1B and 1C). Co-expressing NEDD4L with KCNQ1-YFP and nano resulted in decreased KCNQ1 levels (FIG. 1B, left), but increased ubiquitin signal (FIGS. 1B and 1C). In the presence of NEDD4L, enDUB-O1 rescued KCNQ1 expression, and prevented the increase in channel ubiquitination (FIGS. 1B and 1C).

A flow cytometry assay was performed to simultaneously measure KCNQ1-YFP total expression and surface density, and to assess the ability of enDUB-O1 (expressed in a 1:1 ratio with CFP using a P2A self-cleaving peptide plasmid) to antagonize the impact of NEDD4L on these two indices. NEDD4L significantly decreased KCNQ1 surface density (assessed by fluorescent bungarotoxin binding to an extracellular epitope tag) and total expression (assessed by YFP fluorescence), and both effects were reversed by enDUB-O1 (FIGS. 1D-1F). Catalytically dead enDUB-O1* did not rescue KCNQ1-YFP surface density, demonstrating DUB enzymatic activity is necessary for this effect (FIG. 5B). Moreover, enDUB-O1 did not rescue surface density of KCNQ1 channels lacking a YFP tag, confirming specificity of the targeted enDUB approach (FIG. 5C).

Whole-cell patch clamp electrophysiology was used to determine functionality of enDUB-O1-rescued KCNQ1 channels. Control cells expressing KCNQ1 + nano displayed robust KCNQ1 currents that were abolished with NEDD4L expression (FIGS. 1G and 1H); co-expressing enDUB-O1 fully rescued KCNQ1 currents (FIGS. 1G and 1H), confirming the efficacy of targeted deubiquitination with enDUBs to specifically deubiquitinate and stabilize functional channels of interest at the cell surface.

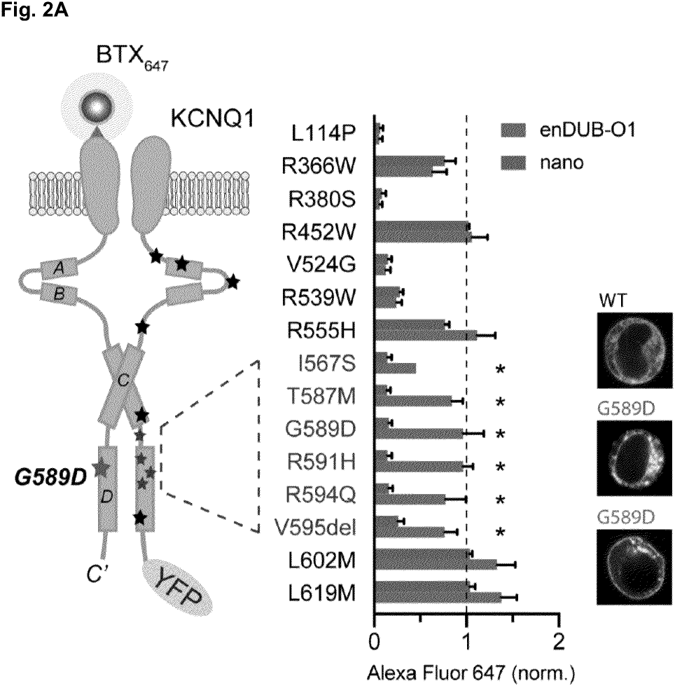

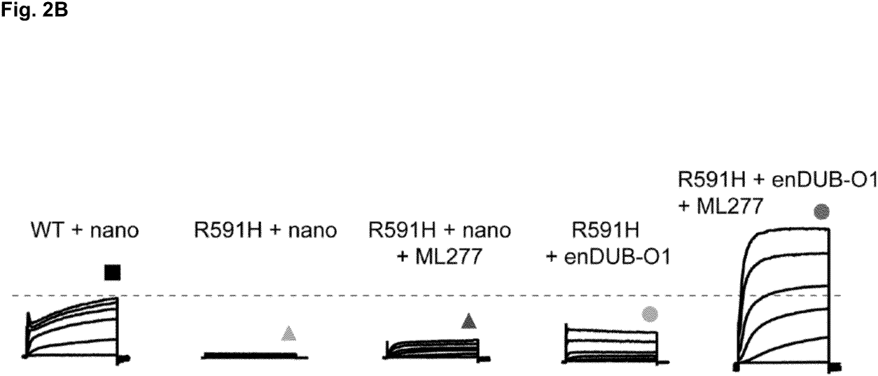

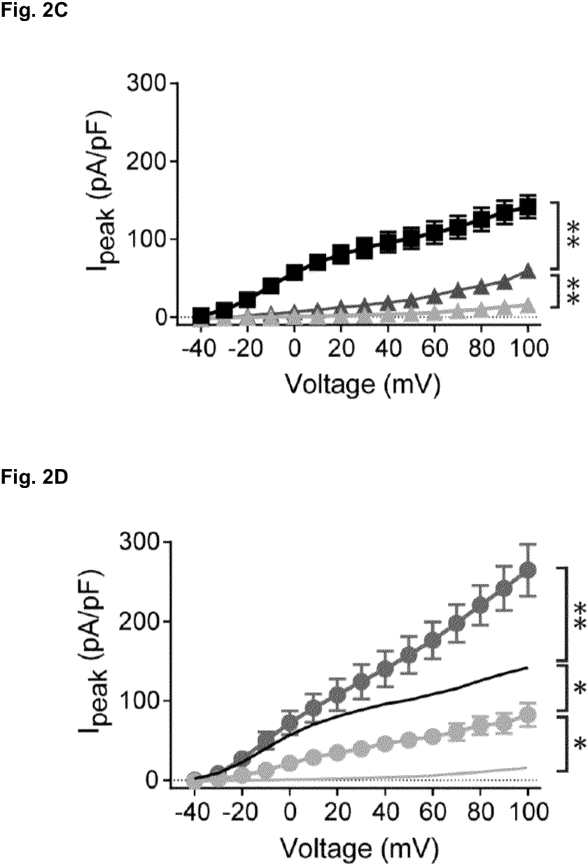

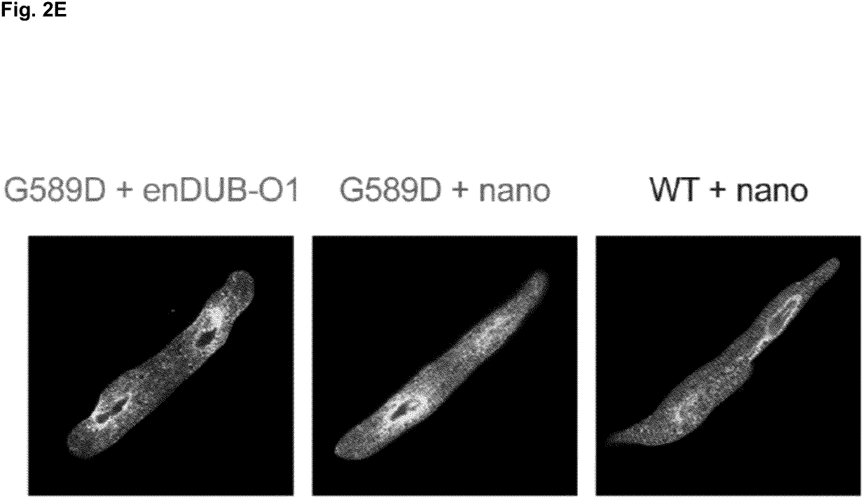

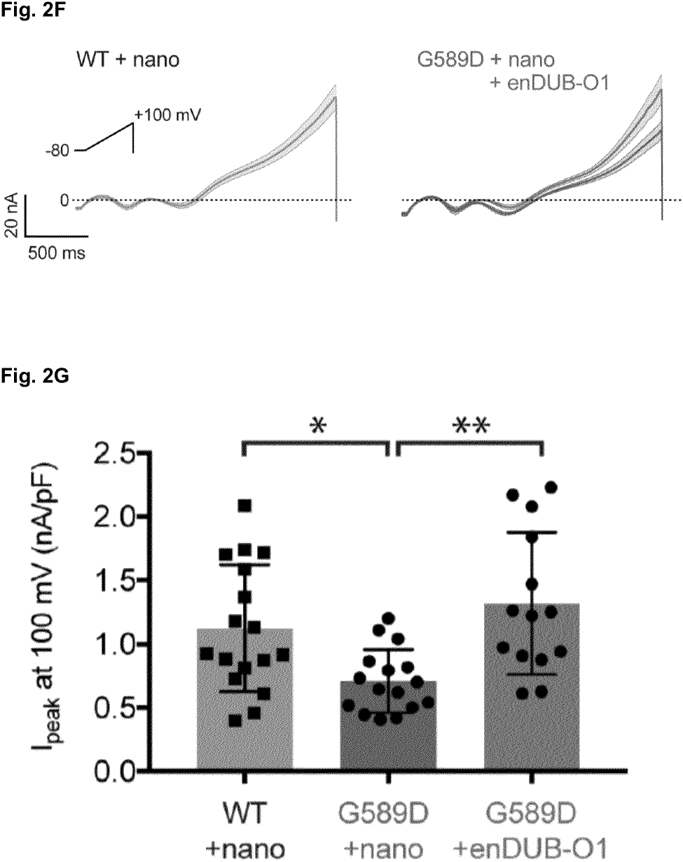

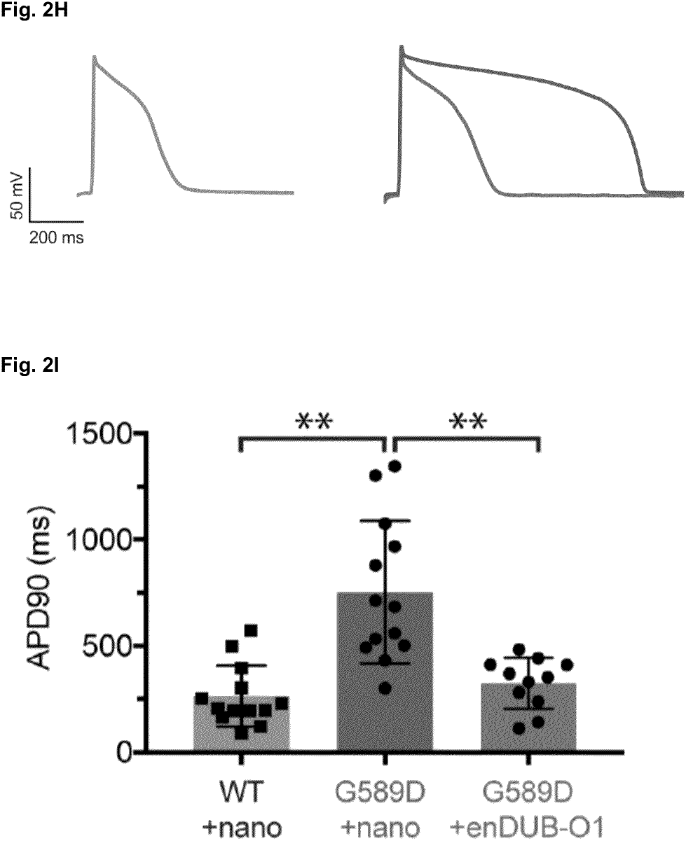

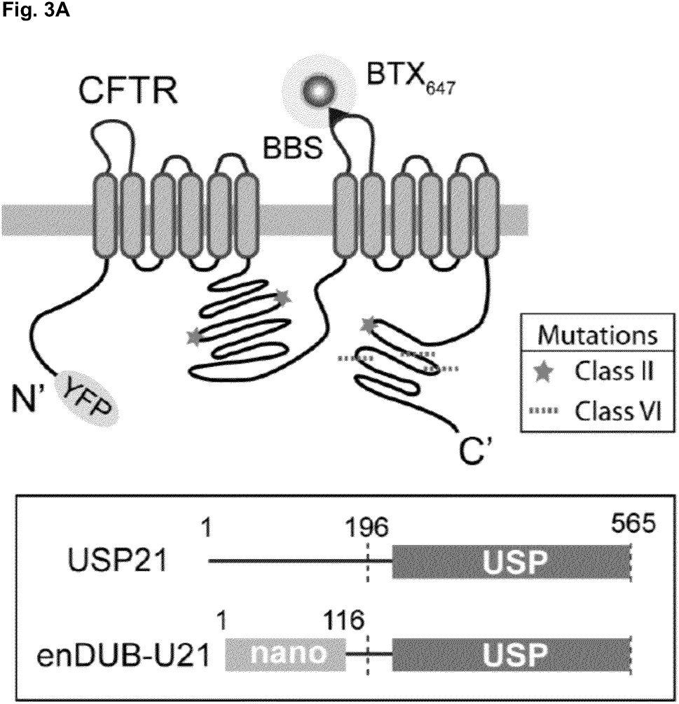

The next question is whether enDUBs would rescue trafficking-deficient mutant KCNQ1 channels that underlie LQT1. We used flow cytometry to determine the impact of 14 distinct LQT1 mutations in KCNQ1 C-terminus (Tester et al. 2005; Aromolaran et al. 2014), and a previously described endoplasmic reticulum-associated degradation (ERAD)-dependent mutation in the Nterminus (L114P) (Peroz et al. 2009), on channel surface density (FIG. 2A). In addition to L114P, 9 of the 14 C-terminus mutations showed significantly reduced surface trafficking compared to WT KCNQ1 (FIG. 2A, red bars). Remarkably, the surface density of 6 mutant channels was either partially or fully rescued with enDUB-O1 co-expression (FIG. 2A, blue bars and inset). The responsive mutant channels were clustered along the KCNQ1 coiled-coil tetramerization domain (helix D), defining a spatial ‘hotspot’ amenable to enDUBmediated rescue of trafficking (FIG. 2A, purple text).

Functionally, homotetrameric R591H channels displayed dramatically reduced currents compared to WT KCNQ1, consistent with their impaired surface trafficking (FIGS. 2B and 2C). Application of the KCNQ1 activator, ML277 (Mattmann et al. 2012) (1 µM), modestly increased R591H currents, implying a small fraction of channels at the surface (FIGS. 2B and 2C). Co-expressing enDUB-O1 significantly rescued R591H currents to approximately 50% of WT KCNQ1, which in light of the full rescue in surface density (FIG. 2A), suggests the mutation causes an additional impairment in either open probability or conductance (FIGS. 2B and 2D). Strikingly, ML277 markedly increased enDUB-O1-rescued R591H current amplitude to beyond WT KCNQ1 levels (FIGS. 2B and 2D).

LQT1 is typically inherited in an autosomal dominant fashion wherein patients possess one WT and one mutant allele (Bohnen et al. 2016). Accordingly, we next sought to recapitulate the heterotetrameric essence of LQT1 in cardiomyocytes from a species in which I_{Ks} is important for cardiac action potential repolarization. We used adenovirus to express YFPtagged WT or LQT1 mutant KCNQ1 channels in isolated adult guinea pig cardiomyocytes (FIG. 2E). Compared to cardiomyocytes expressing WT KCNQ1, those with G589D displayed reduced late outward current measured by slow voltage ramps to +100 mV (FIGS. 2F and 2G), and markedly prolonged action potential duration (APD) (FIGS. 2H and 2I), a characteristic feature of LQTS. Remarkably, enDUB-O1 treatment of G589D-expressing cardiomyocytes restored I_{Ks} and APD to WT KCNQ1 levels (FIGS. 2F-2I).

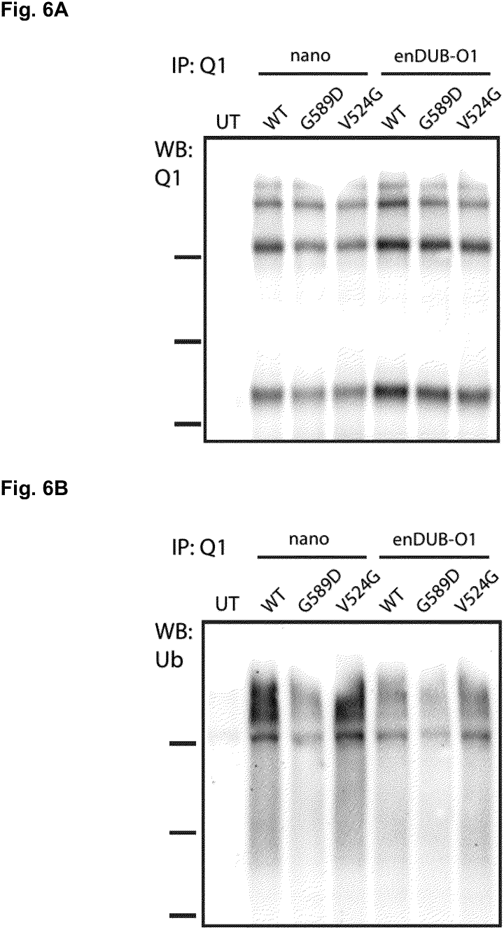

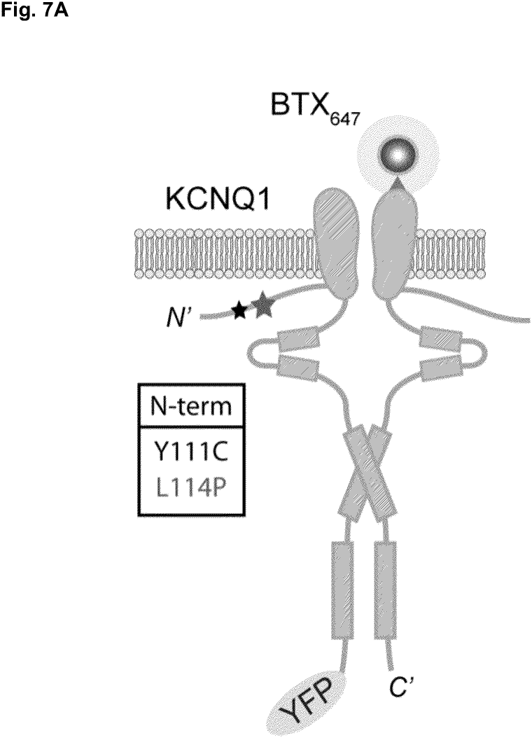

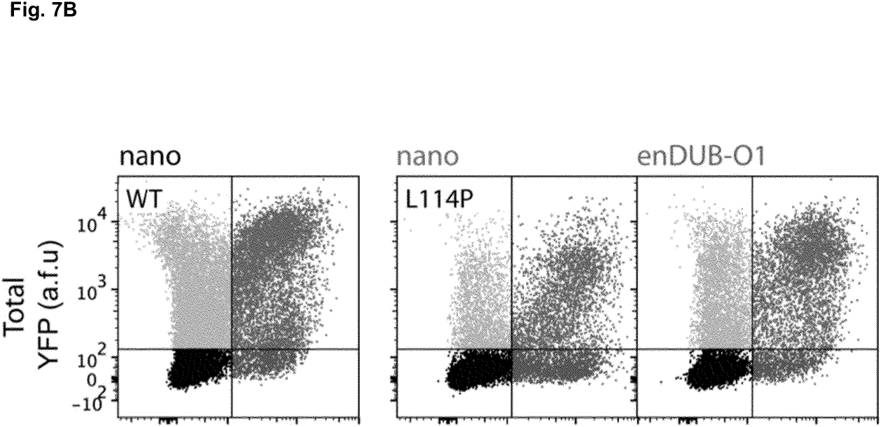

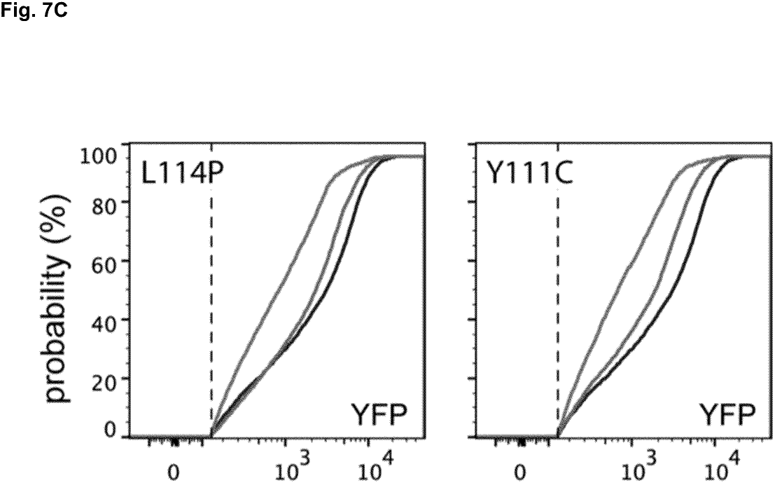

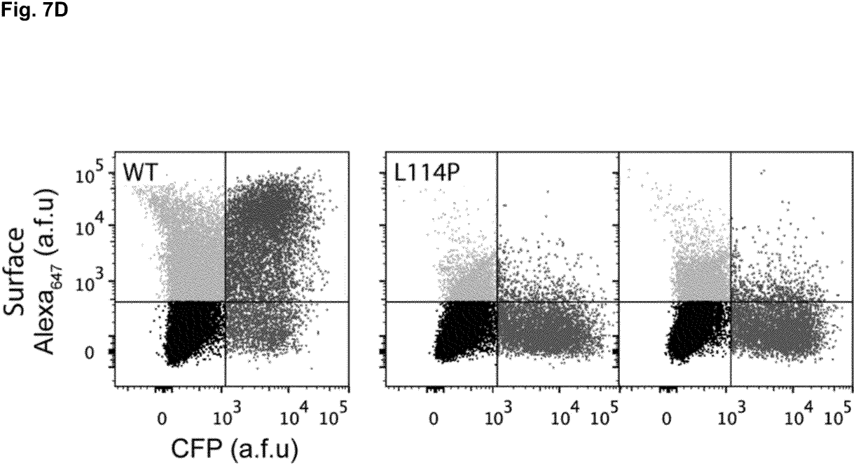

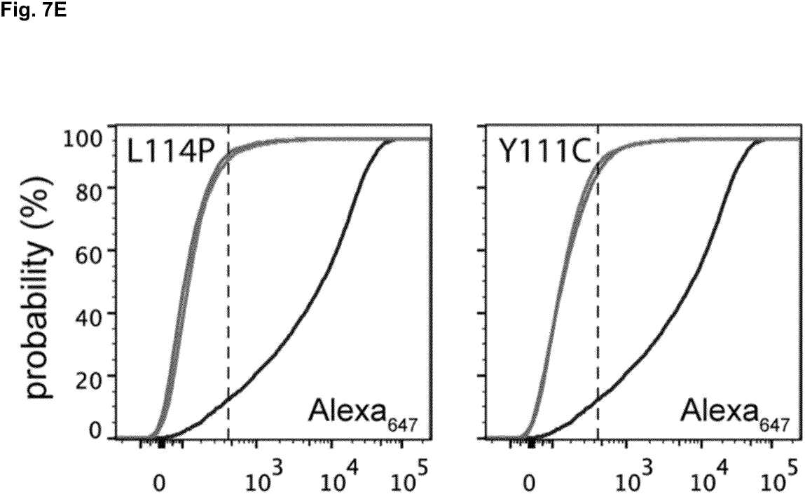

Notably, the amenability of a mutant KCNQ1 to enDUB-O1-mediated rescue was not simply correlated with the level of channel ubiquitination¾ for example, the baseline ubiquitin signal of G589D was less than that observed with V524G, a mutation not rescued by enDUB-O1 (FIGS. 6A-6B). Furthermore, enDUB-O1 rescued total protein expression but not surface density of ERAD-sensitive L114P and Y111C (FIGS. 7A-7E), suggesting additional mechanisms that prevent forward trafficking of misfolded proteins irrespective of their ubiquitination status.

To determine if enDUBs can similarly rescue functional channels in a different ion channelopathy for which impaired trafficking is a primary underlying cause, we turned to cystic fibrosis (CF) a devastating monogenic disease arising from loss-of-function mutations in CFTR, a Cl⁻ ion channel. Over 2000 distinct CF mutations have been mapped to CFTR, many of which reduce channel surface density due to due to impaired folding/trafficking (class II) or decreased plasma membrane stability (class VI) (Veit et al. 2016; Boeck and Amaral, 2016). Discovery of pharmacologic chaperones (correctors) and gating modifiers (potentiators) from high throughput screening has led to an FDA-approved combination therapy, Orkambi, consisting of lumacaftor (VX809; corrector) and ivacaftor (VX770; potentiator), for treating homozygous F508del mutations (Wainwright et al. 2015; Goor et al. 2011; Goor et al. 2009). Nevertheless, the clinical efficacy of Orkambi is often sub-optimal (~3% change in forced expiratory volume) and a substantial number of CFTR mutations are refractory to treatment, emphasizing an urgent need to develop complementary therapies (Boeck and Amaral, 2016; Farinha and Matos, 2016).

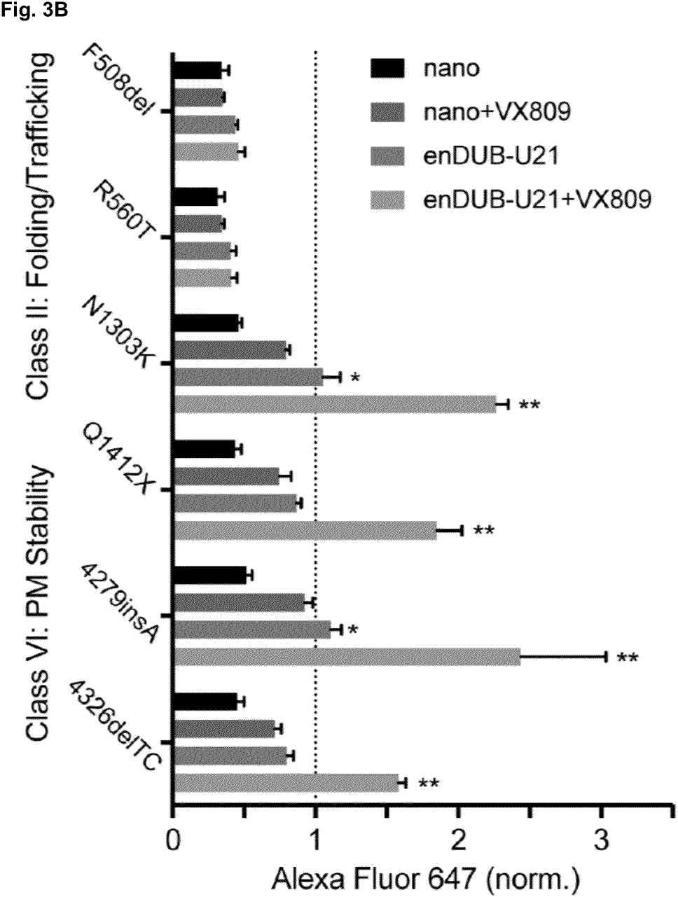

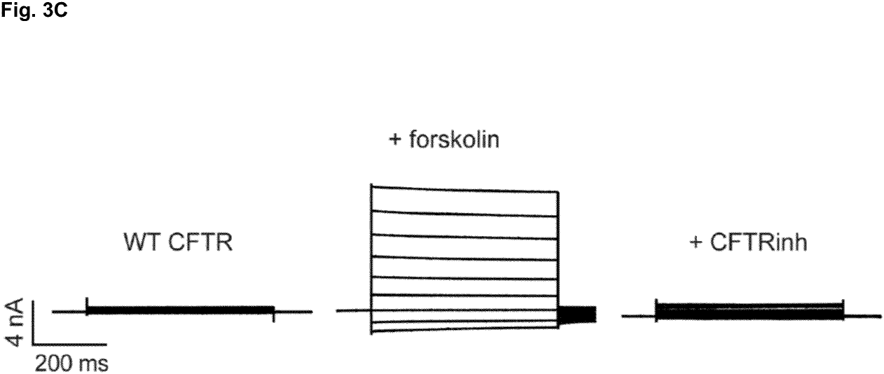

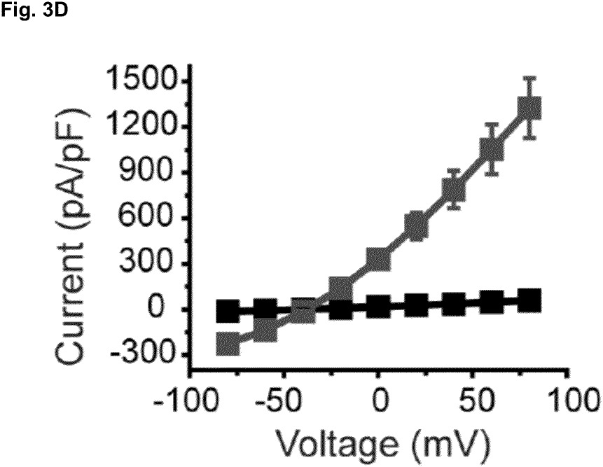

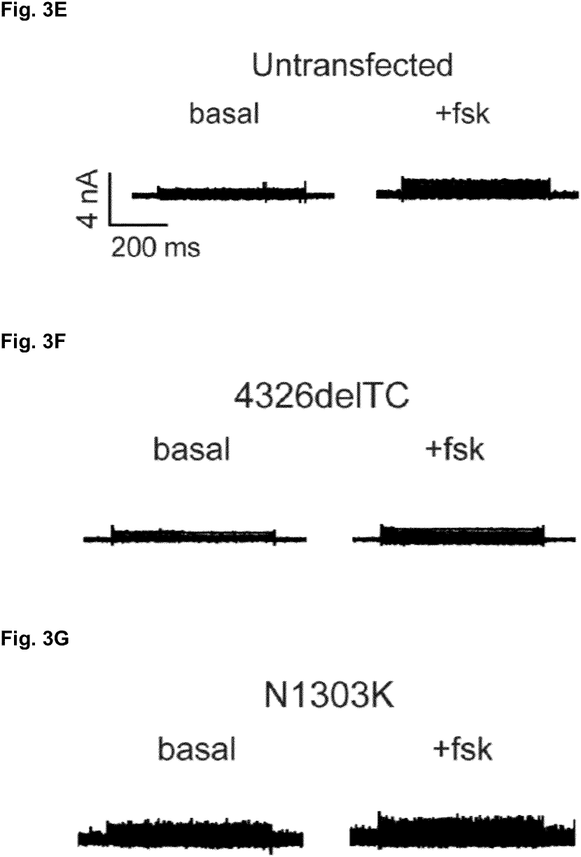

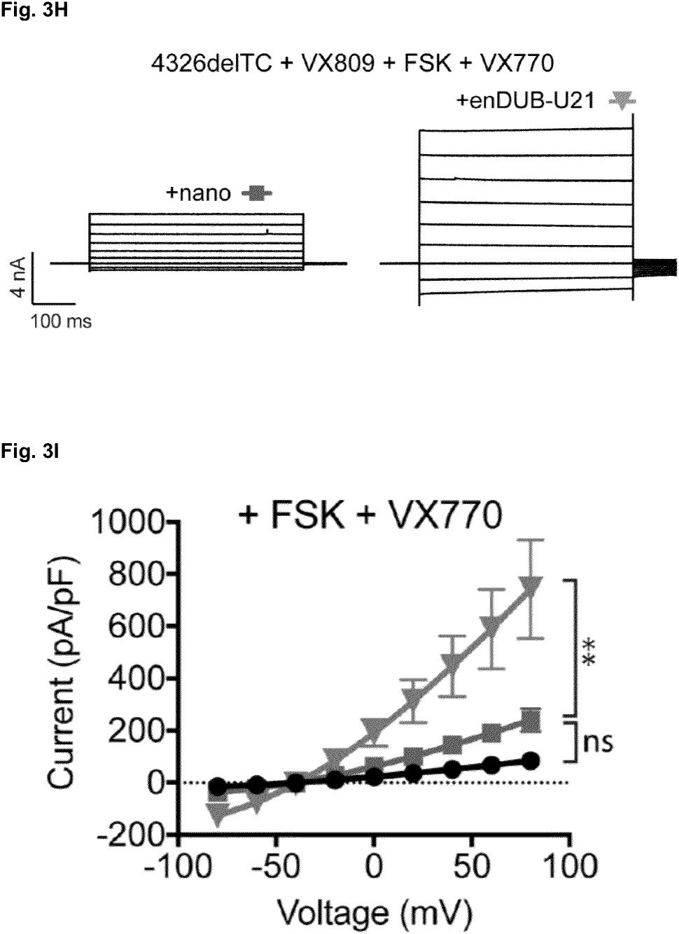

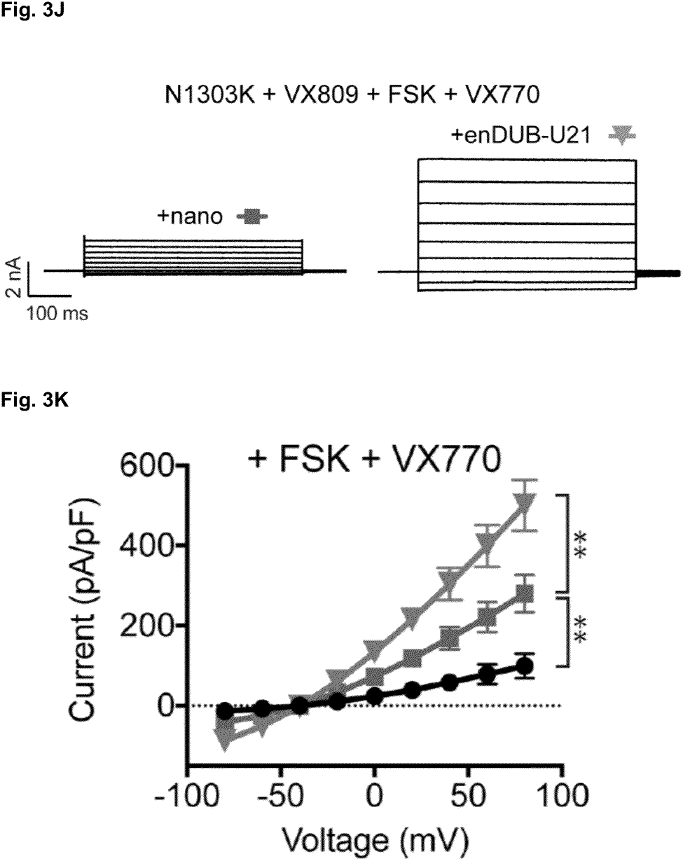

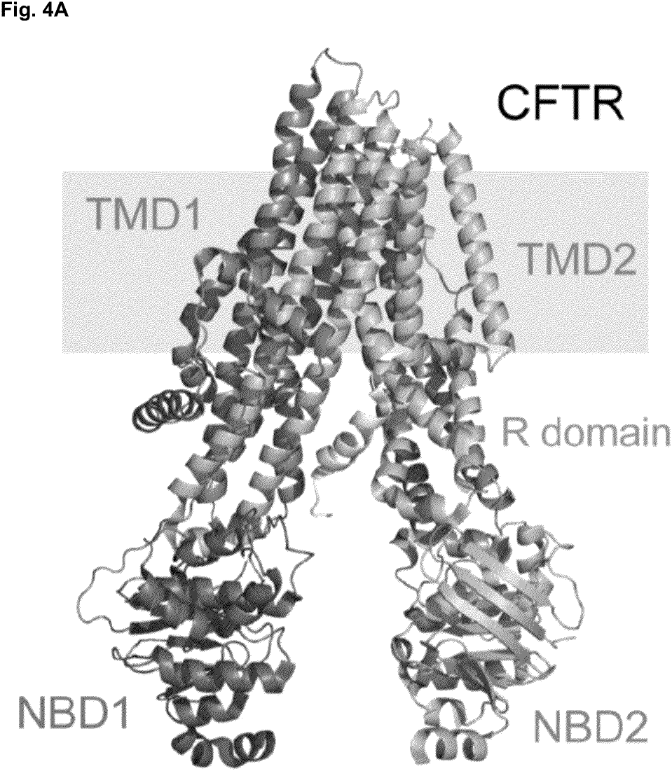

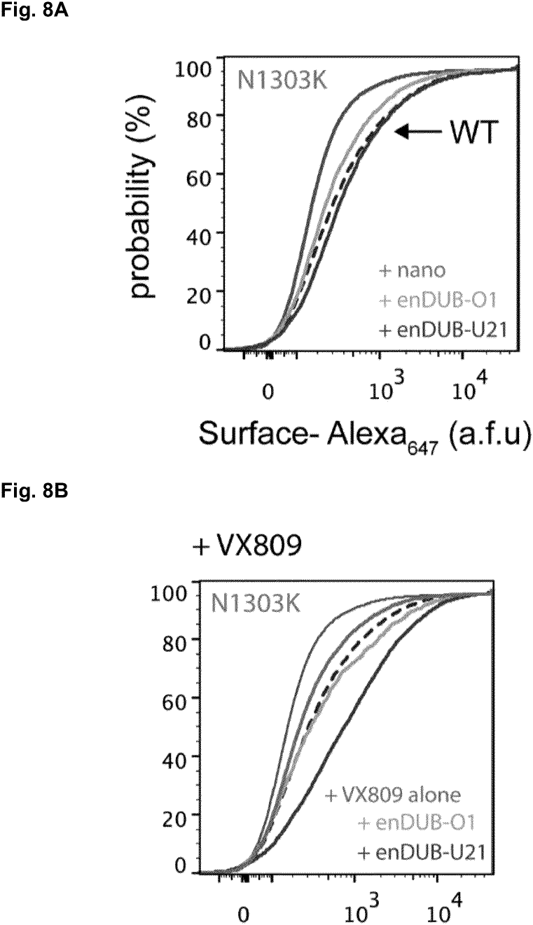

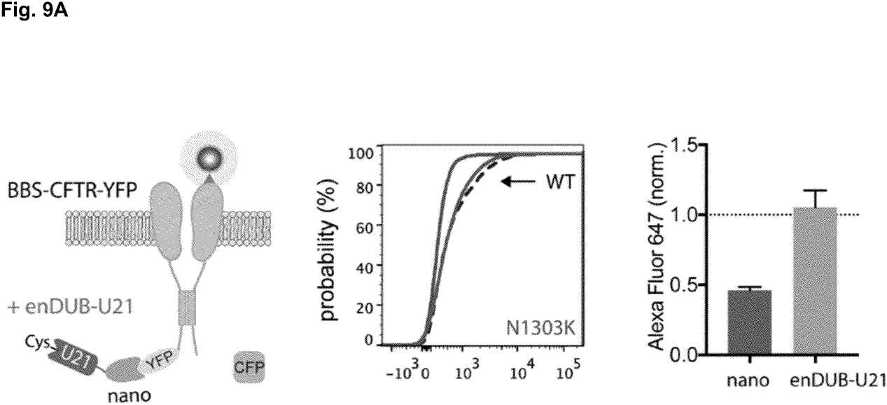

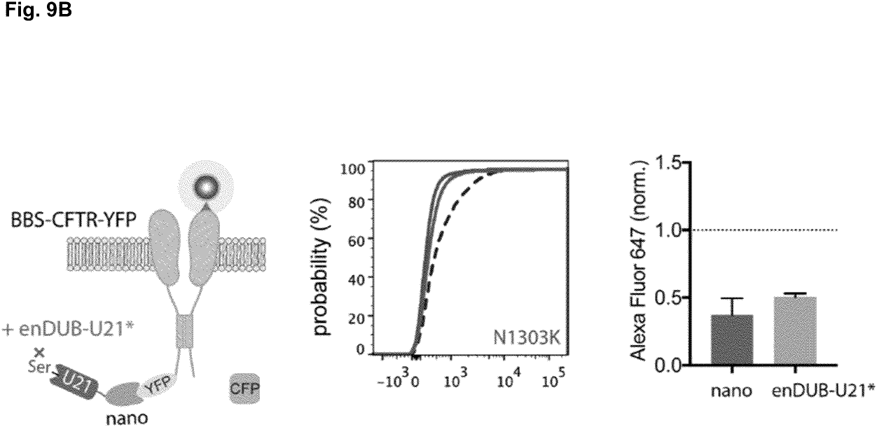

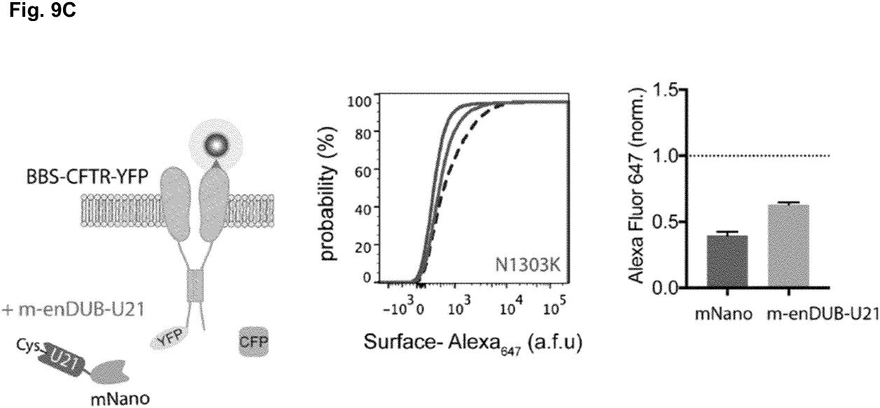

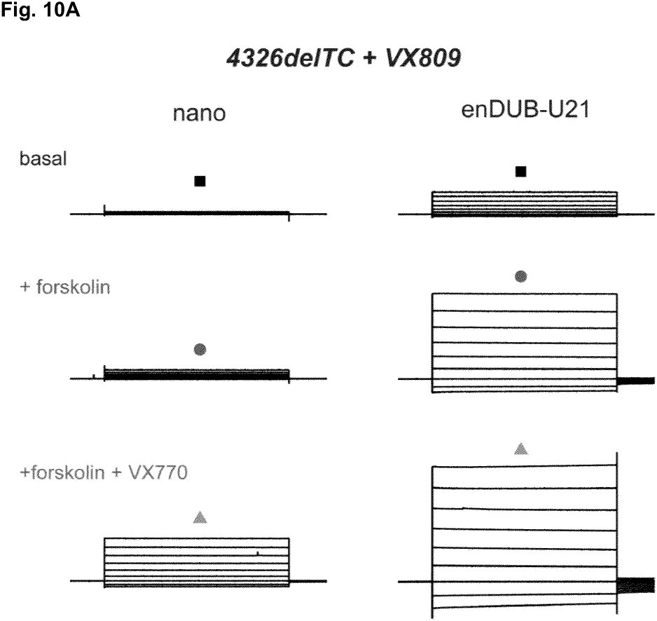

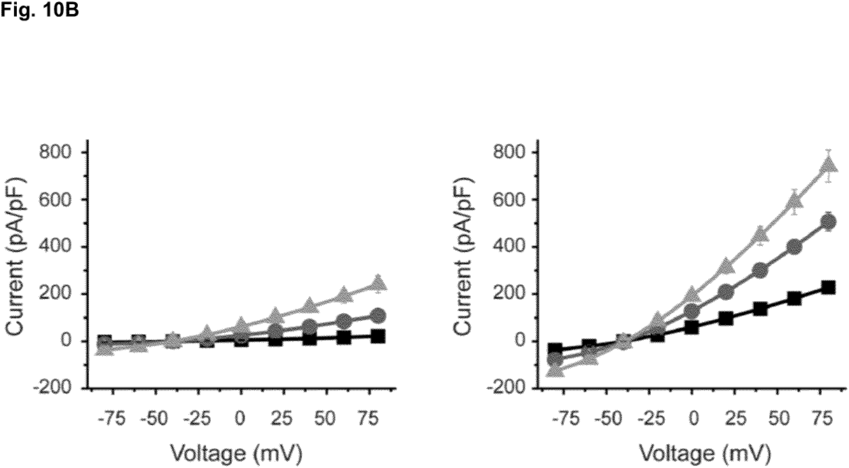

BBS-tagged YFP-CFTR was engineered to enable simultaneous assessment of total channel expression and surface density using flow cytometry, and probed the impact of six distinct mutations previously categorized as Class II (F508del, R560T, N1303K) or Class VI (Q1412X, 4279insA, 4326delTC) mutations, respectively (FIG. 3A). All six mutations markedly impaired channel surface density compared to WT CFTR (FIG. 3B, black bars). Pre-incubation of cells with lumacaftor for 24 hrs did not increase F508del and R560T surface expression, but improved trafficking of the remaining 4 mutations (FIG. 3B, red bars), providing a gold standard corrector benchmark against which to assess the efficacy of enDUBs. We utilized a second enDUB (enDUB-U21) that comprises the catalytic component of ubiquitin-specific protease USP21 (FIG. 3A), which removes all ubiquitin linkage types (Faesen et al. 2011). In pilot experiments, enDUB-U21 was more efficacious for rescuing CFTR trafficking compared to enDUB-O1 (FIGS. 8A-8B), leading us to adopt the former for CFTR experiments. Similar to lumacaftor, enDUB-U21 did not significantly rescue F508del and R560T surface density; however, it was either equal to or more effective in correcting the other four mutations, two of which (N1303K and 4279insA) were rescued to WT CFTR levels (FIG. 3B, blue bars). Both DUB activity and CFTR targeting are required for reversing trafficking deficits as catalytically inactive enDUB-U21 and mCh-targeted enDUB-U21 did not improve surface expression of YFPtagged N1303K (FIGS. 9A-9C). Most importantly, co-applying lumacaftor and enDUB-U21 yielded synergistic rescue of mutant CFTR surface density, suggesting a novel combination corrector therapy for CF (FIG. 3B; green bars).

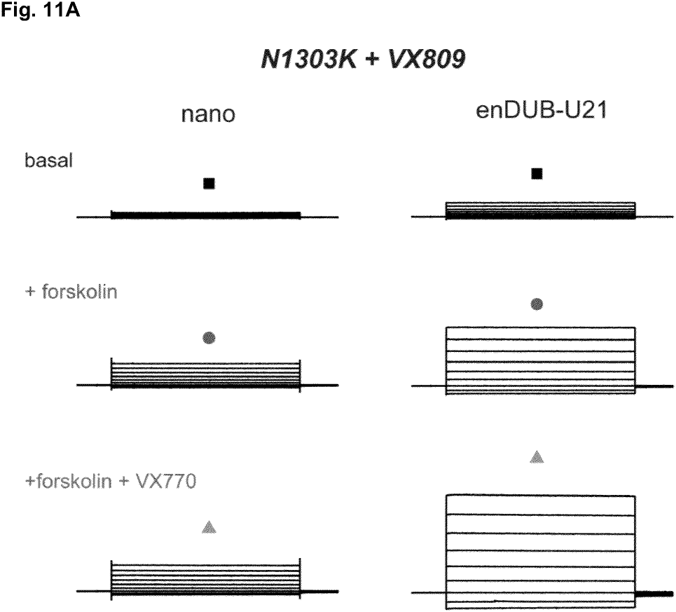

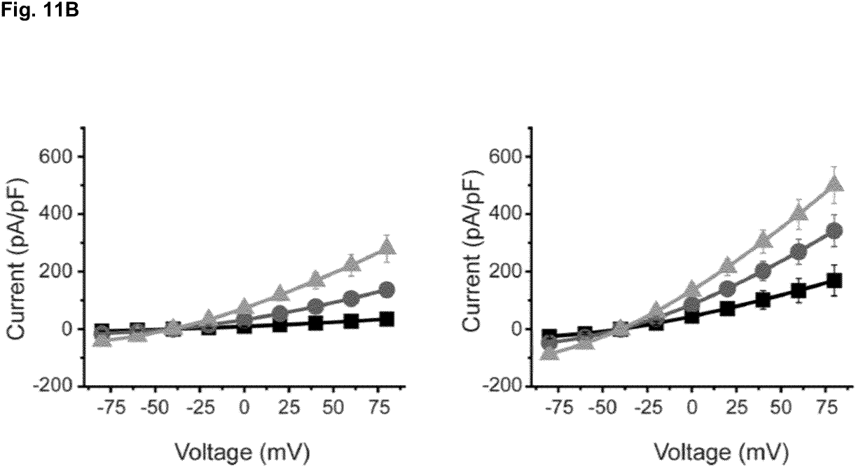

A critical next step was to determine whether enDUB-U21-rescued mutant CFTR channels are functional. We focused on N1303K and 4326delTC to represent Class II and Class VI mutations, respectively. HEK293 cells expressing WT CFTR display robust forskolin-activated chloride currents that are blocked by CFTR inhibitor (FIGS. 3C and 3D), and not observed in untransfected cells (FIG. 3E). By contrast, cells expressing either N1303K or 4326delTC alone yielded no forskolin-induced currents (FIGS. 3F and 3G) consistent with their limited surface trafficking and status as disease-causing mutants. In nano-expressing cells, pre-incubation with lumacaftor yielded relatively small forskolin-induced 4326delTC (FIGS. 10A-10B) and N1303K currents (FIGS. 11A-11B), which were further elevated by ivacaftor (FIGS. 3H-3K). Excitingly, under the same conditions, cells coexpressing enDUB-U21 yielded significantly larger forskolin-stimulated 4326delTC or N1303K currents (FIGS. 10A-10B and FIGS. 11A-11B) which were further enhanced by ivacaftor (FIGS. 3H-3K).

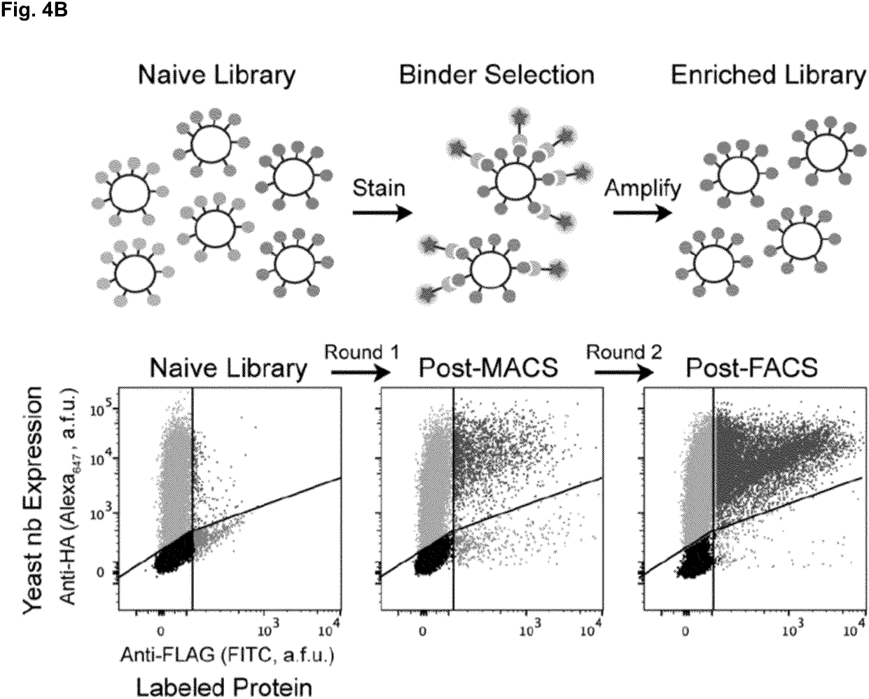

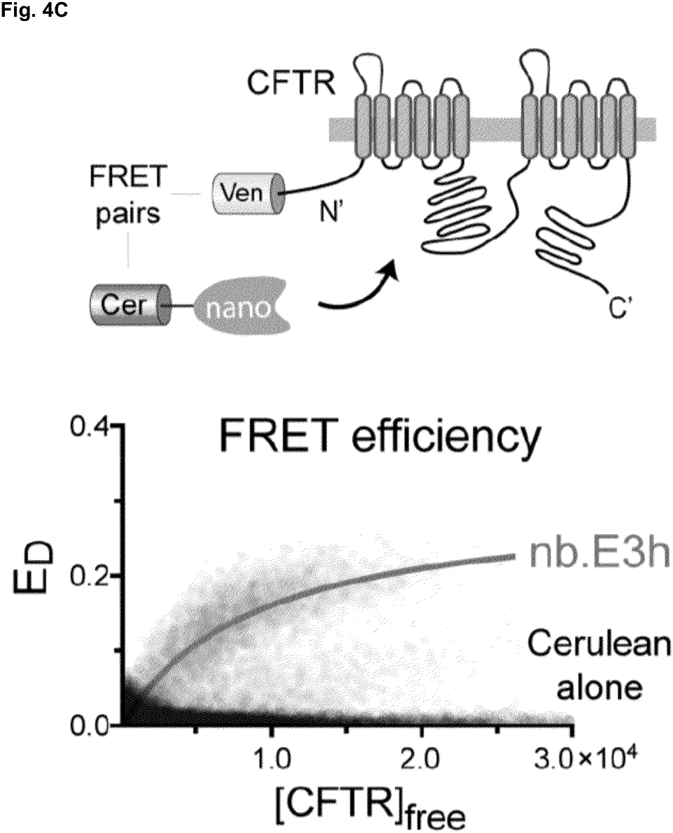

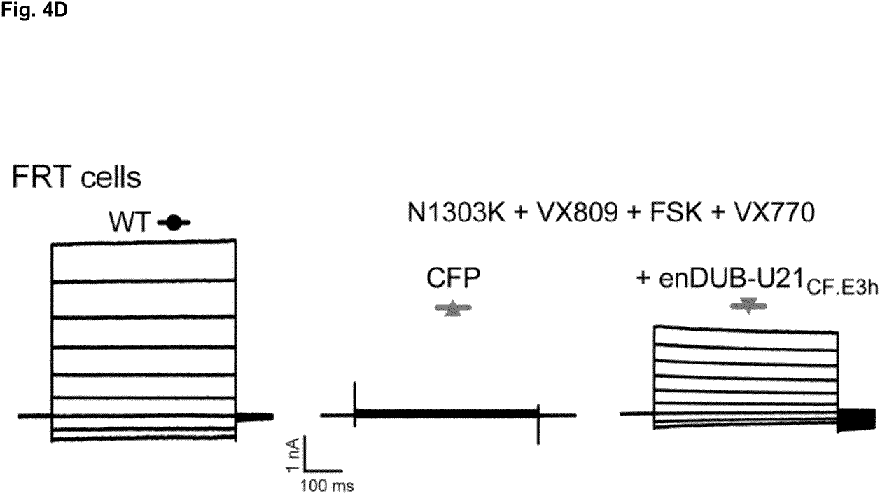

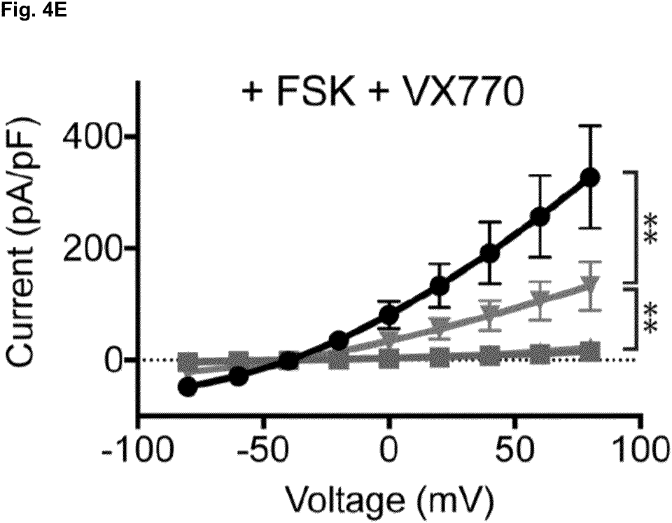

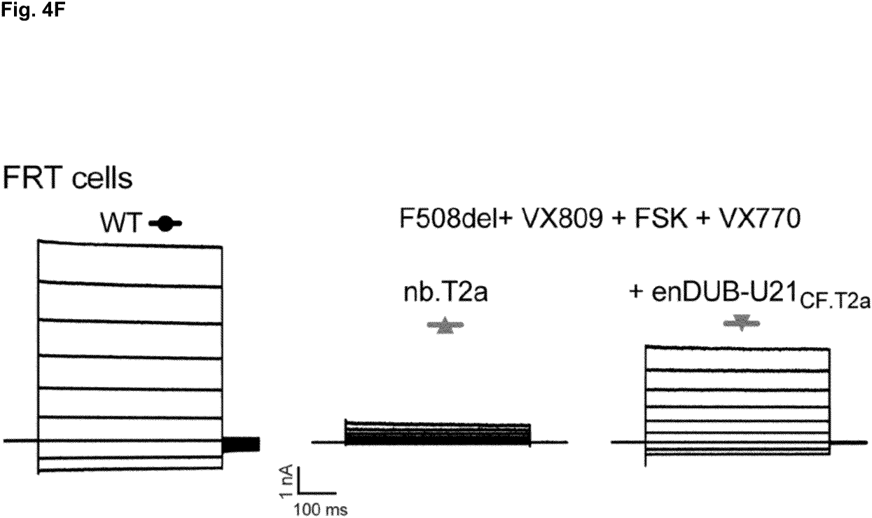

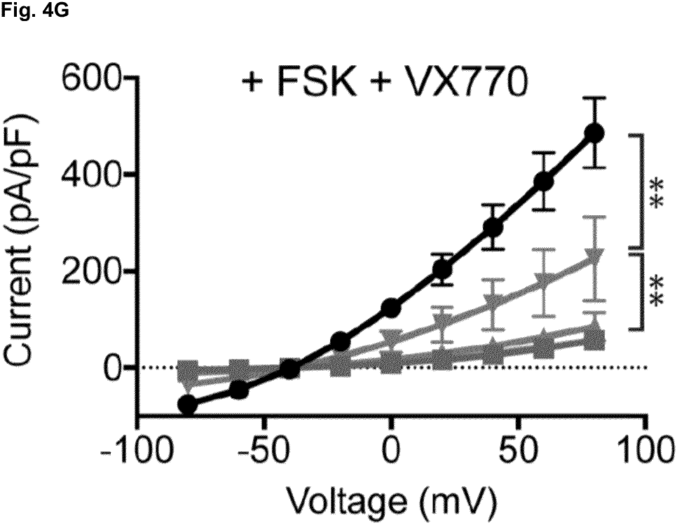

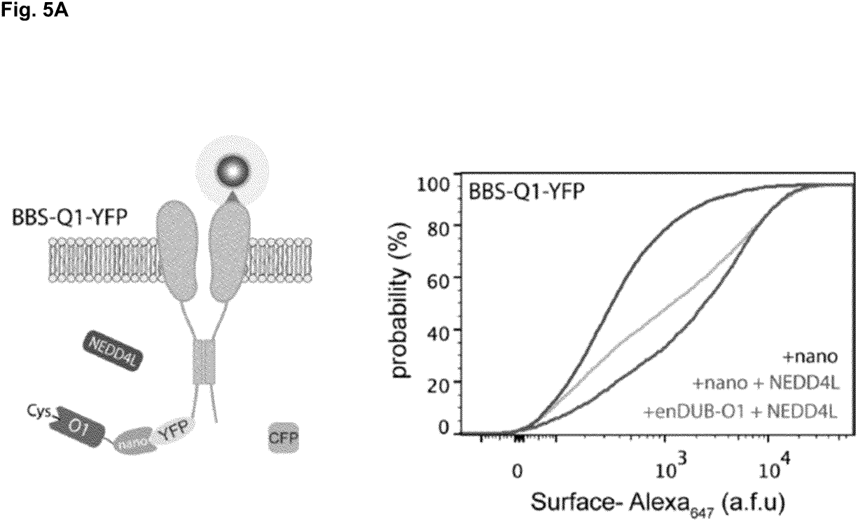

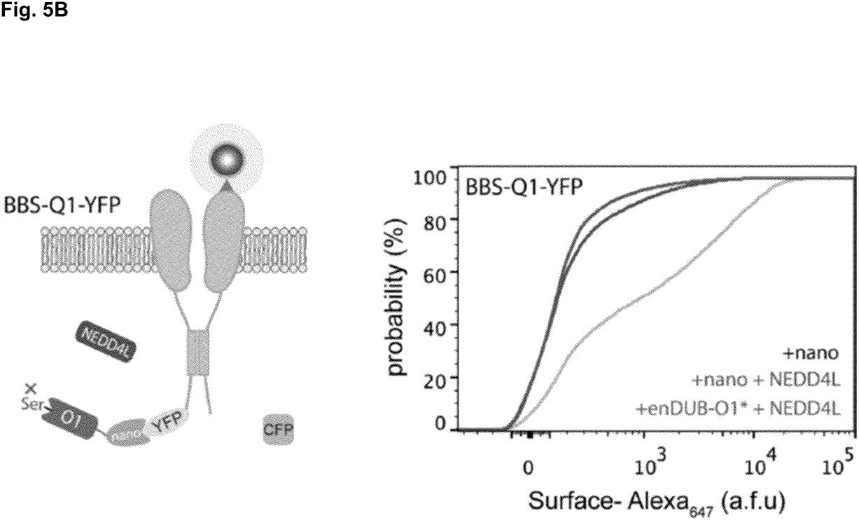

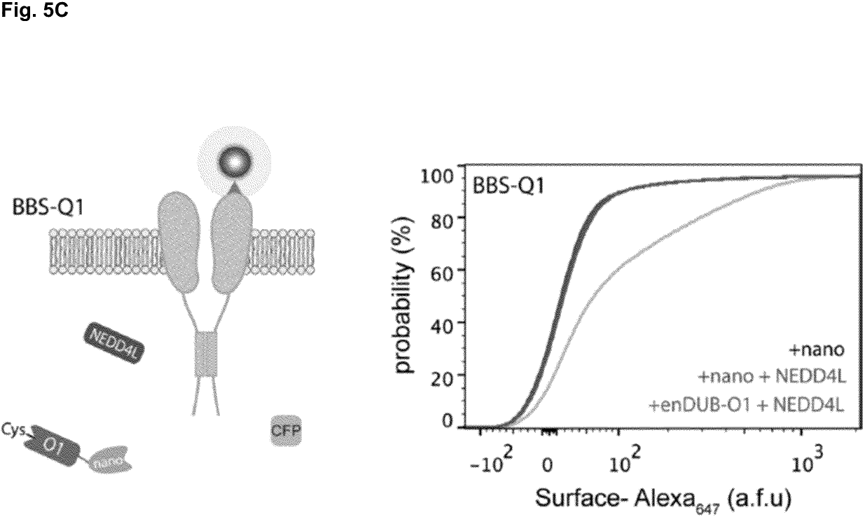

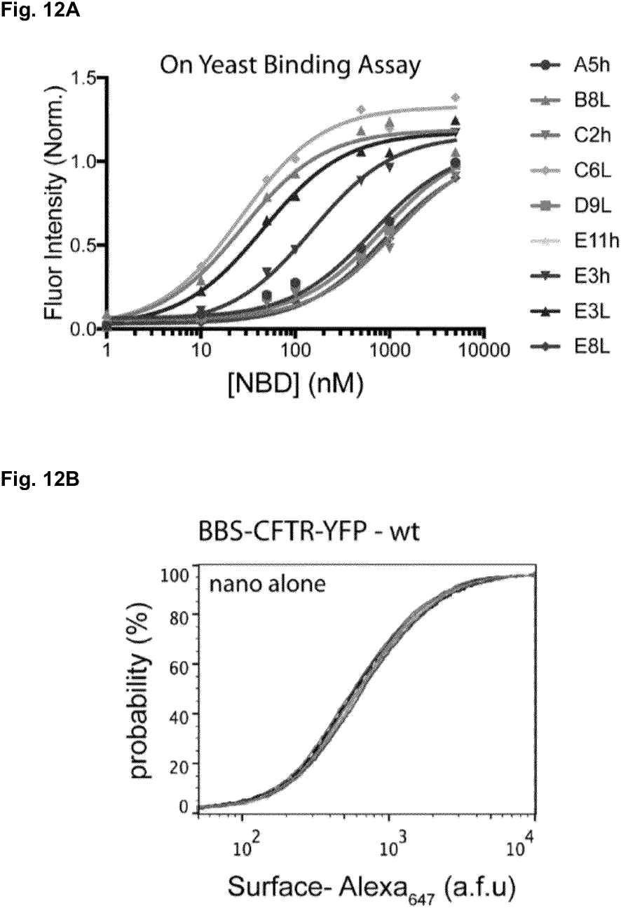

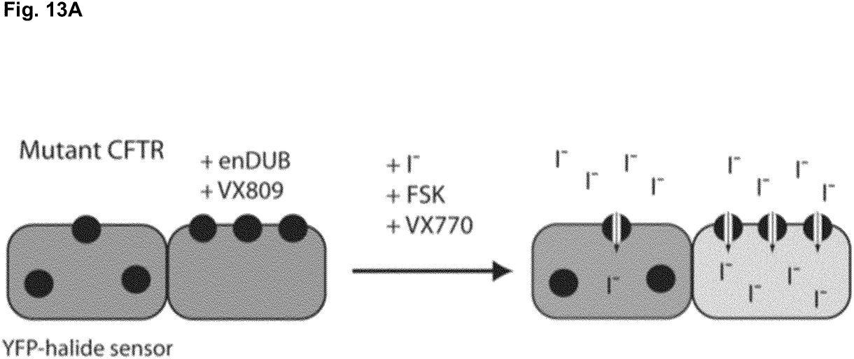

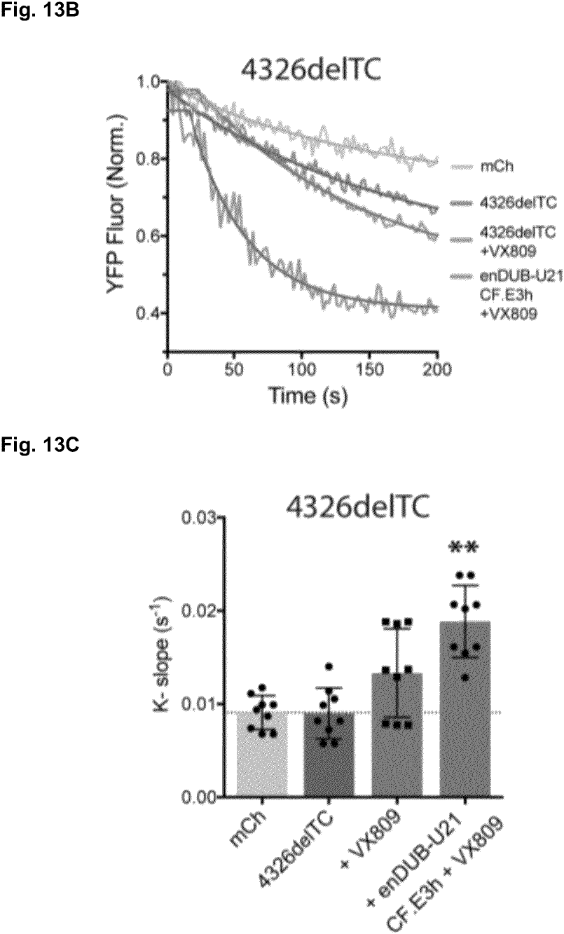

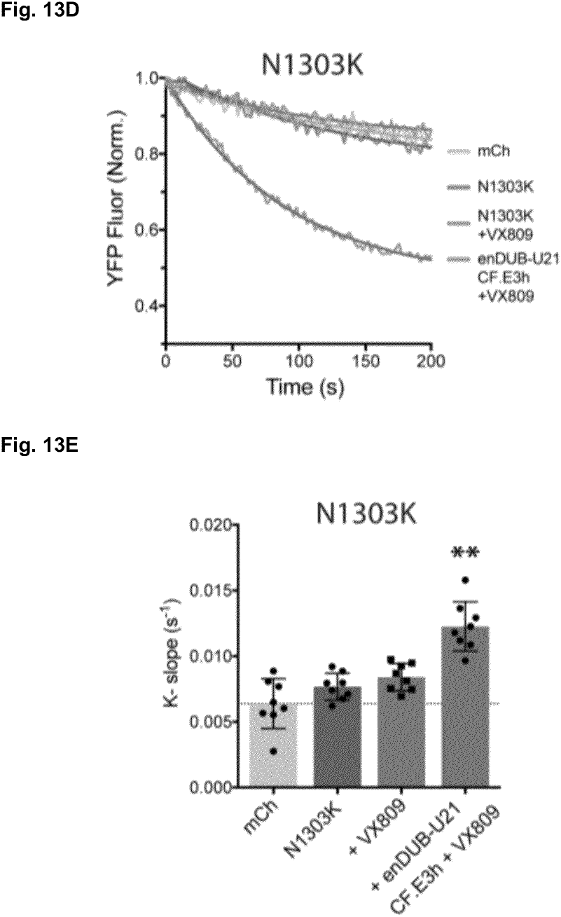

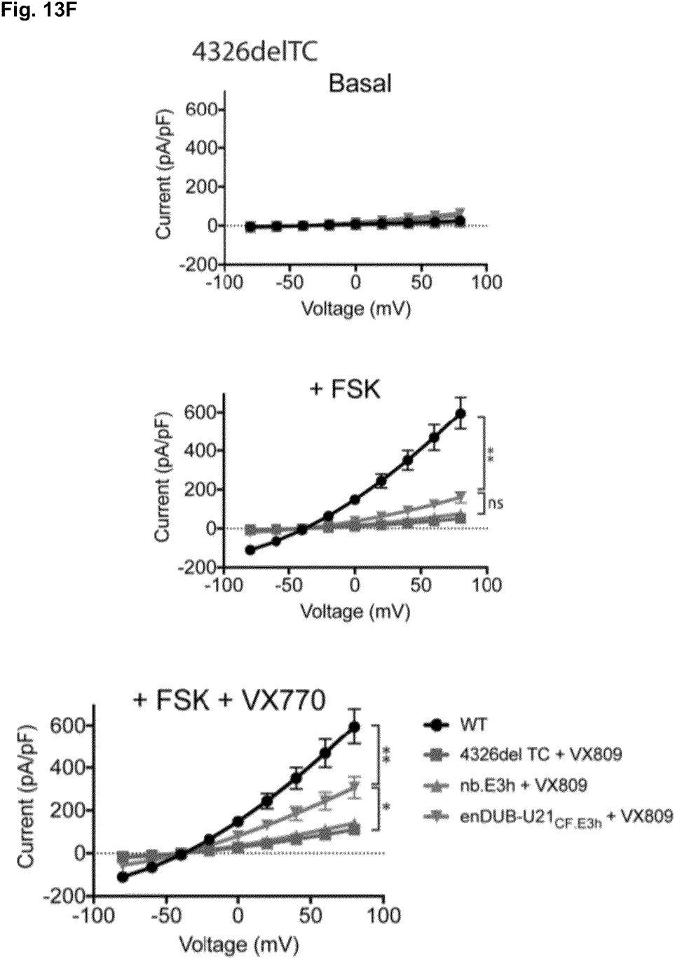

While GFP-targeted enDUBs provided critical proof-of-concept efficacy for the targeted deubiquitination approach in rescuing different trafficking-deficient recombinant mutant channels, a key next step was to develop nanobodies towards CFTR itself to enable targeting of endogenous channels. Accordingly, we used purified CFTR NBD1 domain (FIG. 4A) as bait to identify binders using a yeast nanobody library surface display approach (McMahon et al. 2018) (FIG. 4B). After several rounds of magnetic-activated cell sorting (MACS) and fluorescence-activated cell sorting (FACS) selection we isolated 14 unique nanobody binders with a range of affinities for NBD1 as reported by an on-yeast binding assay (FIG. 12A; Table 1). Reassuringly, when co-expressed with WT CFTR, a number of nanobody binders did not intrinsically interfere with surface trafficking of the channel (FIG. 12B). In pilot studies, we utilized a halide sensitive YFP quenching assay (Galietta et al. 2001) to screen different nanobodies for enDUB-mediated functional rescue of CFTR iodide currents in HEK293 cells (FIG. 13A). We chose one nanobody clone nb.E3h for its superior performance in both halide sensor and patch clamp assays (FIGS. 13A-13F) when converted to an enDUB (termed enDUB-U21_{CF.E3h}). Binding of nb.E3h to full-length CFTR in cells was confirmed by a flow cytometric fluorescence resonance energy transfer (flow-FRET) assay (FIG. 4C).

To test efficacy of the CF-targeted enDUBs in a relevant cellular context, we took advantage of a predictive in vitro CF model-Fischer Rat Thyroid (FRT) epithelial cells stably expressing mutant CFTR channels-that has been used to generate preclinical data preceding clinical trials (Goor et al. 2011; Goor et al. 2009; Yu et al. 2012) and to promote FDA drug label expansion of Kalydeco (ivacaftor) (Ratner, 2017; Durmowicz et al. 2018). Consistent with previous findings (Han et al 2018; Goor et al. 2014), FRT cells stably expressing N1303K channels demonstrated little functional current compared with WT control cells, and were unresponsive to VX809 + VX770 treatment (FIGS. 4D and 4E). Remarkably, enDUB-U21_{CF.E3h} in combination with the same CFTR modulators yielded an impressive rescue of N1303K currents, up to ~40% of WT cells (FIGS. 4D and 4E).

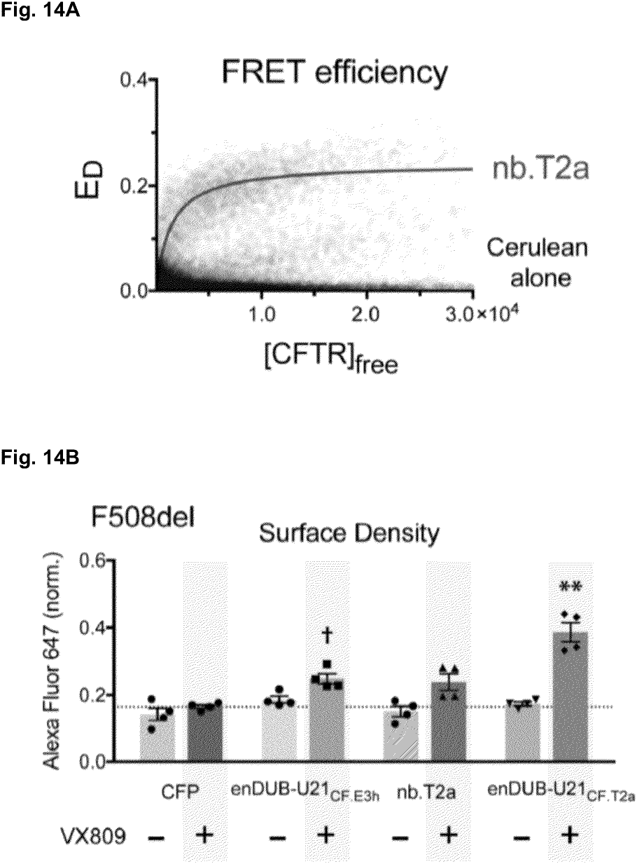

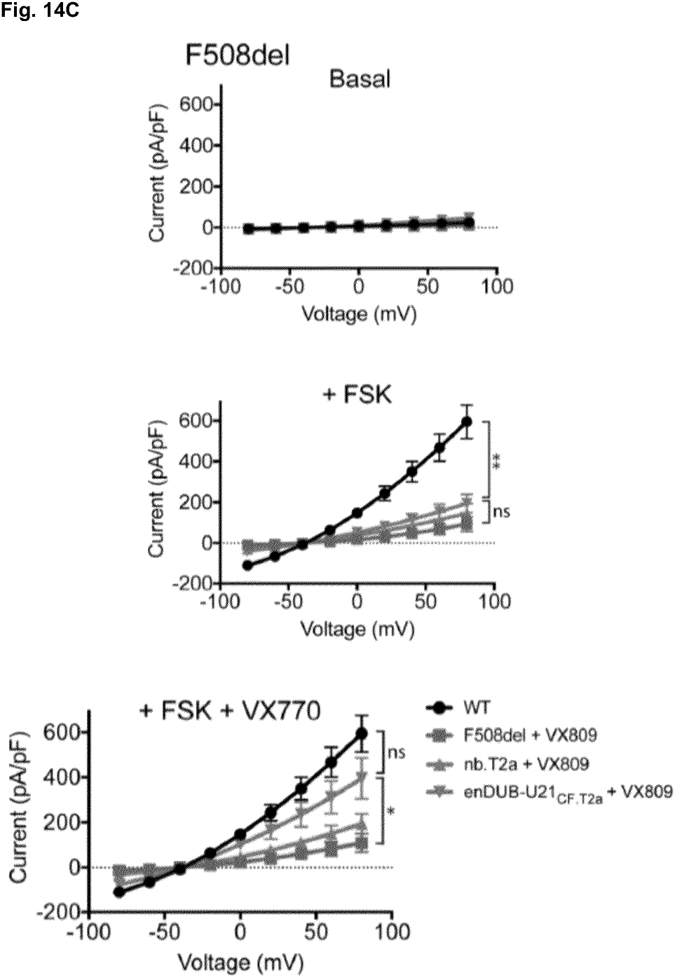

F508del represents the most common CF mutation, with a phenylalanine deletion in NBD1 that leads to deficits in the thermostability of CFTR folding, assembly, and trafficking (Lukacs and Verkman, 2012; Okiyoneda et al. 2013; Okiyoneda et al. 2010). In HEK293 cells, enDUB-U21_{CF.E3h} resulted in only a modest improvement in F508del surface expression in the presence of VX809 (FIGS. 14A-14C). It is hypothesized that an alternate enDUB with a dual capability to; 1) enhance NBD1 thermostability upon binding, and 2) tune CFTR ubiquitin status via catalytic action, would lead to improved F508del rescue. Notably, a recent study developed a nanobody (nb.T2a) that bound isolated wt and F508del NBD1 with thermostabilizing properties in cell free preparations (Sigoillot et al. 2019); however, the functional impact of nb.T2a was not examined on full-length F508del CFTR mutants in situ. We tested the potential synergy of thermostabilizing enDUBs by adapting nb.T2a to our enDUB-U21 system (enDUB-U21_{CF.T2a}). Although nb.T2a expression in combination with VX809 led to a modest increase in F508del surface trafficking, our functionalized enDUB-U21_{CF.T2a} + VX809 demonstrated a significantly enhanced surface rescue in HEK293 cells (FIGS. 14A-14C). Moreover, the superior functional rescue was corroborated in HEK293 patch-clamp studies, with enDUB-U21_{CF.T2a} significantly improving F508del functional currents compared to VX809 ± nb.T2a alone (FIGS. 14A-14C). Finally, in FRT cells stably expressing F508del, combination treatment with enDUB-U21_{CF.T2a} + VX809 + VX770 resulted in a functional F508del rescue to ~45% of WT levels, a substantial improvement compared with VX809 + VX770 ± nb.T2a treatment alone (FIGS. 4F and 4G).

Taken together, our data reveals targeted deubiquitination as a robust strategy to rescue divergent trafficking-impaired ion channels that underlie dissimilar diseases. While highthroughput screening has enabled the identification of pharmacological correctors (such as lumacaftor for CFTR), these typically are only effective on one target channel, and their mechanism of action unknown. On the other hand, low temperature and nonspecific chemical chaperones (e.g. glycerol) (Okiyoneda et al. 2013; Delisle et al. 2004) can rescue distinct subsets of trafficking-impaired ion channels; however, this approach is not amenable to therapeutic development. In this light, enDUBs represent an exciting new mechanism-based strategy, with specificity in targeting and adaptability across different channel types, that can be built upon for custom therapeutic applications. Beyond membrane proteins, it is intriguing to consider opportunities for modulating diverse ubiquitin-dependent processes for distinct protein targets in living cells (Nalepa et al. 2006; Huang and Dixit, 2016). Translating these insights into effective molecular therapies for a variety of diseases is an exciting prospect for future studies.

### Materials and Methods

### Molecular Biology and Cloning of Plasmid Vectors

A customized bicistronic CMV mammalian expression vector (nano-xx-P2A-CFP) was generated as described previously (Kanner et al. 2017); we PCR amplified the coding sequence for GFP nanobody (vhhGFP4) (Rothbauer et al. 2008) and cloned it into xx-P2A-CFP using NheI/AflII sites. To generate the enDUB-O1 construct, we PCR amplified the OTU domain + UIM (residues 287-481) from OTUD1 (Addgene #61405) using AscI/AflII sites separated by a flexible GSG linker. To create the catalytically inactive enDUB-O1*, we introduced a point mutation at the catalytic cysteine residue [C320S] by site-directed mutagenesis. A second custom bicistronic vector (CFP-P2a-nano-xx) was generated as described previously (Kanner et al. 2017). To generate enDUB-U21, we PCR amplified the USP domain (residues 196-565) from USP21 (Addgene #22574) and cloned this fragment into CFP-P2a-nano-xx using AscI/NotI sites. To create the catalytically inactive enDUB-U21*, we introduced a point mutation at the catalytic cysteine residue [C221S] by site-directed mutagenesis. An mChtargeted enDUB-U21 was generated with the mCh nanobody, LaM-4 (Fridy et al. 2014), using a similar cloning strategy as above.

KCNQ1 constructs were made as described previously (Aromolaran et al. 2014). Briefly, overlap extension PCR was used to fuse enhanced yellow fluorescent proteins (EYFP) in frame to the C-terminus of KCNQ1. A 13-residue bungarotoxin-binding site (BBS; TGGCGGTACTACGAGAGCAGCCTGGAGCCCTACCCCGAC; SEQ ID No: 81) (Aromolaran et al. 2014; Sekine-Aizawa and Huganir, 2004) was introduced between residues 148-149 in the extracellular S1-S2 loop of KCNQ1 using the QuikChange Lightning Site-Directed Mutagenesis Kit (Stratagene) according to the manufacturer’s instructions. LQT1 mutations were introduced in the N- and C-termini of KCNQ1 via site-directed mutagenesis. NEDD4L (PCI_NEDD4L; Addgene #27000) was a gift from Joan Massague (Gao et al. 2009).

CFTR constructs were derived from pAd.CB-CFTR (ATCC^{®} 75468). To create CFTRYFP, PCR amplification was used to fuse EYFP to the N-terminus of CFTR. To create BBS-CFTR-YFP, overlap extension PCR was used to introduce the BBS site between residues 901-902 in the fourth extracellular loop (ECL4) of CFTR (Peters et al. 2011). CF patient-specific mutations were introduced in NBD1, NBD2, and C-terminus of CFTR via site-directed mutagenesis. YFP halide sensor (EYFP H148Q/I152L) was used (Galietta et al. 2001) (Addgene #25872). To generate CF-targeted enDUBs, we created a modular CFP-P2axx-U21 vector with an extended (GGGGS)x5(GGGTG) linker upstream the USP domain. Select NBD1 nanobody binders were then cloned using BglII/AscI sites.

### Generation of Adenoviral Vectors

Adenoviral vectors were generated using the pAdEasy system (Stratagene) according to manufacturer’s instructions as previously described (Aromolaran et al. 2014). Plasmid shuttle vectors (pShuttle CMV) containing cDNA for nano-P2A-CFP, WT KCNQ1-YFP, and G589D KCNQ1-YFP were linearized with Pmel and electroporated into BJ5183-AD-1 electrocompetent cells pre-transformed with the pAdEasy-1 viral plasmid (Stratagene). PacI restriction digestion was used to identify transformants with successful recombination. Positive recombinants were amplified using XL-10-Gold bacteria, and the recombinant adenoviral plasmid DNA linearized with PacI digestion. HEK cells were cultured in 60 mm diameter dishes at 70-80% confluency and transfected with PacI-digested linearized adenoviral DNA. Transfected plates were monitored for cytopathic effects (CPEs) and adenoviral plaques. Cells were harvested and subjected to three consecutive freeze-thaw cycles, followed by centrifugation (2,500 × g) to remove cellular debris. The supernatant (2 mL) was used to infect a 10 cm dish of 90% confluent HEK293 cells. Following observation of CPEs after 2-3 days, cell supernatants were used to re-infect a new plate of HEK293 cells. Viral expansion and purification was carried out as previously described (Aromolaran et al. 2014). Briefly, confluent HEK293 cells grown on 15 cm culture dishes (x8) were infected with viral supernatant (1 mL) obtained as described above. After 48 hours, cells from all of the plates were harvested, pelleted by centrifugation, and resuspended in 8 mL of buffer containing (in mM) Tris·HCl 20, CaCl₂ 1, and MgCl₂ 1 (pH 8.0). Cells were lysed by four consecutive freeze-thaw cycles and cellular debris pelleted by centrifugation. The virus-laden supernatant was purified on a cesium chloride (CsCl) discontinuous gradient by layering three densities of CsCl (1.25, 1.33, and 1.45 g/mL). After centrifugation (50,000 rpm; SW41Ti Rotor, Beckman-Coulter Optima L-100K ultracentrifuge; 1 h, 4° C.), a band of virus at the interface between the 1.33 and 1.45 g/mL layers was removed and dialyzed against PBS (12 h, 4° C.). Adenoviral vector aliquots were frozen in 10% glycerol at -80° C. until use. Generation of enDUB-O1-P2A-CFP was performed by Vector Biolabs (Malvern, PA).

### Cell Culture and Transfections

Human embryonic kidney (HEK293) cells were used. Cells were mycoplasma free, as determined by the MycoFluor Mycoplasma Detection Kit (Invitrogen). Low passage HEK293 cells were cultured at 37° C. in DMEM supplemented with 8% fetal bovine serum (FBS) and 100 mg/mL of penicillin-streptomycin. HEK293 cell transfection was accomplished using the calcium phosphate precipitation method. Briefly, plasmid DNA was mixed with 62 µL of 2.5 M CaCl₂ and sterile deionized water (to a final volume of 500 µL). The mixture was added dropwise, with constant tapping to 500 µL of 2× Hepes buffered saline containing (in mM): Hepes 50, NaCl 280, Na₂HPO₄ 1.5, pH 7.09. The resulting DNA-calcium phosphate mixture was incubated for 20 min at room temperature and then added dropwise to HEK293 cells (60 - 80% confluent). Cells were washed with Ca²⁺-free phosphate buffered saline after 4-6 h and maintained in supplemented DMEM.

Chinese hamster ovary (CHO) cells were obtained from ATCC and cultured at 37° C. in Kaighn’s Modified Ham’s F-12K (ATCC) supplemented with 8% FBS and 100 mg/mL of penicillin-streptomycin. CHO cells were transiently transfected with desired constructs in 35 mm tissue culture dishes—KCNQ1 (0.5 µg) and nano-P2A-CFP (0.5 µg) or enDUBO1-P2A-CFP (0.5 µg) using X-tremeGENE HP (1:2 DNA/reagent ratio) according to the manufacturers’ instructions (Roche).

FRT epithelial cells stably-expressing WT and mutant CFTR channels (Han et al. 2018) were used. FRT cells were maintained at 37° C. in Ham’s F-12 Coon’s modification (Sigma) supplemented with 5% FBS, 100 mg/mL of penicillin-streptomycin, 7.5% w/v sodium bicarbonate, and 100 µg/mL Hygromycin (Invitrogen). FRT cell transient transfection was accomplished using Lipofectamine 3000 according to the manufacturer’s instructions (Thermo).

Isolation of adult guinea pig cardiomyocytes was performed in accordance with the guidelines of Columbia University Animal Care and Use Committee. Prior to isolation, plating dishes were pre-coated with 15 µg/mL laminin (Gibco). Adult Hartley guinea pigs (Charles River) were euthanized with 5% isoflurane, hearts were excised and ventricular myocytes isolated by first perfusing in KH solution (mM): 118 NaCl, 4.8 KCl, 1 CaCl₂ 25 HEPES, 1.25 K₂HPO₄, 1.25 MgSO₄, 11 glucose, 0.02 EGTA, pH 7.4, followed by KH solution without calcium using a Langendorff perfusion apparatus. Enzymatic digestion with 0.3 mg/mL Collagenase Type 4 (Worthington) with 0.08 mg/mL protease and 0.05% BSA was performed in KH buffer without calcium for six minutes. After digestion, 40 mL of a high K⁺ solution was perfused through the heart (mM): 120 potassium glutamate, 25 KCl, 10 HEPES, 1 MgCl₂, and 0.02 EGTA, pH 7.4. Cells were subsequently dispersed in high K⁺ solution. Healthy rod-shaped myocytes were cultured in Medium 199 (Life Technologies) supplemented with (mM): 10 HEPES (Gibco), 1× MEM non-essential amino acids (Gibco), 2 L-glutamine (Gibco), 20 D-glucose (Sigma Aldrich), 1% vol/vol penicillin-streptomycin-glutamine (Fisher Scientific), 0.02 mg/mL Vitamin B-12 (Sigma Aldrich) and 5% (vol/vol) FBS (Life Technologies) to promote attachment to dishes. After 5 hrs, the culture medium was switched to Medium 199 with 1% (vol/vol) serum, but otherwise supplemented as described above. Cultures were maintained in humidified incubators at 37° C. and 5% CO₂.

### Flow Cytometry Assay of Total and Surface Channels

Cell surface and total ion channel pools were assayed by flow cytometry in live, transfected HEK293 cells as previously described (Kanner et al. 2017; Kanner et al. 2018). Briefly, 48 hrs post-transfection, cells cultured in 12-well plates gently washed with ice cold PBS containing Ca²⁺ and Mg²⁺ (in mM: 0.9 CaCl₂, 0.49 MgCl₂, pH 7.4), and then incubated for 30 min in blocking medium (DMEM with 3% BSA) at 4° C. HEK293 cells were then incubated with 1 µM Alexa Fluor 647 conjugated α-bungarotoxin (BTX₆₄₇; Life Technologies) in DMEM/3% BSA on a rocker at 4° C. for 1 hr, followed by washing three times with PBS (containing Ca²⁺ and Mg²⁺ ). Cells were gently harvested in Ca²⁺-free PBS, and assayed by flow cytometry using a BD LSRII Cell Analyzer (BD Biosciences, San Jose, CA, USA). CFP- and YFPtagged proteins were excited at 405 and 488 nm, respectively, and Alexa Fluor 647 was excited at 633 nm.

### FRET Flow Cytometric Assay

FRET binding assays were performed via flow cytometry in live, transfected HEK293 cells as previously described (Lee et al. 2016). Briefly, cells were cultured for 24 hours post-transfection and incubated for 2-4 hrs with cycloheximide (100 µM) and 30 min with H89 (30 µM) prior to analysis to reduce cell variation in fluorescent protein maturity and basal kinase activity. Cells were gently washed with ice cold PBS (containing Ca²⁺ and Mg²⁺ ), harvested in Ca²⁺-free PBS, and assayed by flow cytometry using a BD LSRII Cell Analyzer (BD Biosciences, San Jose, CA, USA). Cerulean (Cer), Venus (Ven), and FRET signals were analyzed using the following laser / filter set configurations: BV421 (Ex: 405 nm, Em: 450/50), FITC (Ex: 488 nm, Em: 525/50), and BV520 (Ex: 405 nm, Em: 525/50), respectively. Several controls were prepared for each experiment, including untransfected blanks for background subtraction, single color Ven and Cer for spectral unmixing, Cer + Ven co-expressed together for concentration-dependent spurious FRET estimation, as well as a series of Cer-Ven dimers for FRET calibration. Custom Matlab software was used to analyze FRET donor / acceptor efficiency and generate FRET binding curves as a function of [acceptor]_{free} and [donor]_{free}.

### Electrophysiology

For potassium channel measurements, whole-cell membrane currents were recorded at room temperature in CHO cells using an EPC-10 patch-clamp amplifier (HEKA Electronics) controlled by the PatchMaster software (HEKA). A coverslip with adherent CHO cells was placed on the glass bottom of a recording chamber (0.7-1 mL in volume) mounted on the stage of an inverted Nikon Eclipse Ti-U microscope. Micropipettes were fashioned from 1.5 mm thin-walled glass and fire-polished. Internal solution contained (mM): 133 KCl, 0.4 GTP, 10 EGTA, 1 MgSO₄, 5 K₂ATP, 0.5 CaCl₂, and 10 HEPES (pH 7.2). External solution contained (in mM): 147 NaCl, 4 KCl, 2 CaCl2, and 10 HEPES (pH 7.4). Pipette resistance was typically 1.5 MΩ when filled with internal solution. I-V curves were generated from a family of step depolarizations (-40 to +100 mV in 10 mV steps from a holding potential of -80 mV). Currents were sampled at 20 kHz and filtered at 5 kHz. Traces were acquired at a repetition interval of 10 s.

Whole-cell recordings of cardiomyocytes were performed 48-72 hrs after infection. Internal and external solutions were used as above. Slow voltage ramp protocol (from -80 mv to +100 mV over 2 s) was used to evoke whole-cell currents. Action potential recordings under current clamp were obtained via 0.25 Hz stimulation with short current pulses (150 pA. 10 ms).

For CFTR channel measurements, whole-cell recordings were carried out in HEK293 and FRT cells at room temperature. Internal solution contained (mM): 113 L-aspartic acid, 113 CsOH, 27 CsCl, 1 NaCl, 1 MgCl₂, 1 EGTA, 10 TES, 3 MgATP (pH 7.2). External contained (in mM): 145 NaCl, 4 CsCl, 1 CaCl₂, 1 MgCl₂, 10 glucose, and 10 TES (pH 7.4). I-V curves were generated from a family of step depolarizations (-80 to +80 mV in 20 mV steps from a holding potential of -40 mV). CFTR currents were activated by perfusion with 10 µM forskolin. In experiments utilizing lumacaftor (3 µM), the drug was added for 24 hrs posttransfection and incubated at 37° C. Ivacaftor was used acutely at 5 µM concentration. Currents were sampled at 20 kHz and filtered at 7 kHz. Traces were acquired at a repetition interval of 10 s.

### Immunoprecipitation and Western Blotting

HEK293 cells were washed once with PBS without Ca²⁺, harvested, and resuspended in RIPA lysis buffer containing (in mM) Tris (20, pH 7.4), EDTA (1), NaCl (150), 0.1% (wt/vol) SDS, 1% Triton X-100, 1% sodium deoxycholate and supplemented with protease inhibitor mixture (10 µL/ mL, Sigma-Aldrich), PMSF (1 mM, Sigma-Aldrich), Nethylmaleimide (2 mM, Sigma-Aldrich) and PR-619 deubiquitinase inhibitor (50 µM, LifeSensors). Lysates were prepared by incubation at 4° C. for 1 hr, with occasional vortex, and cleared by centrifugation (10,000 × g, 10 min, 4° C.). Supernatants were transferred to new tubes, with aliquots removed for quantification of total protein concentration determined by the bis-cinchonic acid protein estimation kit (Pierce Technologies). Lysates were pre-cleared by incubation with 10 µL Protein A/G Sepharose beads (Rockland) for 40 min at 4° C. and then incubated with 0.75 µg anti-Q1 (Alomone) for 1 hr at 4° C. Equivalent total protein amounts were added to spin-columns containing 25 µL Protein A/G Sepharose beads, tumbling overnight at 4° C. Immunoprecipitates were washed 3 times with RIPA buffer, twice with RIPA-500 mM NaCl, spun down at 500 × g, eluted with 40µL of warmed sample buffer [50 mM Tris, 10% (vol/vol) glycerol, 2% SDS, 100 mM DTT, and 0.2 mg/mL bromophenol blue], and boiled (55° C., 15 min). Proteins were resolved on a 4-12% Bis Tris gradient precast gel (Life Technologies) in Mops-SDS running buffer (Life Technologies) at 200 V constant for ~1 h. We loaded 10 µL of the PageRuler Plus Prestained Protein Ladder (10-250 kDa, Thermo Fisher) alongside the samples. Protein bands were transferred by tank transfer onto a nitrocellulose membrane in transfer buffer (25 mM Tris pH 8.3, 192 mM glycine, 15% (vol/vol) methanol, and 0.1% SDS). The membranes were blocked with a solution of 5% nonfat milk (BioRad) in trisbuffered saline-tween (TBS-T) (25 mM Tris pH 7.4, 150 mM NaCl, and 0.1% Tween-20) for 1 hr at RT and then incubated overnight at 4° C. with primary antibodies (anti-Q1, Alomone) in blocking solution. The blots were washed with TBS-T three times for 10 min each and then incubated with secondary horseradish peroxidase-conjugated antibody for 1 hr at RT. After washing in TBS-T, the blots were developed with a chemiluminiscent detection kit (Pierce Technologies) and then visualized on a gel imager. Membranes were then stripped with harsh stripping buffer (2% SDS, 62 mM Tris pH 6.8, 0.8% ß-mercaptoethanol) at 50° C. for 30 min, rinsed under running water for 2 min, and washed with TBST (3×, 10 min). Membranes were pre-treated with 0.5% glutaraldehyde and reblotted with anti-ubiquitin (VU1, LifeSensors) as per the manufacturers’ instructions.

### Yeast Surface Display for Nanobody Binders

Isolation of nanobody binders were performed using a yeast surface display library approach previously described (McMahon et al. 2018). Human NBD1 (residues 387-646, Δ405-436) construct with an N-terminal Hisx6-Smt3 fusion was obtained from Arizona State University Plasmid Repository (clone: HsCD00287374). A FLAG tag was inserted immediately downstream the Hisx6-Smt3 tag using Gibson assembly. Proteins were expressed and His-purified via custom order (Genscript). The Hisx6-Smt3 tag was removed using SUMO protease kit (Invitrogen), with Ulp1 protease incubation overnight at 4° C. and subsequent affinity chromatography purification (HisPur spin columns; Thermo). A naïve yeast library (6 × 10⁹ yeast) was incubated at 25° C. in galactose-containing tryptophan drop-out (Trp-) media for 2-3 days to induce nanobody expression. Induced cells were washed and resuspended in selection buffer (PBS, 0.1% BSA, 5 mM maltose). First round of magneticactivated cell sorting (MACS) selection began with a preclearing step, incubating yeast with anti-FLAG M2-FITC conjugated antibodies (Sigma) and anti-FITC microbeads (Miltenyi) for 30 min at 4° C. and passing them through an LD column (Miltenyi) to remove antibody/microbead binders. NBD1-binding nanobodies were then MACS-enriched by incubating precleared yeast with 500 nM Hisx6-Smt3-FLAG-NBD1 and anti-FLAG M2-FITC for 1 hr at 4° C., followed by a wash in selection buffer, and incubation with anti-FITC microbeads for 20 min at 4° C. Labeled yeast were passed through an LS column (Miltenyi), washed three times with selection buffer, and eluted by removing the MACS magnetic stand (Miltenyi). Enriched NBD1 binders were grown up in glucose-containing Trp- media overnight at 30° C. Induction of nanobody expression was repeated with enriched NBD1 libraries (~1 × 10⁸ yeast) by incubation in galactose Trp- media as outlined above. Subsequently, two rounds of positive selection were performed via fluorescenceactivated cell sorting (FACS), first by incubating induced cells (~5 × 10⁶ yeast here, and thereafter) with 500 nM Hisx6-Smt3-FLAG-NBD1, and next with 500 nM FLAG-NBD1 to remove any Smt3 binders. Nonspecific FITC conjugated antibody binders were removed with a third round, negative selection FACS, incubating cells with anti-FLAG FITC alone. Finally, high affinity NBD1 binders were selected by incubation with 100 nM FLAG-NBD1, FACS sorted as single cells into 96-well plates, and grown up as monoclonal colonies for binding validation studies and plasmid isolation. Unique, validated NBD1 binders were subjected to on-yeast Kd measurements by labeling cells (~10⁵ yeast) with serial dilutions of FLAG-NBD1 (in nM): 5000, 1000, 500, 100, 50, 10, and 1.

### YFP Halide Quenching Assay

A YFP halide quenching plate-reader assay was adapted from previous work (Galietta et al. 2001). Briefly, HEK293 cells were split onto 24-well black-wall plates (VisiPlate; PerkinElmer) and cotransfected with halide-sensitive eYFP (H148Q/I152L), mCh alone or mCh-tagged mutant CFTR channels, and CFP alone or CFP-P2a CF-targeted enDUB-U21 constructs. After 2-3 days, cells were washed once with PBS (containing Ca²⁺ and Mg²⁺) and incubated at 37° C. for 30 min in 200 µL PBS (containing 145 mM NaCl). Baseline YFP readings (Ex: 510 nm, Em: 538 nm) were taken using a SpectraMax M5 plate-reader (Molecular Devices). An equal amount of 2× activation solution, containing iodide, was added to obtain a final concentration (70 mM Nal, 10 µM forskolin, 5 µM VX770), and a time series recording YFP fluorescence was taken every 2 s. Assays were performed at 37° C.

### Confocal Microscopy

Cells were plated onto 35 mm MatTek dishes (MatTek Corporation). Cardiomyocytes were fixed with 4% formaldehyde for 10 min at room temperature (RT). Live HEK293 cells were stained with BTX₆₄₇ as above. Images were captured on a Nikon A1RMP confocal microscope with a 40× oil immersion objective.

### Data and Statistical Analyses

Data were analyzed off-line using FlowJo, PulseFit (HEKA), Microsoft Excel, Origin and GraphPad Prism software. Statistical analyses were performed in Origin or GraphPad Prism using built-in functions. Statistically significant differences between means (p<0.05) were determined using one-way ANOVA with Tukey’s multiple comparison test or two-tailed unpaired t test for comparisons between two groups. Data are presented as means ± s.e.m unless otherwise noted.

### Example 2

### RESTORx: a Next-Generation Therapeutic Modality Based on Targeted Protein Stabilization

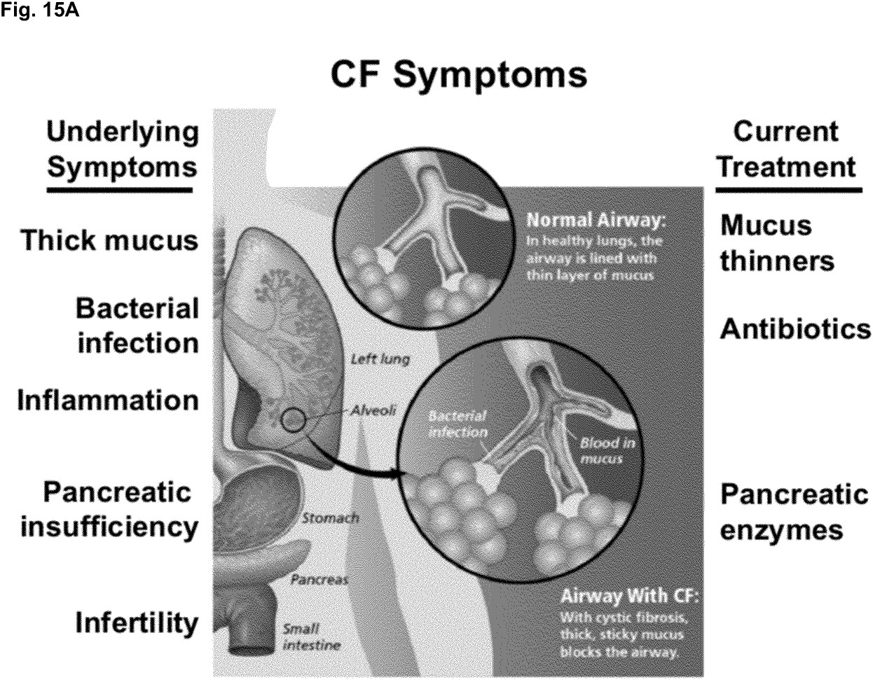

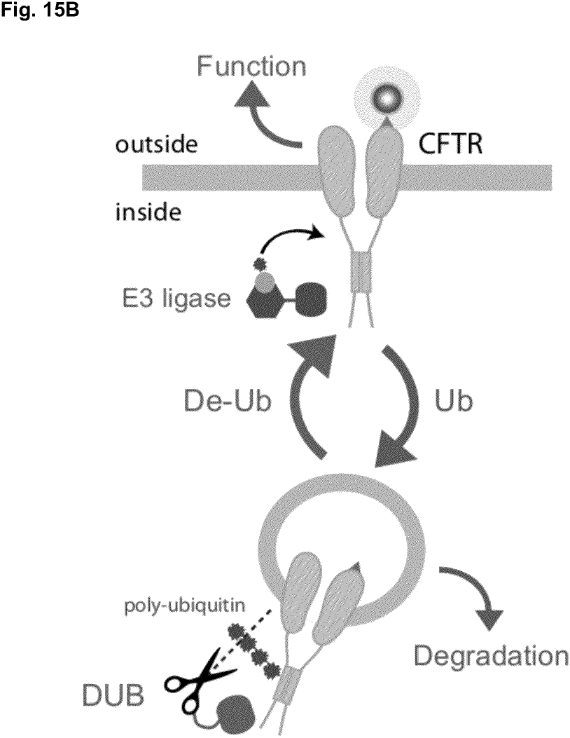

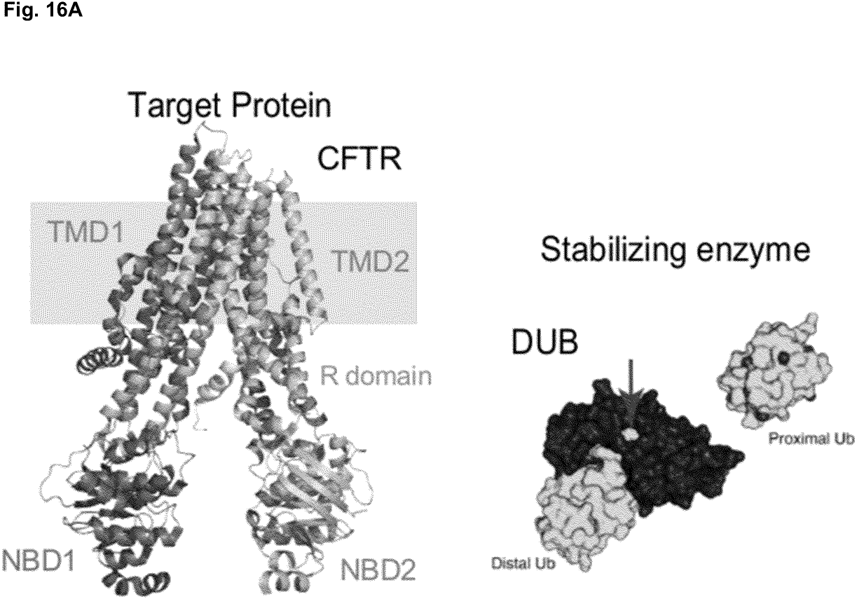

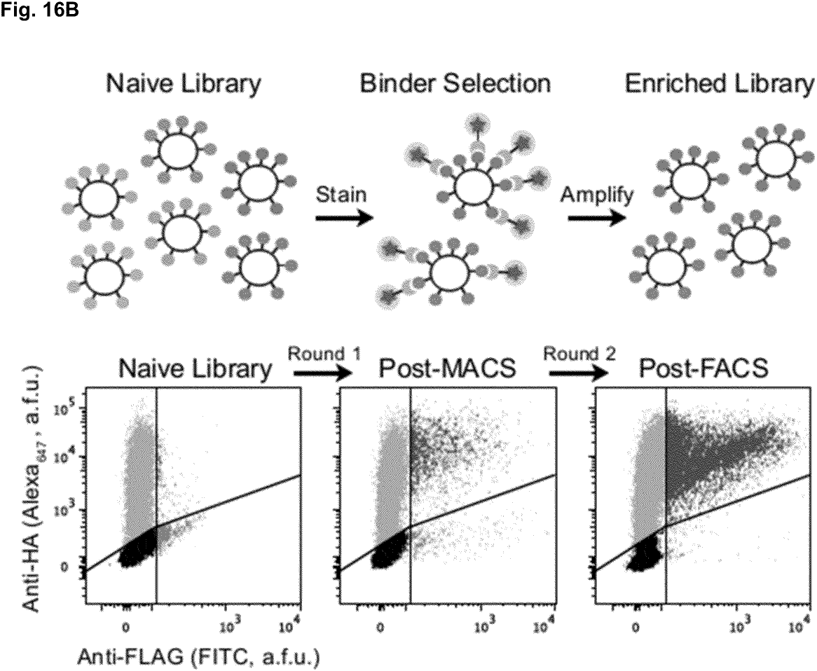

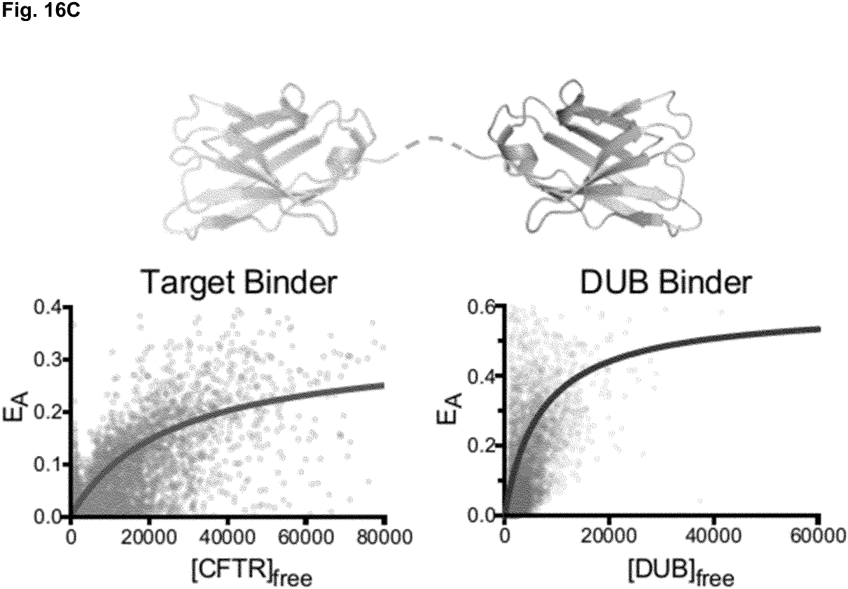

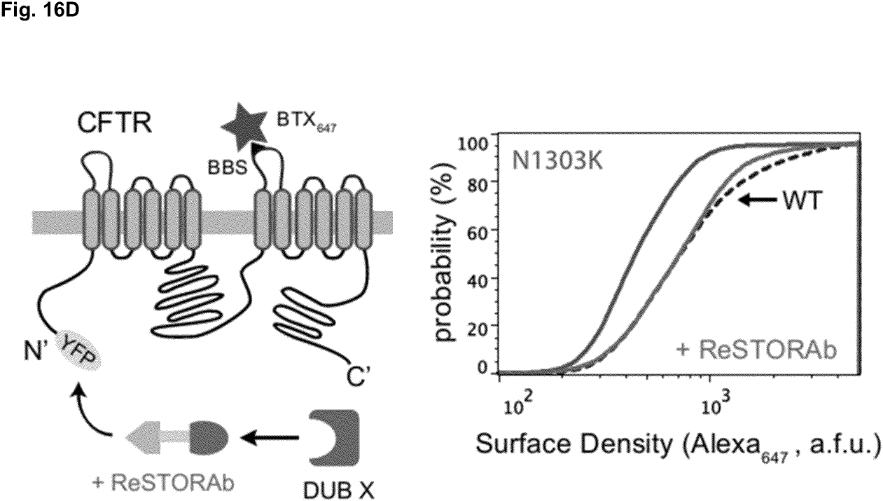

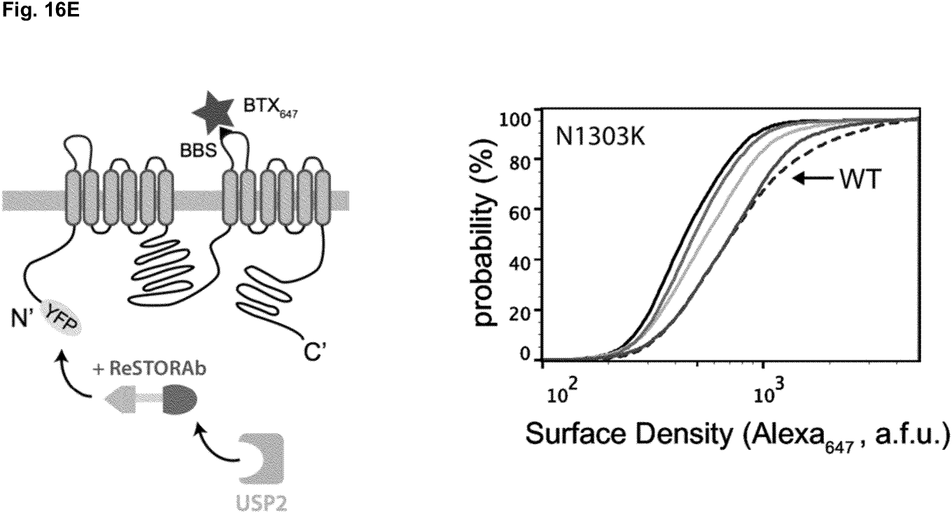

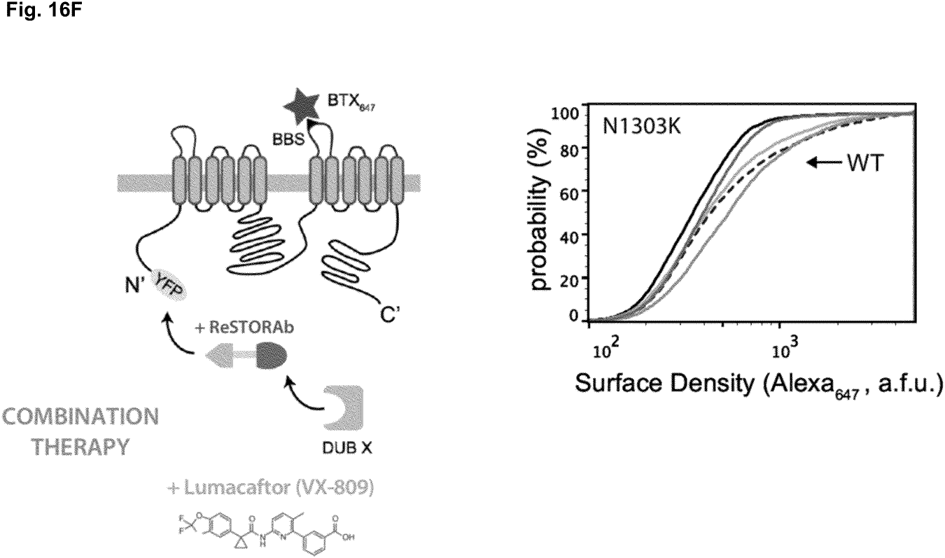

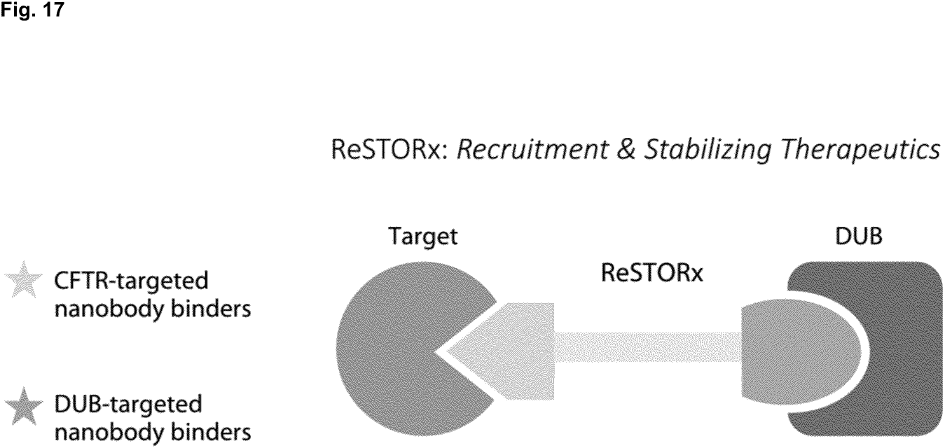

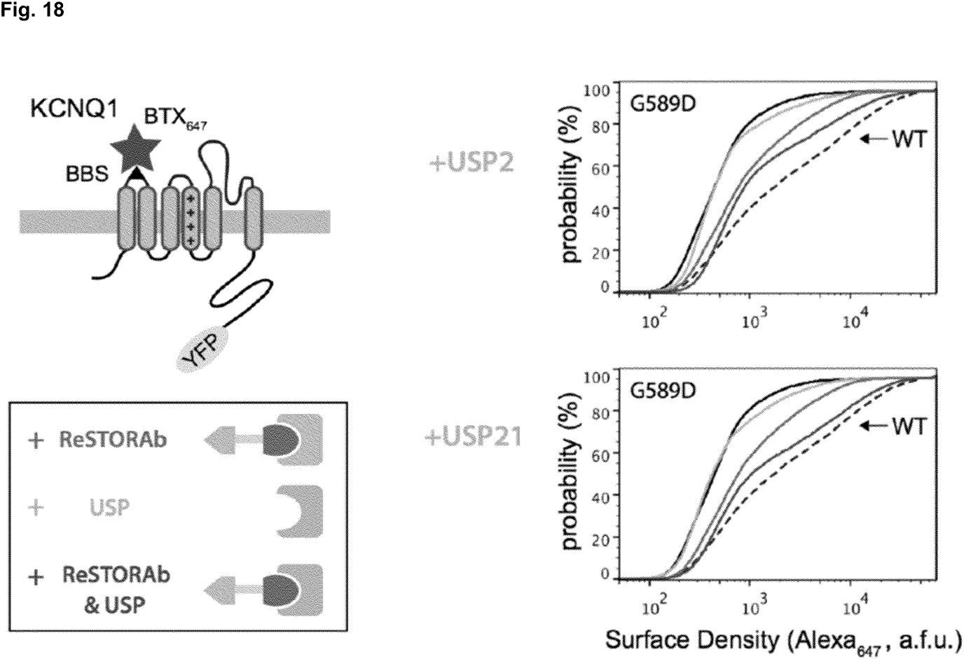

Protein stability is a key point of regulation for all proteins in the cell. Ubiquitination plays a major role in intracellular protein homeostasis, and dysregulation of this process can lead to the pathogenesis of many diseases. The present disclosure focuses on cystic fibrosis (CF), a rare, inherited disease with high unmet need, as the primary indication. Although the vast majority of CF mutations lead to deficits in the stability of a chloride channel, CFTR, the current gold standard treatments are overwhelmingly symptom based: lung airway clearance techniques, inhalation of mucus thinners, and antibiotic treatment of bacterial infections (FIGS. 15A-15B). While these treatments have improved life expectancy (~30-40 years old), there remains no definitive treatment and CF patients continue to experience rapidly deteriorating quality of life. Only recently has there been a push in the development of pharmacologic chaperones, or ‘correctors’, that look to promote mutant CFTR trafficking to the cell membrane; however, to date, the clinical efficacy of such treatments has been relatively modest with many mutations remaining resistant to therapy.

The present disclosure took an entirely distinct small-molecule approach for the rescue of CFTR trafficking and stability (FIG. 16A). In particular, the goal was to exploit the powerful, yet reversible nature of ubiquitination with a novel hypothesis: could we recruit endogenous deubiquitinases (DUBs) to mutant CFTR channels in order to selectively tune the ubiquitin status, enhance channel stability, and restore function? We term this general approach Rescue & Stabilization on Redirection of Endogenous DUBs (ReSTORED), and resulting molecules that exploit this mechanism, called Rescue and Stabilization Therapeutics (ReSTORx). Fundamentally, our ReSTORx are heterobifunctional molecules comprised of 3 distinct modules: 1) a DUBbinding molecule, 2) a target-binding molecule, and 3) a variable linker joining the two. As such, our ReSTORx compounds act as molecular bridges, joining endogenous DUB activity to a target protein-of-interest. To test this novel approach, we first developed nanobody-based binders for both DUB and CFTR proteins using a yeast surface display library (FIG. 16B; Table 1). The resulting ReSTORx molecule, a bivalent nanobody-based ReSTORAb (FIG. 17), was able to bind both proteins inside living cells and significantly rescued mutant CFTR surface trafficking up to WT levels (FIGS. 16C- 16F). Further, it has been shown that the bivalent nanobody-based ReSTORAb was able to rescue LQTS trafficking deficits (FIG. 18).

TABLE 1

Nanobody-based binders for both DUB and CFTR proteins.
Nanobody clones
DUB (USP) targeted nanobody clones

| | (CAAS.x xx.GWY R) | (GKER.xx x.ADSV) | (VYYC.xxx. WGQG) | |
|---|---|---|---|---|
| nanobody | CDR1 | CDR2 | CDR3 | Full Sequence |
| A3 | GTIFAT YYM (SEQ ID No: 7) | ELVAAIA YGGTTY Y (SEQ ID No: 8) | AAEQYEQ YRTLPPYD Y (SEQ ID No: 9) | QVQLQESGGGLVQAGGSLRLSCA ASGTIFATYYMGWYRQAPGKEREL VAAIAYGGTTYYADSVKGRFTISRD NAKNTVYLQMNSLKPEDTAVYYCA AEQYEQYRTLPPYDYWGQGTQVT VSS (SEQ ID No: 1) |
| A5 | GYIFGI VYM (SEQ ID No: 10) | ELVATID TGTNTYY (SEQ ID No: 11) | AAEGRDY RDYDY (SEQ ID No: 12) | QVQLQESGGGLVQAGGSLRLSCA ASGYIFGIVYMGWYRQAPGKEREL VATIDTGTNTYYADSVKGRFTISRD NAKNTVYLQMNSLKPEDTAVYYCA AEGRDYRDYDYWGQGTQVTVSS (SEQ ID No: 2) |
| B3 | GTISDT RYM (SEQ ID No: 13) | ELVAAID YGSTTYY (SEQ ID No: 14) | AAEYVLSK DHEY (SEQ ID No: 15) | QVQLQESGGGLVQAGGSLRLSCA ASGTISDTRYMGWYRQAPGKERE LVAAIDYGSTTYYADSVKGRFTISR DNAKNTVYLQMNSLKPEDTAVYYC |
| | | | | AAEYVLSKDHEYWGQGTQVTVSS (SEQ ID No: 3) |
| B5 | GSIFER AYM (SEQ ID No: 16) | EFVAAIG YGTNTN Y (SEQ ID No: 17) | AALARDVY SYNY (SEQ ID No: 18) | QVQLQESGGGLVQAGGSLRLSCA ASGSIFERAYMGWYRQAPGKERE FVAAIGYGTNTNYADSVKGRFTISR DNAKNTVYLQMNSLKPEDTAVYYC AALARDVYSYNYWGQGTQVTVSS (SEQ ID No: 4) |
| D9 | GTIFSF SYM (SEQ ID No: 19) | ELVAAIA RGTTTYY (SEQ ID No: 20) | AAEHNWG EPYRSYYD Y (SEQ ID No: 21) | QVQLQESGGGLVQAGGSLRLSCA ASGTIFSFSYMGWYRQAPGKEREL VAAIARGTTTYYADSVKGRFTISRD NAKNTVYLQMNSLKPEDTAVYYCA AEHNWGEPYRSYYDYWGQGTQV TVSS (SEQ ID No: 5) |
| H10 | GYISDY LRM (SEQ ID No: 22) | ELVATIA RGGITNY (SEQ ID No: 23) | AARLPYYK YNGFVLVY (SEQ ID No: 24) | QVQLQESGGGLVQAGGSLRLSCA ASGYISDYLRMGWYRQAPGKERE LVATIARGGITNYADSVKGRFTISR DNAKNTVYLQMNSLKPEDTAVYYC AARLPYYKYNGFVLVYWGQGTQV TVSS (SEQ ID No: 6) |
| CFTR (NBD1) target nanobody clones | | | | |
| | (CAAS.x xx.GWY R) | (GKER.xx x.ADSV) | (VYYC.xxx. WGQG) | |
| nanobody | CDR1 | CDR2 | CDR3 | Full Sequence |
| E3h | GTISGS GSM (SEQ ID No: 39) | EFVAAIN VGSNTY Y (SEQ ID No: 40) | AVRFGYYY RHTY (SEQ ID No: 41) | QVQLQESGGGLVQAGGSLRLSCA ASGTISGSGSMGWYRQAPGKERE FVAAINVGSNTYYADSVKGRFTISR DNAKNTVYLQMNSLKPEDTAVYYC AVRFGYYYRHTYWGQGTQVTVSS (SEQ ID No: 25) |
| E8h | GSIFSR FYM (SEQ ID No: 42) | EFVAGIS AGGTTY Y (SEQ ID No: 43) | AWAGRLL RYRY (SEQ ID No: 44) | QVQLQESGGGLVQAGGSLRLSCA ASGSIFSRFYMGWYRQAPGKERE FVAGISAGGTTYYADSVKGRFTISR DNAKNTVYLQMNSLKPEDTAVYYC AVVAGRLLRYRYWGQGTQVTVSS (SEQ ID No: 26) |
| E11h | GTISYH GTM (SEQ ID No: 45) | EFVAAIA RGGNTN Y (SEQ ID No: 46) | AALLRRSG YITSSFLY (SEQ ID No: 47) | QVQLQESGGGLVQAGGSLRLSCA ASGTISYHGTMGWYRQAPGKERE FVAAIARGGNTNYADSVKGRFTISR DNAKNTVYLQMNSLKPEDTAVYYC AALLRRSGYITSSFLYWGQGTQVT VSS (SEQ ID No: 27) |
| A5h | GTISRY TTM (SEQ ID No: 48) | ELVAGIT PGGSTY Y (SEQ ID No: 49) | AARDYWA KLSY (SEQ ID No: 50) | QVQLQESGGGLVQAGGSLRLSCA ASGTISRYTTMGWYRQAPGKEREL VAGITPGGSTYYADSVKGRFTISRD NAKNTVYLQMNSLKPEDTAVYYCA ARDYWAKLSYWGQGTQVTVSS (SEQ ID No: 28) |
| C2h | GSIFSR TSM (SEQ ID No: 51) | ELVAGIT WGGNTY Y (SEQ ID No: 52) | AVLVPIGR DVKGYHR Y (SEQ ID No: 53) | QVQLQESGGGLVQAGGSLRLSCA ASGSIFSRTSMGWYRQAPGKERE LVAGITWGGNTYYADSVKGRFTIS RDNAKNTVYLQMNSLKPEDTAVYY CAVLVPIGRDVKGYHRYWGQGTQ VTVSS (SEQ ID No: 29) |
| C11h | GTIFRY AVM (SEQ ID No: 54) | EFVAAIN SGTNTN Y (SEQ ID No: 55) | AALYRNPA FPIYAHTY (SEQ ID No: 56) | QVQLQESGGGLVQAGGSLRLSCA ASGTIFRYAVMGWYRQAPGKERE FVAAINSGTNTNYADSVKGRFTISR DNAKNTVYLQMNSLKPEDTAVYYC AALYRNPAFPIYAHTYWGQGTQVT VSS (SEQ ID No: 30) |
| F4h | GTIFSY GYMG (SEQ ID No: 57) | EFVAGIS RGATTN Y (SEQ ID No: 58) | AWGLRV QYQAYLY RY (SEQ ID No: 59) | QVQLQESGGGLVQAGGSLRLSCA ASGTIFSYGYMGWYRQAPGKERE FVAGISRGATTNYADSVKGRFTISR DNAKNTVYLQMNSLKPEDTAVYYC AWGLRVQYQAYLYRYWGQGTQV TVSS (SEQ ID No: 31) |
| A7L | GSISRF GVM (SEQ ID No: 60) | EFVAAIA SGTTTYY (SEQ ID No: 61) | AAREYGY GGHLY (SEQ ID No: 62) | QVQLQESGGGLVQAGGSLRLSCA ASGSISRFGVMGWYRQAPGKERE FVAAIASGTTTYYADSVKGRFTISR DNAKNTVYLQMNSLKPEDTAVYYC AAREYGYGGHLYWGQGTQVTVSS (SEQ ID No: 32) |
| B8L | GSIFYY SRM (SEQ ID No: 63) | ELVAGIG RGTTYY (SEQ ID No: 64) | AVYPNYQ WAYAVLH GY (SEQ ID No: 65) | QVQLQESGGGLVQAGGSLRLSCA ASGSIFYYSRMGWYRQAPGKERE LVAGIGRGTTYYADSVKGRFTISRD NAKNTVYLQMNSLKPEDTAVYYCA VYPNYQWAYAVLHGYWGQGTQV TVSS (SEQ ID No: 33) |
| C6L | GSISYY LYM (SEQ ID No: 66) | EFVAAIN RGATTYY (SEQ ID No: 67) | AVRAIQTS SERRYFTY (SEQ ID No: 68) | QVQLQESGGGLVQAGGSLRLSCA ASGSISYYLYMGWYRQAPGKERE FVAAINRGATTYYADSVKGRFTISR DNAKNTVYLQMNSLKPEDTAVYYC AVRAIQTSSERRYFTYWGQGTQVT VSS (SEQ ID No: 34) |
| D9L | GTISLA RYM | EFVAGIT YGTTTYY | AAYLRSTT SGYLYHRY | QVQLQESGGGLVQAGGSLRLSCA ASGTISLARYMGWYRQAPGKERE FVAGITYGTTTYYADSVKGRFTISR |
| | (SEQ ID No: 69) | (SEQ ID No: 70) | (SEQ ID No: 71) | DNAKNTVYLQMNSLKPEDTAVYYC AAYLRSTTSGYLYHRYWGQGTQV TVSS (SEQ ID No: 35) |
| E3L | GTVSY AM (SEQ ID No: 72) | EFVAAITL GSNTNY (SEQ ID No: 73) | AAYRRYG KTLYLLY (SEQ ID No: 74) | QVQLQESGGGLVQAGGSLRLSCA ASGTVSYAMGWYRQAPGKEREFV AAITLGSNTNYADSVKGRFTISRDN AKNTVYLQMNSLKPEDTAVYYCAA YRRYGKTLYLLYWGQGTQVTVSS (SEQ ID No: 36) |
| E8L | GTISSD AWM (SEQ ID No: 75) | ELVASIS TGATTYY (SEQ ID No: 76) | AAVPRRR GYYTYYFR Y (SEQ ID No: 77) | QVQLQESGGGLVQAGGSLRLSCA ASGTISSDAWMGWYRQAPGKERE LVASISTGATTYYADSVKGRFTISR DNAKNTVYLQMNSLKPEDTAVYYC AAVPRRRGYYTYYFRYWGQGTQV TVSS (SEQ ID No: 37) |
| F7L | GYIFQY ASM (SEQ ID No: 78) | ELVAGIS AGATTYY (SEQ ID No: 79) | AARWYDL SQYPRRH HY (SEQ ID No: 80) | QVQLQESGGGLVQAGGSLRLSCA ASGYIFQYASMGWYRQAPGKERE LVAGISAGATTYYADSVKGRFTISR DNAKNTVYLQMNSLKPEDTAVYYC AARWYDLSQYPRRHHYWGQGTQ VTVSS (SEQ ID No: 38) |

The ReSTORx technology emerges as a first-in-class CFTR stabilizer, distinct from any therapeutics on market or in development for CF, and rationally designed for targeted ubiquitin removal from mutant channels. Its unique mechanism-of-action promotes synergistic efficacy with current modulators, and rescues previously unresponsive CFTR mutations. Furthermore, the modular nature of the ReSTORx technology suggests a highly adaptable, protein stabilizing platform. As such, the “active” DUB-recruiting components can be readily adapted for use with any given target-binding molecule, with the potential for improving the efficacy of currently marketed drugs or functionalizing previously quiescent compounds that engage a target without therapeutic effect.

The potential impact of such a ReSTORx platform extends into the ubiquitin therapeutic space. Competition in ubiquitin therapeutics has been mainly confined to nonselective inhibitors of the ubiquitin proteasome system (UPS). Proteasome inhibitors have had large commercial success, for example, the first-to-market UPS modulator, Velcade® (bortezomib), generated $3 billion USD revenue in 2014 alone; however, since these drugs target the entire protein degradation pathway, lack of target specificity has restricted their use and led to significant side effects in patients. Consequently, the focus is gradually shifting from proteasome inhibitors to targeting specific components of the UPS (i.e. E3 ubiquitin ligases). However, even these ubiquitin enzymes suffer from promiscuity in the regulation of many different substrates. In contrast, the ReSTORx molecules disclosed herein enjoy both specificity in targeting and generalizability in action, exploiting a huge unmet market need for selective UPS modulators. This entirely new therapeutic modality can further expand indications to other inherited channelopathies and cancer therapeutics.

### DOCUMENTS CITED

The following references, to the extent that they provide exemplary procedural or other details supplementary to those set forth herein, are specifically incorporated herein by reference.

1. Kullmann, D. M. Neurological Channelopathies. Annual Review of Neuroscience 33, 151-172, doi:10.1146/annurev-neuro-060909-153122 (2010).

2. Bohnen, M. S. et al. Molecular Pathophysiology of Congenital Long QT Syndrome. Physiological Reviews 97, 89-134, doi:10.1152/physrev.00008.2016 (2016).

3. Cutting, G. R. Cystic fibrosis genetics: from molecular understanding to clinical application. Nature Reviews Genetics 16, 45-56, doi:10.1038/nrg3849 (2014).

4. Curran, J. & Mohler, P. J. Alternative Paradigms for Ion Channelopathies: Disorders of Ion Channel Membrane Trafficking and Posttranslational Modification. Annual Review of Physiology 77, 1-20, doi:10.1146/annurev-physiol-021014-071838 (2015).

5. Foot, N., Henshall, T. & Kumar, S. Ubiquitination and the Regulation of Membrane Proteins. Physiol Rev 97, 253-281, doi:10.1152/physrev.00012.2016 (2017).

6. MacGurn, J. A., Hsu, P. C. & Emr, S. D. Ubiquitin and membrane protein turnover: from cradle to grave. Annu Rev Biochem 81, 231-259, doi:10.1146/annurevbiochem-060210-093619 (2012).

7. Ashcroft, F. M. & Rorsman, P. KATP channels and islet hormone secretion: new insights and controversies. Nature Reviews Endocrinology 9, 660, doi:10.1038/nrendo.2013.166 (2013).

8. Imbrici, P. et al. Therapeutic Approaches to Genetic Ion Channelopathies and Perspectives in Drug Discovery. Frontiers in Pharmacology 7, 121, doi:10.3389/fphar.2016.00121 (2016).

9. Wulff, H., Christophersen, P., Colussi, P., Chandy, G. K. & Yarov-Yarovoy, V. Antibodies and venom peptides: new modalities for ion channels. Nature Reviews Drug Discovery, 1, doi:10.1038/s41573-019-0013-8 (2019).

10. Tester, D. J., Will, M. L., Haglund, C. M. & Ackerman, M. J. Compendium of cardiac channel mutations in 541 consecutive unrelated patients referred for long QT syndrome genetic testing. Heart rhythm 2, 507-517, doi:10.1016/j.hrthm.2005.01.020 (2005).

11. Wilson, A. J., Quinn, K. V., Graves, F. M., Bitner-Glindzicz, M. & Tinker, A. Abnormal KCNQ1 trafficking influences disease pathogenesis in hereditary long QT syndromes (LQT1). Cardiovascular Research 67, 476-486, doi:10.1016/j.cardiores.2005.04.036 (2005).

12. Haardt, M., Benharouga, M., Lechardeur, D., Kartner, N. & Lukacs, G. L. C-terminal truncations destabilize the cystic fibrosis transmembrane conductance regulator without impairing its biogenesis. A novel class of mutation. The Journal of biological chemistry 274, 21873-21877, doi:10.1074/jbc.274.31.21873 (1999).

13. Cheng, S. H. et al. Defective intracellular transport and processing of CFTR is the molecular basis of most cystic fibrosis. Cell 63, 827-834 (1990).

14. Nalepa, G., Rolfe, M. & Harper, W. J. Drug discovery in the ubiquitin-proteasome system. Nature Reviews Drug Discovery 5, 596-613, doi:10.1038/nrd2056 (2006).

15. Huang, X. & Dixit, V. M. Drugging the undruggables: exploring the ubiquitin system for drug development. Cell Research 26, 484-498, doi:10.1038/cr.2016.31 (2016).

16. Jespersen, T. et al. The KCNQ1 potassium channel is down-regulated by ubiquitylating enzymes of the Nedd4/Nedd4-like family. Cardiovasc Res 74, 64-74, doi:10.1016/j.cardiores.2007.01.008 (2007).

17. Mevissen, T. et al. OTU Deubiquitinases Reveal Mechanisms of Linkage Specificity and Enable Ubiquitin Chain Restriction Analysis. Cell 154, 169-184, doi:10.1016/j.cell.2013.05.046 (2013).

18. Rothbauer, U. et al. A versatile nanotrap for biochemical and functional studies with fluorescent fusion proteins. Molecular & cellular proteomics: MCP 7, 282-289, doi:10.1074/mcp.M700342-MCP200 (2008).

19. Aromolaran, A. S., Subramanyam, P., Chang, D. D., Kobertz, W. R. & Colecraft, H. M. LQT1 mutations in KCNQ1 C-terminus assembly domain suppress IKs using different mechanisms. Cardiovasc Res 104, 501-511, doi:10.1093/cvr/cvu231 (2014).

20. Peroz, D., Dahimène, S., Baró, I., Loussouarn, G. & Mérot, J. LQT1-associated Mutations Increase KCNQ1 Proteasomal Degradation Independently of Derlin-1. Journal of Biological Chemistry 284, 5250-5256, doi:10.1074/jbc.M806459200 (2009).

21. Mattmann, M. E. et al. Identification of (R)-N-(4-(4-methoxyphenyl)thiazol-2-yl)-1-tosylpiperidine-2-carboxamide, ML277, as a novel, potent and selective Kv7.1 (KCNQ1) potassium channel activator. Bioorganic & Medicinal Chemistry Letters 22, 5936-5941, doi:10.1016/j.bmcl.2012.07.060 (2012).

22. Veit, G. et al. From CFTR biology toward combinatorial pharmacotherapy: expanded classification of cystic fibrosis mutations. Molecular biology of the cell 27, 424-433, doi:10.1091/mbc.E14-04-0935 (2016).

23. Boeck, K. & Amaral, M. D. Progress in therapies for cystic fibrosis. The Lancet Respiratory Medicine 4, 662-674, doi:10.1016/S2213-2600(16)00023-0 (2016).

24. Wainwright, C. E. et al. Lumacaftor-Ivacaftor in Patients with Cystic Fibrosis Homozygous for Phe508del CFTR. The New England Journal of Medicine 373, 220-231, doi:10.1056/NEJMoa1409547 (2015).

25. Goor, F. et al. Correction of the F508del-CFTR protein processing defect in vitro by the investigational drug VX-809. Proceedings of the National Academy of Sciences 108, 18843-18848, doi:10.1073/pnas.1105787108 (2011).

26. Goor, F. et al. Rescue of CF airway epithelial cell function in vitro by a CFTR potentiator, VX-770. Proceedings of the National Academy of Sciences 106, 18825-18830, doi:10.1073/pnas.0904709106 (2009).

27. Farinha, C. M. & Matos, P. Repairing the basic defect in cystic fibrosis - one approach is not enough. FEBS Journal 283, 246-264, doi:10.1111/febs.13531 (2016).

28. Faesen, A. C. et al. The differential modulation of USP activity by internal regulatory domains, interactors and eight ubiquitin chain types. Chem Biol 18, 1550-1561, doi:10.1016/j.chembiol.2011.10.017 (2011).

29. McMahon, C. et al. Yeast surface display platform for rapid discovery of conformationally selective nanobodies. Nature Structural & Molecular Biology 25, 289-296, doi:10.1038/s41594-018-0028-6 (2018).

30. Galietta, L. V., Jayaraman, S. & Verkman, A. S. Cell-based assay for highthroughput quantitative screening of CFTR chloride transport agonists. American journal of physiology. Cell physiology 281, 42, doi:10.1152/ajpcell.2001.281.5.C1734 (2001).

31. Yu, H. et al. Ivacaftor potentiation of multiple CFTR channels with gating mutations. Journal of Cystic Fibrosis 11, 237-245, doi:10.1016/j.jcf.2011.12.005 (2012).

32. Ratner, M. FDA deems in vitro data on mutations sufficient to expand cystic fibrosis drug label. Nature Biotechnology 35, 606-606, doi:10.1038/nbt0717-606 (2017).

33. Durmowicz, A. G., Lim, R., Rogers, H., Rosebraugh, C. J. & Chowdhury, B. A. The U.S. Food and Drug Administration’s Experience with Ivacaftor in Cystic Fibrosis. Establishing Efficacy Using In Vitro Data in Lieu of a Clinical Trial. Annals of the American Thoracic Society 15, 1-2, doi:10.1513/AnnalsATS.201708-668PS (2018).

34. Han, S. T. et al. Residual function of cystic fibrosis mutants predicts response to small molecule CFTR modulators. JCI Insight 3, doi:10.1172/jci.insight. 121159 (2018).

35. Goor, F., Yu, H., Burton, B. & Hoffman, B. J. Effect of ivacaftor on CFTR forms with missense mutations associated with defects in protein processing or function. Journal of Cystic Fibrosis 13, 29-36, doi:10.1016/j.jcf.2013.06.008 (2014).

36. Lukacs, G. L. & Verkman, A. S. CFTR: folding, misfolding and correcting the ΔF508 conformational defect. Trends in Molecular Medicine 18, 81-91, doi:10.1016/j.molmed.2011.10.003 (2012).

37. Okiyoneda, T. et al. Mechanism-based corrector combination restores ΔF508-CFTR folding and function. Nature Chemical Biology 9, 444-454, doi:10.1038/nchembio.1253 (2013).

38. Okiyoneda, T. et al. Peripheral protein quality control removes unfolded CFTR from the plasma membrane. Science (New York, N.Y.) 329, 805-810, doi:10.1126/science.1191542 (2010).

39. Sigoillot, M. et al. Domain-interface dynamics of CFTR revealed by stabilizing nanobodies. Nature Communications 10, doi:ARTN 263610.1038/s41467-019-10714-y (2019).

40. Delisle, B. P. et al. Biology of Cardiac Arrhythmias. Circulation Research 94, 1418-1428, doi:10.1161/01.RES.0000128561.28701.ea (2004).

41. Liu, F., Zhang, Z., Csanády, L., Gadsby, D. C. & Chen, J. Molecular Structure of the Human CFTR Ion Channel. Cell 169, 85-910065408, doi:10.1 016/j.cell.2017.02.024 (2017).

42. Kanner, S. A., Morgenstern, T. & Colecraft, H. M. Sculpting ion channel functional expression with engineered ubiquitin ligases. eLife 6, doi:10.7554/eLife.29744 (2017).

43. Fridy, P. C. et al. A robust pipeline for rapid production of versatile nanobody repertoires. Nature Methods 11, 1253-1260, doi:10.1038/nmeth.3170 (2014).

44. Sekine-Aizawa, Y. & Huganir, R. L. Imaging of receptor trafficking by using alphabungarotoxin-binding-site-tagged receptors. Proc Natl Acad Sci U S A 101, 17114-17119, doi:10.1073/pnas.0407563101 (2004).

45. Gao, S. et al. Ubiquitin ligase Nedd4L targets activated Smad⅔ to limit TGF-beta signaling. Mol Cell 36, 457-468, doi:10.1016/j.molcel.2009.09.043 (2009).

46. Peters, K. W. et al. CFTR Folding Consortium: methods available for studies of CFTR folding and correction. Methods MolBiol 742, 335-353, doi:10.1007/978-1-61779-120-8_20 (2011).

47. Galietta, L. J., Haggie, P. M. & Verkman, A. S. Green fluorescent protein-based halide indicators with improved chloride and iodide affinities. FEBS letters 499, 220-224, doi:10.1016/s0014-5793(01)02561-3 (2001).

48. Kanner, S. A., Jain, A. & Colecraft, H. M. Development of a High-Throughput Flow Cytometry Assay to Monitor Defective Trafficking and Rescue of Long QT2 Mutant hERG Channels. Frontiers in Physiology 9, 397, doi:10.3389/fphys.2018.00397 (2018).

49. Lee, S.-R., Sang, L. & Yue, D. T. Uncovering Aberrant Mutant PKA Function with Flow Cytometric FRET. Cell Reports 14, 3019-3029, doi:10.1016/j.celrep.2016.02.077 (2016).

All patents, patent applications, and publications cited herein are incorporated herein by reference in their entirety as if recited in full herein.

The disclosure being thus described, it will be obvious that the same may be varied in many ways. Such variations are not to be regarded as a departure from the spirit and scope of the disclosure and all such modifications are intended to be included within the scope of the following claims.

1. A bivalent molecule comprising:
a) a deubiquitinase (DUB) binder;
b) a target binder; and
c) a variable linker between the DUB binder and the target binder, wherein the DUB binder comprises a nanobody, scFv, antibody mimetic, monobody, DARPin, lipocalin, targeting sequence, or intracellular antibody.

2. The bivalent molecule of claim 1, wherein the DUB is endogenous.

3. The bivalent molecule of claim 1, wherein the DUB is a ubiquitin specific proteases (USP) family member, ovarian tumor proteases (OTU) family member, ubiquitin C-terminal hydrolases (UCH) family member, Josephin domain (Josephin) family member, motif interacting with ubiquitin-containing novel DUB (MINDY) family member, or JAB1/MPN/Mov34 metalloenzyme domain (JAMM) family member.

4. (canceled)

5. The bivalent molecule of claim 1, wherein the DUB binder is a nanobody.

6-8. (canceled)

9. The bivalent molecule of claim 5, wherein the amino acid sequence of the nanobody comprises the amino acid sequence set forth as any one of SEQ ID NOs: 1 to 6.

10. The bivalent molecule of claim 5, wherein the nanobody comprises a complementarity determining region (CDR) 1, CDR2, and CDR3, and wherein:
a) the amino acid sequence of CDR1 comprises the amino acid sequence set forth as SEQ ID No: 7, a the amino acid sequence of CDR2 comprises the amino acid sequence set forth as SEQ ID No: 8, and a the amino acid sequence of CDR3 comprises the amino acid sequence set forth as SEQ ID No: 9;
b) a the amino acid sequence of CDR1 comprises the amino acid sequence set forth as SEQ ID No: 10, the amino acid sequence of CDR2 comprises the amino acid sequence set forth as SEQ ID No: 11, and a the amino acid sequence of CDR3 comprises the amino acid sequence set forth as SEQ ID No: 12;
c) a the amino acid sequence of CDR1 comprises the amino acid sequence set forth as SEQ ID No: 13, the amino acid sequence of CDR2 comprises the amino acid sequence set forth as SEQ ID No: 14, and a the amino acid sequence of CDR3 comprises the amino acid sequence set forth as SEQ ID No: 15;
d) a the amino acid sequence of CDR1 comprises the amino acid sequence set forth as SEQ ID No: 16, the amino acid sequence of CDR2 comprises the amino acid sequence set forth as SEQ ID No: 17, and a the amino acid sequence of CDR3 comprises the amino acid sequence set forth as SEQ ID No: 18;
e) the amino acid sequence of CDR1 comprises the amino acid sequence set forth as SEQ ID No: 19, the amino acid sequence of CDR2 comprises the amino acid sequence set forth as SEQ ID No: 20, and a the amino acid sequence of CDR3 comprises the amino acid sequence set forth as SEQ ID No: 21; or
f) the amino acid sequence of CDR1 comprises the amino acid sequence set forth as SEQ ID No: 22, a the amino acid sequence of CDR2 comprises the amino acid sequence set forth as SEQ ID No: 23, and a the amino acid sequence of CDR3 comprises the amino acid sequence set forth as SEQ ID No: 24.

11-16. (canceled)

17. The bivalent molecule of claim 1, wherein the target binder comprises a nanobody, scFv, antibody mimetic, monobody, DARPin, lipocalin, targeting sequence, or intracellular antibody.

18. The bivalent molecule of claim 1, wherein the target binder is a nanobody.

19. (canceled)

20. The bivalent molecule of claim 18, wherein the amino acid sequence of the nanobody comprises the amino acid sequence set forth as any one of SEQ ID NOs: 25 to 38.

21. The bivalent molecule of claim 18, wherein the nanobody comprises a CDR1, CDR2, and CDR3, and wherein:
a) the amino acid sequence of CDR1 comprises the amino acid sequence set forth as SEQ ID No: 39, a the amino acid sequence of CDR2 comprises the amino acid sequence set forth as SEQ ID No: 40, and the amino acid sequence of CDR3 comprises the amino acid sequence set forth as SEQ ID No: 41;
b) a the amino acid sequence of CDR1 comprises the amino acid sequence set forth as SEQ ID No: 42, a the amino acid sequence of CDR2 comprises the amino acid sequence set forth as SEQ ID No: 43, and a the amino acid sequence of CDR3 comprises the amino acid sequence set forth as SEQ ID No: 44;
c) a the amino acid sequence of CDR1 comprises the amino acid sequence set forth as SEQ ID No: 45, a the amino acid sequence of CDR2 comprises the amino acid sequence set forth as SEQ ID No: 46, and a the amino acid sequence of CDR3 comprises the amino acid sequence set forth as SEQ ID No: 47;
d) a the amino acid sequence of CDR1 comprises the amino acid sequence set forth as SEQ ID No: 48, a the amino acid sequence of CDR2 comprises the amino acid sequence set forth as SEQ ID No: 49, and a the amino acid sequence of CDR3 comprises the amino acid sequence set forth as SEQ ID No: 50;
e) the amino acid sequence of CDR1 comprises the amino acid sequence set forth as SEQ ID No: 51, the amino acid sequence of CDR2 comprises the amino acid sequence set forth as SEQ ID No: 52, and a the amino acid sequence of CDR3 comprises the amino acid sequence set forth as SEQ ID No: 53;
f) the amino acid sequence of CDR1 comprises the amino acid sequence set forth as SEQ ID No: 54, the amino acid sequence of CDR2 comprises the amino acid sequence set forth as SEQ ID No: 55, and a the amino acid sequence of CDR3 comprises the amino acid sequence set forth as SEQ ID No: 56;
g) the amino acid sequence of CDR1 comprises the amino acid sequence set forth as SEQ ID No: 57, a the amino acid sequence of CDR2 comprises the amino acid sequence set forth as SEQ ID No: 58, and a the amino acid sequence of CDR3 comprises the amino acid sequence set forth as SEQ ID No: 59;
h) the amino acid sequence of CDR1 comprises the amino acid sequence set forth as SEQ ID No: 60, a the amino acid sequence of CDR2 comprises the amino acid sequence set forth as SEQ ID No: 61, and a the amino acid sequence of CDR3 comprises the amino acid sequence set forth as SEQ ID No: 62;
i) the amino acid sequence of CDR1 comprises the amino acid sequence set forth as SEQ ID No: 63, the amino acid sequence of CDR2 comprises the amino acid sequence set forth as SEQ ID No: 64, and a the amino acid sequence of CDR3 comprises the amino acid sequence set forth as SEQ ID No: 65;
j) the amino acid sequence of CDR1 comprises the amino acid sequence set forth as SEQ ID No: 66, the amino acid sequence of CDR2 comprises the amino acid sequence set forth as SEQ ID No: 67, and a the amino acid sequence of CDR3 comprises the amino acid sequence set forth as SEQ ID No: 68;
k) a the amino acid sequence of CDR1 comprises the amino acid sequence set forth as SEQ ID No: 69, a the amino acid sequence of CDR2 comprises the amino acid sequence set forth as SEQ ID No: 70, and a the amino acid sequence of CDR3 comprises the amino acid sequence set forth as SEQ ID No: 71;
l) a the amino acid sequence of CDR1 comprises the amino acid sequence set forth as SEQ ID No: 72, a the amino acid sequence of CDR2 comprises the amino acid sequence set forth as SEQ ID No: 73, and a the amino acid sequence of CDR3 comprises the amino acid sequence set forth as SEQ ID No: 74;
m) the amino acid sequence of CDR1 comprises the amino acid sequence set forth as SEQ ID No: 75, a the amino acid sequence of CDR2 comprises the amino acid sequence set forth as SEQ ID No: 76, and a the amino acid sequence of CDR3 comprises the amino acid sequence set forth as SEQ ID No: 77; or
n) the amino acid sequence of CDR1 comprises the amino acid sequence set forth as SEQ ID No: 78, the amino acid sequence of CDR2 comprises the amino acid sequence set forth as SEQ ID No: 79, and a the amino acid sequence of CDR3 comprises the amino acid sequence set forth as SEQ ID No: 80.

22. (canceled)

23. A method of treating a disease in a subject, comprising administering to the subject an effective amount of a bivalent molecule of claim 1.

24. (canceled)

25. The method of claim 23, wherein the disease is an inherited ion channelopathy, a cancer, a cardiovascular condition, an infectious disease, or a metabolic disease.

26. (canceled)

27. (canceled)

28. A method of identifying and preparing a nanobody binder targeting a protein of interest, comprising:
a) constructing a naive yeast library that expresses synthetic nanobodies;
b) incubating the naive yeast library with the protein of interest;
c) selecting yeast cells expressing nanobodies that bind to the protein of interest by magnetic-activated cell sorting (MACS);
d) amplifying the selected cells and constructing an enriched yeast library;
e) incubating the enriched yeast library with the protein of interest;
f) selecting yeast cells expressing nanobodies that bind to the protein of interest by fluorescence activated cell sorting (FACS);
g) amplifying the selected cells and constructing a further enriched yeast library;
h) repeating steps e) to g) twice; and
i) sorting the selected yeast cells as single cells and cultivating as monoclonal colonies for binding validation and plasmid isolation.

29. (canceled)

30. (canceled)

31. The bivalent molecule of claim 1, wherein the DUB is an OTU family member.

32. The bivalent molecule of claim 5, wherein the target binder is a nanobody.

33. A polynucleotide encoding the bivalent molecule of claim 1.

34. A vector comprising the polynucleotide of claim 31.

35. A host cell comprising the vector of claim 32.

36. A method of treating a disease in a subject, comprising administering to the subject an effective amount of the polynucleotide of claim 33.

37. A method of treating a disease in a subject, comprising administering to the subject an effective amount of the vector of claim 34.
